(19) United States
(12) Patent Application Publication
(10) Pub. No.: US 2016/0151639 A1
(43) Pub. Date: Jun. 2, 2016

(54) DEVICES AND METHODS FOR DELIVERY OF THERAPEUTIC ENERGY

(71) Applicant: Oxys AG, Horgen (CH)

(72) Inventors: Christoph Scharf, Horgen (CH); Gunter Scharf, Gockhausen (CH); J. Christopher Flaherty, Auburndale, FL (US)

(73) Assignee: Oxys AG, Horgen (CH)

(21) Appl. No.: 14/898,599

(22) PCT Filed: Jul. 8, 2014
(86) PCT No.: PCT/US14/45722

§ 371 Date: Dec. 15, 2015

(30) Foreign Application Priority Data
Jul. 10, 2013 (CH) 1236/13
Nov. 19, 2013 (CH) 1928/13

(51) Int. Cl. A61N 5/06; A61B 18/18

(52) U.S. Cl. CPC A61N 5/0601; A61N 5/0624; A61N 5/0625; A61B 18/18; A61B 2018/00589

(57) ABSTRACT

A device for insertion into a mammalian patient comprises a shaft and a light delivery element. The shaft comprises a proximal end, a distal end, and a lumen therebetween. The light delivery element is constructed and arranged to deliver light to prevent infection, reduce infection and/or cause a therapeutic benefit or physiologic effect.

### RELATED APPLICATIONS

This application claims priority from Swiss Provisional Application Serial Number 1236/13, filed Jul. 10, 2013, and Swiss Provisional Application Serial Number 1928/13, filed Nov. 19, 2013, the entire disclosures of which are incorporated herein be reference.

### FIELD OF THE INVENTION

The present invention relates generally to devices that deliver energy to reduce infection risk or provide a therapeutic benefit to a patient.

### BACKGROUND

Medical devices, such as those introduced into an internal body location, can be associated with a risk of infection. For example, the vast majority (over 95%) of hospital acquired urinary tract infections are catheter associated. The majority (over 80%) of blood stream infections are associated with intravenous catheters. Any foreign body, either temporarily or permanently inserted into the body, carries the risk of bacterial colonization and subsequent infection. The development of a biofilm containing different species of bacteria, yeasts and other pathogens is an ideal niche for exchange for bacterial resistance genes among species, and thus the biofilm is an ideal niche for exchange of antibiotic resistance among bacteria. The emergence of bacteria resistant to multiple pharmaceutical agents is a growing health care problem and some hospital bacteria have become resistant to almost every available antibiotic treatment. Care and treatment of the infection is expensive and burdensome. There is a need for devices and methods to reduce or prevent device related infections.

There is also a need for devices and methods that deliver energy to blood, to treat one or more patient diseases or disorders.

### SUMMARY

According to one aspect of the present inventive concepts, a device for insertion into a mammalian patient comprises a shaft and a light delivery element. The shaft comprises a proximal end and a distal end. One or more lumens may extend between the proximal end and the distal end. The shaft further comprises an outer surface and an inner surface. The light delivery element is constructed and arranged to deliver light to perform a function selected from the group consisting of: prevent infection; reduce infection; cause a therapeutic benefit or other physiologic effect; and combinations thereof. The physiologic effect can be selected from the group consisting of: blood temperature increase; vasodilation; increase in local nitric oxide; enhance nitric oxide release from the vascular endothelium; prolong local nitric oxide effects; alteration in the function of erythrocytes; modification in oxygen release from hemoglobin; modification in pH of blood; modulation in the immune response of blood leucocytes; modulation of the coagulation and/or thrombocyte function; modification in the function of heme catalyst enzymes in the blood; modification in hormonal action of a peptide and/or non-peptide hormone; modification in the binding capacity of one or more antibodies; decrease in blood glucose level; affect circulating tumor cells by selective heating, photocoagulation and/or photolysis; and combinations thereof. The device can be constructed and arranged for insertion into the bladder of the patient, such as to deliver light to one or more of the bladder, the urethra, and/or urine in the bladder and/or urethra.

In some embodiments, the device is constructed and arranged to prevent urinary tract infections.

In some embodiments, the device is constructed and arranged to be inserted into the urethra of the patient.

In some embodiments, the device is constructed and arranged to be inserted into the bladder of the patient, such as to evacuate urine from the patient.

In some embodiments, the device is constructed and arranged to be inserted into a blood vessel of the patient, such as to at least one of withdraw blood from a blood vessel or deliver material to a blood vessel.

In some embodiments, the device is constructed and arranged to be at least one of inserted into the heart of the patient or placed proximate the heart of the patient. The device can be constructed and arranged to provide at least one of pacing or defibrillating energy to the heart.

In some embodiments, the device comprises an implanted agent delivery pump. The pump can comprise a catheter constructed and arranged to deliver an agent, and the light delivery element can be positioned at least one of on or in the catheter. The agent delivered can be an agent selected from the group consisting of: neurological agent; pain control agent such as morphine; a chemotherapeutic; insulin; and combinations thereof.

In some embodiments, the device comprises an external agent delivery pump. The pump can comprise a transcutaneous conduit configured to deliver the agent. The delivered agent can comprise an agent selected from the group consisting of: insulin; a chemotherapeutic agent; a nutritional material; and combinations thereof. The light delivery element can be positioned at least one of on or in the transcutaneous conduit.

In some embodiments, the device comprises a device selected from the group consisting of: urine removal catheter; bladder catheter; urethral catheter; nephrostomy catheter; vascular access device; central venous catheter; peripherally inserted central catheter; insulin pump; implanted device; implanted drug delivery pump; pacemaker; neurostimulator; artificial heart; drainage catheter; colostomy tube; and combinations thereof.

In some embodiments, the device is constructed and arranged for short-term clinical use, such as use for less than 16 hours, less than 24 hours, less than 3 days or less than 7 days. In other embodiments, the device is constructed and arranged for long-term clinical use, such as use for at least 1 week, at least 1 month, at least 3 months, or at least 6 months.

In some embodiments, the light delivery element is further constructed and arranged to produce the light to be delivered. The light delivery element can comprise a light producing element selected from the group consisting of: an LED; a lamp; a laser; and combinations thereof.

In some embodiments, the light delivery element is constructed and arranged to direct light toward at least one of tissue or a bodily fluid. The tissue can comprise blood. The light delivery element can be constructed and arranged to deliver light toward a material selected from the group consisting of: blood; bladder wall tissue; urethral wall tissue; urine; esophageal tissue; airway tissue; subcutaneous tissue; vascular wall tissue; cardiac valve tissue; cerebrospinal fluid; meningeal tissue; synovial fluid; and combinations thereof. The light delivery element can be constructed and arranged to direct light toward an incision in the patient's skin.

In some embodiments, the light delivery element is constructed and arranged to direct light toward a body fluid. The body fluid can comprise urine.

In some embodiments, the light delivery element is constructed and arranged to deliver light to at least one of prevent, eliminate or reduce colonization of foreign material by at least one of: bacteria; virus; fungi or a parasite. The light delivery element can be constructed and arranged to deliver light to at least one of prevent, eliminate or reduce a biofilm of bacteria. The light delivery element can be constructed and arranged to deliver light at a power between approximately 1.0 mW and 100 mW. The light delivery element can be constructed and arranged to deliver light to tissue at a power density of between approximately 1.0 mW/cm² and 10.0 mW/cm².

In some embodiments, the light delivery element is constructed and arranged to deliver light to at least one of reduce or eliminate a bacteria selected from the group consisting of: Escherichia coli; Klebsiella; Pseudomonas and other gram negative intestinal bacteria; Staphylococcus aureus; Streptococcus; a skin bacteria; Pneumococcus; Hämophilus; a respiratory tract bacteria; anaerobic bacteria; and combinations thereof. In some embodiments, the light delivery element is constructed and arranged to deliver light to at least one of reduce or prevent an infection resulting from one or more of: a virus; a fungus; or a parasite.

In some embodiments, the light delivery element is constructed and arranged to deliver light to the bladder of the patient. The light delivery element can be constructed and arranged to deliver light to the majority of the cavity of the bladder. The light delivery element can be constructed and arranged to deliver light to urine within the bladder. The light delivery element can be further constructed and arranged to further deliver light to the urethra. The light delivery element can be further constructed and arranged to deliver light to urine in the urethra.

In some embodiments, the light delivery element is constructed and arranged to deliver light radially outward from the at least a portion of the outer surface. The light delivery element can be constructed and arranged to deliver light radially outward from a majority of the outer surface. The light delivery element can be constructed and arranged to further deliver light radially inward from the at least a portion of the inner surface. The light delivery element can be constructed and arranged to deliver light to an external surface portion of the patient's skin. The external surface portion can comprise a portion of skin surrounding the urethral orifice. The external surface portion can comprise a portion of skin surrounding a skin penetration site.

In some embodiments, the light delivery element is constructed and arranged to deliver light radially inward from the at least a portion of the inner surface. The light delivery element can be constructed and arranged to deliver light radially inward from the at least a portion of the inner surface. The light delivery element can be constructed and arranged to deliver light to an external surface portion of the patient's skin. The external surface portion can comprise a portion of skin surrounding the urethral orifice. The external surface portion can comprise a portion of skin surrounding a skin penetration site.

In some embodiments, the light delivery element is constructed and arranged to be reused. The device can further comprise a second shaft, and the light delivery element can be constructed and arranged to be used with the first shaft in a first use and the second shaft in a second use.

In some embodiments, the light delivery element is constructed and arranged to deliver light to the patient for short-term clinical use, such as use for less than 16 hours, less than 24 hours, less than 3 days, or less than 7 days. The light delivery element can be constructed and arranged to deliver the light at least one of continuously or intermittently.

In some embodiments, the light delivery element is constructed and arranged to deliver light to the patient for long-term clinical use, such as use for at least 7 days, at least 1 month, at least 3 months, or at least 6 months. The light delivery element can be constructed and arranged to deliver the light at least one of continuously or intermittently. The light delivery element can be constructed and arranged to be implanted in the patient.

In some embodiments, the light delivery element is constructed and arranged to be rotated. The device can further comprise a rotating assembly constructed and arranged to rotate the light delivery element. The rotating assembly can be constructed and arranged to rotate the light delivery element at least 360°.

In some embodiments, the light delivery element comprises light scattering material. The light scattering material can comprise a material selected from the group consisting of: alumina particles; silica particles; titania particles; titanium oxide particles; and combinations thereof. The light delivery element can further comprise a polymeric material wherein the light scattering material is embedded throughout the polymeric material. The polymeric material can comprise silicone.

In some embodiments, the light delivery element comprises an optical element. The optical element can be constructed and arranged to couple light into the light delivery element. The optical element can be constructed and arranged to distribute light from the light delivery element. The light delivery element can comprise an optical element selected from the group consisting of: optical fiber; lens; ball lens; prism; diffractor; filter; mirror; and combinations thereof. The device can further comprise at least one optical fiber with a proximal end, at least one cladded segment and at least one uncladded segment. The proximal end can be constructed and arranged to be optically connected to a light source and the light delivery element can comprise the at least one uncladded segment. The at least one optical fiber can comprise an array of optical fibers positioned within the shaft. The device can comprise at least one optical fiber, and the optical element can be positioned at the end of the optical fiber, such as when the optical element comprises a ball lens positioned at the end of the fiber. The optical element can be constructed and arranged to be rotated. The device can further comprise a rotating assembly constructed and arranged to rotate the optical element.

In some embodiments, the light delivery element comprises an optical element constructed and arranged to direct light toward a majority of the surface of the bladder. The optical element can comprise an element selected from the group consisting of: lens; prism; diffractor; mirror; and combinations thereof.

In some embodiments, the light delivery element comprises a fluorescent material. The fluorescent material can be constructed and arranged to increase light dispersion. The fluorescent material can be positioned on at least one a portion of the shaft outer surface or a portion of the shaft inner surface. The fluorescent material can comprise a coating.

In some embodiments, the light delivery element comprises a coating. The coating can comprise a photosensitizer. The coating can comprise a material constructed and arranged to be activated by light to provide a bactericidal effect.

In some embodiments, the shaft is constructed and arranged to pass through an incision on the surface of the skin. In these embodiments, the device can be constructed and arranged to further pass into a blood vessel. Alternatively or additionally, the device can be constructed and arranged to further pass into a subcutaneous tissue tunnel. Alternatively or additionally, the device can be constructed and arranged to further pass into an organ, such as the kidney. Alternatively or additionally, the device can be constructed and arranged to further pass into the ureter. Alternatively or additionally, the device can be constructed and arranged to further pass into at least one of: a blood vessel; a ventricle of the brain; a portion of the cerebrospinal fluid space; a joint capsule; or a chamber of the heart.

In some embodiments, the shaft is constructed and arranged to pass through a body opening. The body opening can comprise an opening selected from the group consisting of: urethra; mouth; anus; vagina; nostril; ear hole; eye socket; and combinations thereof. The shaft can be constructed and arranged for insertion into the bladder of the patient. The shaft can be constructed and arranged to remove urine from the patient's bladder.

In some embodiments, the shaft is constructed and arranged to be inserted into at least one of: a blood vessel; a ventricle of the brain; a portion of the cerebrospinal fluid space; a joint capsule; a chamber of the heart; or an organ such as the bladder or kidney.

In some embodiments, at least a portion of the shaft comprises a translucent material. The translucent material can surround at least a portion of the light delivery element. The light delivery element can comprise a portion of an optical fiber that is not surrounded by cladding material.

In some embodiments, the shaft comprises a flexible shaft. Alternatively, the shaft can comprise a rigid shaft. In some embodiments, the shaft comprises at least a first portion that is rigid and a second portion that is flexible.

In some embodiments, the shaft comprises a diameter between approximately 3 mm and 9 mm.

In some embodiments, the lumen is constructed and arranged to provide a function selected from the group consisting of: withdrawing blood; delivering agent to an internal body location of the patient; and combinations thereof. The lumen can be constructed and arranged to deliver an agent constructed and arranged to improve the therapeutic effect of light delivered by the light delivery element.

In some embodiments, the device further comprises at least one conductor positioned within the lumen. The at least one conductor can be constructed and arranged to deliver energy to the heart.

In some embodiments, the lumen comprises a first lumen and wherein the shaft further comprises at least a second lumen. The first lumen and the second lumen can be constructed and arranged to deliver different agents into the cardiovascular system of the patient.

In some embodiments, the device further comprises at least one optical fiber constructed and arranged to deliver light to the light delivery element. The light delivery element can comprise the at least one optical fiber. The light delivery element can comprise at least a portion of the at least one optical fiber. The at least one optical fiber can comprise a first portion surrounded by cladding and a second portion not surrounded by cladding, and the light delivery element can comprise the second portion not surrounded by cladding. The at least a portion of the at least one optical fiber can comprise a surface modified to enhance distribution. The modified surface can comprise a roughened surface. The modified surface can comprise a surface receiving a surface treatment selected from the group consisting of: etching; cutting; covering with roughened material such as silicon; and combinations thereof. The at least one optical fiber can comprise a material selected from the group consisting of: glass; plastic; polymethylmethacrylate (PMMA); one or more polymers such as one or more polymers configured as a microstructured polymer optical fiber; photonic crystal; polycarbonate; polystyrene; and combinations thereof. The at least one optical fiber can comprise a flexible optical fiber. The at least one optical fiber can comprise multiple optical fibers. The multiple optical fibers can comprise a first optical fiber constructed and arranged to deliver light to the bladder and a second optical fiber constructed and arranged to deliver light to the urethra. The device can be constructed and arranged to deliver more light to the bladder than to the urethra. The at least one optical fiber can be positioned within the shaft. The at least one optical fiber can be positioned in the lumen of the shaft. The shaft can comprise at least a second lumen positioned between the outer surface and the inner surface, and the at least one optical fiber can be insertable into the second lumen. Alternatively or additionally, the at least one optical fiber can be positioned (e.g. fixed) in the second lumen. The device can further comprise cladding surrounding at least a portion of the at least one optical fiber. The at least one fiber can comprise a distal portion that extends beyond the cladding. The light delivery element can comprise the at least one fiber distal portion. The at least one fiber distal portion can extend at least 2 cm beyond the cladding, or at least 6 cm beyond the cladding. The light delivery element can comprise a first optical component connected to a first optical fiber and a second optical component connected to a second optical fiber. The first optical component can comprise light scattering material. The light scattering material can be constructed and arranged to direct light radially out from the shaft outer surface. The light scattering material can be constructed and arranged to direct light radially in from the shaft inner surface. The second optical component can comprise an optical element configured to direct light toward the majority of the inner surface of the bladder. The shaft can comprise at least a translucent portion and wherein the at least one fiber is constructed and arranged to deliver light to the at least a translucent portion.

In some embodiments, the device further comprises a light source. The light source can be constructed and arranged to produce light to at least one of reduce or eliminate a bacteria selected from the group consisting of: Escherichia coli; Klebsiella; Pseudomonas and other gram negative intestinal bacteria; Staphylococcus aureus; Streptococcus; a skin bacteria; Pneumococcus; Hämophilus; a respiratory tract bacteria; anaerobic bacteria and combinations thereof. The light source can be constructed and arranged to produce or otherwise provide light to at least one of reduce or prevent an infection resulting from one or more of: a virus; a fungus; or a parasite. The light source can comprise at least one LED, such as at least one organic LED. Alternatively or additionally, the light source can comprise a laser. The light source can be constructed and arranged to deliver pulsed light. The light source can be constructed and arranged to deliver light at a duty cycle between approximately 0.1% and 50%. The pulsed light can comprise a first pulse of light at a first wavelength and a second pulse of light at a second wavelength. The light source can be constructed and arranged to deliver light at a power less than or equal to 100 Watts, such as at a power less than or equal to 50 Watts. The light source can be constructed and arranged to deliver pulse-width modulated light to the light delivery element. The light source can be constructed and arranged to deliver light at a power less than or equal to 50 Watts rms, such as at a power less than or equal to 20 Watts rms. The light source can be constructed and arranged to deliver light at a power of more than or equal to 0.1 Watts. The light source can be constructed and arranged to deliver light at a power between 2 Watts and 10 Watts. The light source can be constructed and arranged to deliver light at a power between 1 mW and 100 mW. The light source can be constructed and arranged to deliver light at a wavelength between approximately 300 nanometers and 900 nanometers, such as between approximately 400 nanometers and 750 nanometers or between approximately 400 nanometers and 430 nanometers. The light source can be constructed and arranged to deliver visible light. The light source can be constructed and arranged to deliver ultraviolet light. The light source can be constructed and arranged to deliver light at varying wavelengths. The delivered light can comprise continuously varying wavelengths. The delivered light can comprise light alternating between at least a first wavelength and a second wavelength. The light source can be constructed and arranged to deliver light at multiple wavelengths simultaneously. The device can be constructed and arranged to deliver light to tissue at a power density less than or equal 500 milliwatts/cm² (mW/cm2), such as at a power density less than or equal 250 mW/cm², or less than or equal 100 mW/cm². The device can be constructed and arranged to deliver light to tissue at a power density of approximately 100 mW/cm². In some embodiments, the device is constructed and arranged to deliver light to tissue at a power density of between 1.0 mW/cm² and 10 mW/cm². The device can be constructed and arranged to deliver light to tissue at a level constructed and arranged to prevent mucosal dehydration or other tissue. The light source can be constructed and arranged to deliver pulse-width modulated light. The light source can be operably connected to the light delivery element. The device can further comprise at least one optical fiber, and the light source can be operably connected to the light delivery element by the at least one optical fiber. The device can further comprise a handle on the proximal end of the shaft. The light source and the handle can be constructed and arranged to be operably attached by a user. Alternatively or additionally, the light source can be positioned in the handle. The light source can be positioned within the shaft. The device can comprise a balloon. The light source can be positioned within the balloon, such as when the light source comprises at least one LED positioned within the balloon. The light source can comprise a battery. The battery can comprise a replaceable and/or rechargeable battery. The light source can be constructed and arranged to be reused.

In some embodiments, the device further comprising a light enhancing material selected from the group consisting of: a photosensitizer; a photocatalyst; and combinations thereof. The light enhancing material can comprise a material selected from the group consisting of: toluidine blue O; methylene blue; and combinations thereof. The device can be constructed and arranged to deliver the light enhancing material. The light enhancing material can be constructed and arranged to be activated by light delivered by the device. The light enhancing material can be constructed and arranged to be activated by light delivered by the light delivery element.

In some embodiments, the device further comprises an anchoring element constructed and arranged to anchor at least a portion of the shaft in an internal body location. The anchor can be constructed and arranged to anchor the at least a portion of the shaft in the bladder. The anchor can comprise a balloon.

In some embodiments, the device further comprises a sensor, or multiple sensors. The sensor can be constructed and arranged to regulate the light delivered. The sensor can be constructed and arranged to at least one of prevent or reduce tissue damage. The sensor can be constructed and arranged to at least one of prevent or reduce mucosal dehydration. The sensor can comprise a temperature sensor. The temperature sensor can comprise at least one of a thermocouple or a thermistor. The temperature sensor can comprise at least one optical fiber constructed and arranged to gather infrared light. The sensor can be positioned at least one of on or within the shaft. The device can further comprise a balloon mounted to the shaft, and the sensor can be positioned at least one of on or within the balloon. The device can further comprise a temperature measurement assembly constructed and arranged to determine a measured temperature based upon one or more signals produced by the sensor. The temperature measurement assembly can be constructed and arranged to be operably attachable to the sensor.

In some embodiments, the device further comprises a functional element constructed and arranged to at least one of further prevent or further reduce an infection. The functional element can comprise an electromagnetic field delivery element, and the electromagnetic field generated can be constructed and arranged to at least one of further prevent or further reduce an infection. The electromagnetic field can comprise at least one of: a dynamic electromagnetic field; a static electromagnetic field; a dynamic magnetic field; or a static magnetic field. The electromagnetic field delivery element can comprise at least one permanent magnet, such as multiple permanent magnets dispersed relatively uniformly along the length of the shaft. The electromagnetic field can comprise a magnetic field with a field strength between 1 mT and 500 mT. The electromagnetic field can comprise a magnetic field constructed and arranged to prevent adversely effecting at least one of a muscle or a nerve. Alternatively or additionally, the functional element can comprise an electric current and/or electric potential delivery element, and the electric current and/or electric potential delivered can be constructed and arranged to at least one of further prevent or further reduce an infection. The electric current and/or electric potential delivered can be constructed and arranged to cause electrolysis. Alternatively or additionally, the functional element can comprise an ultrasound transducer, and the ultrasound waves produced can be constructed and arranged to at least one of further prevent or further reduce an infection. The device can further comprise a source of power, and the functional element can be operatively attached to the source of power. At least two wires can connect the functional element to the power source, such as when the functional element is constructed and arranged to provide an electromagnetic field and/or an electrical field (e.g. to tissue and/or body fluid). The device can comprise an expandable element such as a balloon, and the functional element can be positioned at least one of on or in the expandable element. The expandable element can be constructed and arranged for positioning and/or anchoring in the bladder. The functional element can comprise a first functional element and a second functional element. The first functional element and the second functional element can singly, or in combination, be constructed and arranged to at least one of further prevent or further reduce infection.

In some embodiments, the at least one energy delivery element is constructed and arranged to deliver energy at a wavelength based on one or more microorganisms to be treated.

In some embodiments, the wavelength comprises approximately 405 nm and the microorganism to be treated comprises E. Coli.

In some embodiments, the wavelength comprises approximately 470 nm and the microorganism to be treated comprises staphylococcus.

In some embodiments, the wavelength comprises approximately 670 nm and the microorganism to be treated comprises oral candida.

In some embodiments, the light delivery element comprises one or more light delivery elements each optically attached to one or more optical fibers.

In some embodiments, the one or more fibers terminate in a wall of the shaft.

In some embodiments, the one or more fibers comprise a first refraction index and the shaft comprises a second refraction index similar to the first refraction index.

In some embodiments, the shaft comprises light scattering material such as titanium dioxide.

In some embodiments, the device further comprises a balloon, wherein the light delivery elements deliver light to the balloon.

In some embodiments, the device further comprises light scattering material such as titanium dioxide positionable within the balloon.

In some embodiments, the light scatting material is mixed with a fluid such as water and/or saline.

In some embodiments, the balloon comprises an inner surface, an outer surface and a wall. The device further comprises light scattering material such as titanium dioxide positioned on the inner surface, the outer surface and/or within the wall of the balloon.

In some embodiments, the device further comprises light scattering material constructed and arranged to enhance hydrolysis.

In some embodiments, the hydrolysis is constructed and arranged to generate oxygen radials that enhance the bactericidal effects of light delivered by the device.

In some embodiments, the device further comprises a photocatalyst.

In some embodiments, the photocatalyst comprises vanadium pentoxide.

In some embodiments, the device is constructed and arranged to deliver light to one or more patient locations prior to any significant bacterial presence.

According to another aspect of the present inventive concepts, a method of preventing or reducing infection comprises selecting a device of the present inventive concepts. The method further comprises performing at least one of: attaching the device to the patient; inserting the device into a natural orifice of the patient; inserting the device through a skin incision in the patient; or implanting at least a portion of the device in the patient.

In some embodiments, the method further comprises directing light from the light delivery element toward at least one of tissue or body fluid. The at least one of tissue or body fluid can comprise a material selected from the group consisting of: blood; bladder wall tissue; urethral wall tissue; urine; esophageal tissue; airway tissue; subcutaneous tissue; vascular wall tissue; cardiac valve tissue; cerebrospinal fluid; meningeal tissue; synovial fluid; and combinations thereof.

In some embodiments, the method further comprises inserting at least a portion of the shaft into the urethra and into the bladder. The method can further comprise removing urine from the patient.

In some embodiments, the method further comprises implanting at least a portion of the device in the patient. The method can further comprise delivering energy to an internal patient location. The delivered energy can comprise at least one of pacing or defibrillating energy delivered to the heart.

In some embodiments, the method further comprises inserting at least a portion of the device into the patient's cardiovascular system. The method can further comprise at least one of delivering an agent to, or withdrawing blood, from the patient's cardiovascular system.

In some embodiments, the method further comprises at least one of further preventing or further reducing infection by delivering one or more of: an electromagnetic field; an electric current and/or electric potential; or ultrasound waves.

According to another aspect of the present inventive concepts, a device for insertion into a mammalian patient comprises a shaft and a functional element. The shaft comprises a proximal end, a distal end and a lumen therebetween. The shaft further comprises an outer surface and an inner surface. The functional element is constructed and arranged at least one of prevent or reduce infection. The device can be constructed and arranged for insertion into the bladder of the patient, such as to deliver light to one or more of the bladder, the urethra, and/or urine in the bladder and/or urethra.

In some embodiments, the device further comprises one or more light delivery elements constructed and arranged to at least one of further prevent or further reduce an infection.

In some embodiments, the functional element comprises an electromagnetic field delivery element, and the electromagnetic field generated can be constructed and arranged to at least one of further prevent or further reduce an infection. The electromagnetic field can comprise at least one of: a dynamic electromagnetic field; a static electromagnetic field; a dynamic magnetic field; or a static magnetic field. The electromagnetic field delivery element can comprise at least one permanent magnet. The electromagnetic field delivery element can comprise multiple permanent magnets, such as multiple permanent magnets dispersed relatively uniformly along the length of the shaft. The electromagnetic field can comprise a magnetic field with a field strength between 1 mT and 500 mT. The electromagnetic field can be constructed and arranged to prevent adversely effecting at least one of a muscle or a nerve.

In some embodiments, the functional element comprises an electric current and/or electric potential delivery element, and the electric current and/or electric potential delivered can be constructed and arranged to at least one of further prevent or further reduce an infection. The electric current and/or electric potential delivered can be constructed and arranged to cause electrolysis.

In some embodiments, the functional element comprises an ultrasound transducer, and the ultrasound waves produced by the transducer can be constructed and arranged to at least one of further prevent or further reduce an infection.

In some embodiments, the device further comprises a source of power, and the functional element can be operatively attached to the source of power. The device can further comprise at least two wires connecting the functional element to the power source. The functional element can be constructed and arranged to provide an electromagnetic field and/or an electric field.

In some embodiments, the device further comprises an expandable element, and the functional element can be positioned at least one of on or in the expandable element. The expandable element can comprise a balloon, such as a balloon constructed and arranged to be positioned in the bladder.

In some embodiments, the functional element comprises a first functional element and a second functional element, and the first functional element and the second functional element can be each constructed and arranged to at least one of further prevent or further reduce infection.

According to another aspect of the present invention, a device for insertion into a mammalian patient comprises a shaft comprising a proximal end and a distal portion, and at least one energy delivery element. The at least one energy delivery element can be constructed and arranged to cause a physiologic effect selected from the group consisting of: blood temperature increase; vasodilation; increase in local nitric oxide; enhance in nitric oxide release from the vascular endothelium; prolong local nitric oxide effects; alteration in the function of erythrocytes; modification in oxygen release from hemoglobin; modification in pH of blood; modulation in the immune response of blood leucocytes; modulation of the coagulation and/or thrombocyte function; modification in the function of heme catalyst enzymes in the blood; modification in hormonal action of a peptide and/or non-peptide hormone; modification in the binding capacity of one or more antibodies; decrease in blood glucose level; affect circulating tumor cells by selective heating, photocoagulation and/or photolysis; and combinations thereof.

In some embodiments, the shaft is constructed and arranged for insertion into at least one blood vessel of the patient.

In some embodiments, the device is constructed and arranged to cause electrolysis of blood to prevent clotting proximate the device.

In some embodiments, the at least one energy delivery element is constructed and arranged to cause vasodilation.

In some embodiments, the at least one energy delivery element is constructed and arranged to cause an increase in local nitric oxide.

In some embodiments, the at least one energy delivery element is constructed and arranged to cause an alternation in the function of erythrocytes.

In some embodiments, the at least one energy delivery element is constructed and arranged to cause a modification in oxygen release from hemoglobin. The modification can be caused by interfering with a heme-heme interaction. The modification can be caused by the electrostatic stabilization of deoxyhemoglobin in the T-form. The modification can be caused by increasing the blood temperature.

In some embodiments, the at least one energy delivery element is constructed and arranged to cause a modification in pH of blood. The modification in pH can comprise and increase in pH. The modification in pH can comprise and decrease in pH.

In some embodiments, the at least one energy delivery element is constructed and arranged to cause a modulation in the immune response of blood leucocytes. The immune response of blood leucocytes can be constructed and arranged to prevent at least one of: organ rejection; graft versus host disease; or autoimmune disease.

In some embodiments, the at least one energy delivery element is constructed and arranged to cause a modulation of the coagulation and/or thrombocyte function. The coagulation and/or thrombocyte function can be constructed and arranged to increase coagulability. The coagulation and/or thrombocyte function can be constructed and arranged to inhibit coagulability.

In some embodiments, the at least one energy delivery element is constructed and arranged to cause a modification in the function of heme catalyst enzymes in the blood. The functionally modified enzyme can comprise an enzyme selected from the group consisting of: catalase; endothelial nitric oxide synthase (ENOS); cytochrome; myoglobin; and combinations thereof.

In some embodiments, the at least one energy delivery element is constructed and arranged to cause a modification in hormonal action of a peptide and/or non-peptide hormone.

In some embodiments, the at least one energy delivery element is constructed and arranged to cause a modification in the binding capacity of one or more antibodies.

In some embodiments, the at least one energy delivery element is constructed and arranged to cause a decrease in blood glucose level. The decrease in blood glucose level can be caused by an electrochemical reaction. The device can further comprise a reagent, and the device can be constructed and arranged to deliver the reagent and the reagent can be constructed and arranged to cause the blood glucose level to decrease.

In some embodiments, the at least one energy delivery element is constructed and arranged to deliver energy selected from the group consisting of: electromagnetic energy; light energy; electrostatic field energy; magnetic field energy; varying field energy; microwave energy; ultrasound energy; and combinations thereof.

In some embodiments, the at least one energy delivery element is constructed and arranged to deliver light to at least one of blood in a blood vessel; blood in a heart chamber; or a blood vessel wall.

In some embodiments, the at least one energy delivery element comprises a light delivery element. The at least one energy delivery element can further comprise a field delivery element constructed and arranged to deliver energy selected from the group consisting of: an electrostatic field; a magnetic field; an electromagnetic field; and combinations thereof.

In some embodiments, the at least one energy delivery element comprises a field delivery element constructed and arranged to deliver energy selected from the group consisting of: an electrostatic field; a magnetic field; an electromagnetic field; and combinations thereof.

In some embodiments, the at least one energy delivery element comprises at least one microwave energy delivery element.

In some embodiments, the at least one energy delivery element comprises at least one ultrasound energy delivery element.

In some embodiments, the device is constructed and arranged to deliver pulsed energy.

In some embodiments, the device is constructed and arranged to adjust energy delivered by the at least one energy delivery. The energy adjustment can comprise an adjustment to the field strength of the electromagnetic energy delivery. The energy adjustment can comprise an adjustment to a pulse-width modulation parameter of the energy delivery. The device can further comprise a sensor constructed and arranged to produce a signal, the device can be constructed and arranged to perform the energy adjustment based on the sensor signal. The sensor can comprise a sensor selected from the group consisting of: temperature sensor such as a thermocouple or thermister; oxygen sensor; glucose sensor such as an optical glucose sensor; pH sensor; physiologic sensor; pressure sensor; blood gas sensor; blood conductivity sensor; impedance sensor; motion sensor; accelerometer; and combinations thereof. The device can be constructed and arranged to perform the energy adjustment based on at least one of the conductivity or the impedance of blood.

In some embodiments, the at least one energy delivery element is positioned on the shaft distal portion.

In some embodiments, the shaft further comprises a distal end and the at least one energy delivery element is positioned proximate the shaft distal end.

In some embodiments, the at least one energy delivery element is advanceable from the shaft. The shaft can further comprise a distal end and the at least one energy delivery element is advanceable from the shaft distal end. The shaft can further comprise an outer wall the at least one energy delivery element can be advanceable from the shaft outer wall.

In some embodiments, the at least one energy delivery element comprises multiple energy delivery elements. The shaft can comprise a first shaft and the device can further comprise a second shaft, the at least one energy delivery element can comprise a first energy delivery element positioned on the first shaft and a second energy delivery element positioned on the second shaft. The device can further comprise a third shaft, and the at least one energy delivery element can further comprise a third energy delivery element positioned on the third shaft.

In some embodiments, the device further comprises an agent. The device can be constructed and arranged to deliver the agent. The agent can comprise a material selected from the group consisting of: photocatalyst; photosensitizer; and combinations thereof. The at least one energy delivery element can be constructed and arranged to produce a photodynamic effect and the agent can be constructed and arranged to enhance the photodynamic effect. The agent can comprise a material selected from the group consisting of: metal; protein; carbohydrate; fatty acid; a nucleic acid; a synthetic medication; antibody; ionic solution; heme group; flavin group; aminolaevulin; phenol; a polycarbon; and combinations thereof.

In some embodiments, the device further comprises a sensor constructed and arranged to produce a signal. The device can be constructed and arranged to adjust the energy delivered by the at least one energy delivery element based on the sensor signal. The sensor can comprise a sensor selected from the group consisting of: temperature sensor such as a thermocouple or thermister; oxygen sensor; glucose sensor such as an optical glucose sensor; pH sensor; physiologic sensor; pressure sensor; blood gas sensor; blood conductivity sensor; and combinations thereof. The device can be constructed and arranged to adjust the delivery of light energy based on the sensor signal. The device can be constructed and arranged to adjust the delivery of electromagnetic field energy based on the sensor signal. The sensor can be constructed and arranged to measure blood glucose. The sensor can comprise an optical sensor. The sensor can comprise oxygen sensor.

In some embodiments, the device further comprises a balloon, an inflation lumen fluidly attached to the balloon, and an inflation port fluidly attached to the inflation lumen. The device can further comprise a valve constructed and arranged to maintain fluid in the balloon. The valve can be positioned in the inflation port.

In some embodiments, the device further comprises light scattering material and a balloon with an outer surface and an inner surface. The light scattering material can be positioned at a location selected from: on the outer surface of the balloon; on the inner surface of the balloon; within the balloon; and combinations thereof.

In some embodiments, the device further comprises a balloon and injectable material for positioning within the balloon. The injectable material can comprise a material selected from the group consisting of: a light scattering material; a material configured to produce a photomagnetic effect in the presence of light; and combinations thereof. The injectable material can comprise both a light scattering material and a material configured to produce a photomagnetic effect in the presence of light. The injectable material can be configured to produce a photomagnetic effect that at least reduces adhesion of bacteria. The injectable material can comprise an analogue of Prussian Blue.

In some embodiments, the device further comprises a power supply electrically attached to a light delivery element, wherein the power supply is flexibly attached to the shaft such as to avoid imparting significant force to the shaft and/or the patient. In some embodiments, the device further comprises an indicator configured to alert an operator that the light delivery element is delivering light. In some embodiments, the device further comprises a cooling element constructed and arranged to dissipate heat form the light delivery element. The cooling element can comprise an element selected from the group consisting of: a cooling fluid; a silicone member; a heatsink; a metal heatsink; a wire; and combinations thereof.

The technology described herein, along with the attributes and attendant advantages thereof, will best be appreciated and understood in view of the following detailed description taken in conjunction with the accompanying drawings in which representative embodiments are described by way of example.

### BRIEF DESCRIPTION OF THE DRAWINGS

FIG. 1 is a side sectional view of a device including multiple light delivery elements, consistent with the present inventive concepts.

FIG. 1A is a cross sectional view of the shaft of the device of FIG. 1, consistent with the present inventive concepts.

FIG. 2 is a side sectional view of a device including light delivery elements comprising light scattering material, consistent with the present inventive concepts.

FIG. 2A is a sectional view of the shaft of the device of FIG. 2 at a location entering the patient's body, consistent with the present inventive concepts.

FIG. 3 is a side sectional view of a device including a light delivery element comprising light scattering material and a light delivery element comprising a lens, consistent with the present inventive concepts.

FIG. 4 is a side sectional view of an infusion pump including a transcutaneous cannula with a light delivery element, consistent with the present inventive concepts.

FIG. 4A is a magnified view of a portion of the shaft of the device of FIG. 4, consistent with the present inventive concepts.

FIG. 5 is a side sectional view of an implanted device including a light delivery element, consistent with the present inventive concepts.

FIG. 5A is a magnified view of a portion of the shaft of the device of FIG. 5, consistent with the present inventive concepts.

FIG. 6 is a side sectional view of a device including a rotating light delivery element, consistent with the present inventive concepts.

FIG. 7 is a side sectional view of a device for delivering phototherapy, consistent with the present inventive concepts.

FIG. 7A is a cross sectional view of the device of FIG. 7, consistent with the present inventive concepts

FIG. 8 is a chart depicting a hemoglobin dissociation curve, consistent with the present inventive concepts.

FIG. 9 is a series of charts and molecular structure drawings comparing chlorophyll and heme, consistent with the present inventive concepts.

FIG. 10 is a schematic view of an energy delivery device comprising multiple shafts and multiple energy delivery elements, consistent with the present inventive concepts,

FIG. 11 is a schematic view of an implantable energy delivery device comprising at least one energy delivery element.

FIG. 12 is a side sectional view of a device including a light delivery element positioned in a balloon, consistent with the present inventive concepts.

FIG. 13 is a schematic view of a device comprising an external portion including a power converter and a first connector and a catheter portion including a light delivery element and a mating, second connector, consistent with the present inventive concepts.

### DETAILED DESCRIPTION OF THE DRAWINGS

Reference will now be made in detail to the present embodiments of the technology, examples of which are illustrated in the accompanying drawings. The same or like reference numbers are used throughout the drawings to refer to the same or like parts.

The terminology used herein is for the purpose of describing particular embodiments and is not intended to be limiting of the inventive concepts. As used herein, the singular forms “a,” “an” and “the” are intended to include the plural forms as well, unless the context clearly indicates otherwise.

It will be further understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be understood that, although the terms first, second, third, etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on”, “attached”, “connected” or “coupled” to another element, it can be directly on or above, or connected or coupled to, the other element or intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

Spatially relative terms, such as “beneath,” “below,” “lower,” “above,” “upper” and the like may be used to describe an element and/or feature's relationship to another element(s) and/or feature(s) as, for example, illustrated in the figures. It will be understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g. rotated 90 degrees or at other orientations) and the spatially relative descriptors used herein interpreted accordingly.

The term “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

For example, it will be appreciated that all features set out in any of the claims (whether independent or dependent) can be combined in any given way.

The devices of the present inventive concepts are constructed and arranged to be inserted into a mammalian body, such as through an incision in the skin or through a natural body orifice. The devices can include one or more light delivery elements configured to provide a therapeutic benefit to a patient. In some embodiments, light can be delivered to reduce or prevent infection, such as by delivering light that has a bactericidal effect and/or prevents colonization by bacteria. Alternatively or additionally, the light delivery elements can be configured to treat at least one of: virus; fungi; or one or more parasites. Direction of delivered light may be shown by arrows included in the drawings. In some embodiments, the devices further include a functional element constructed and arranged to further prevent and/or reduce an infection. The functional element can comprise one or more of: an electromagnetic field delivery element; an electric current and/or electric potential delivery element; or an ultrasound transducer.

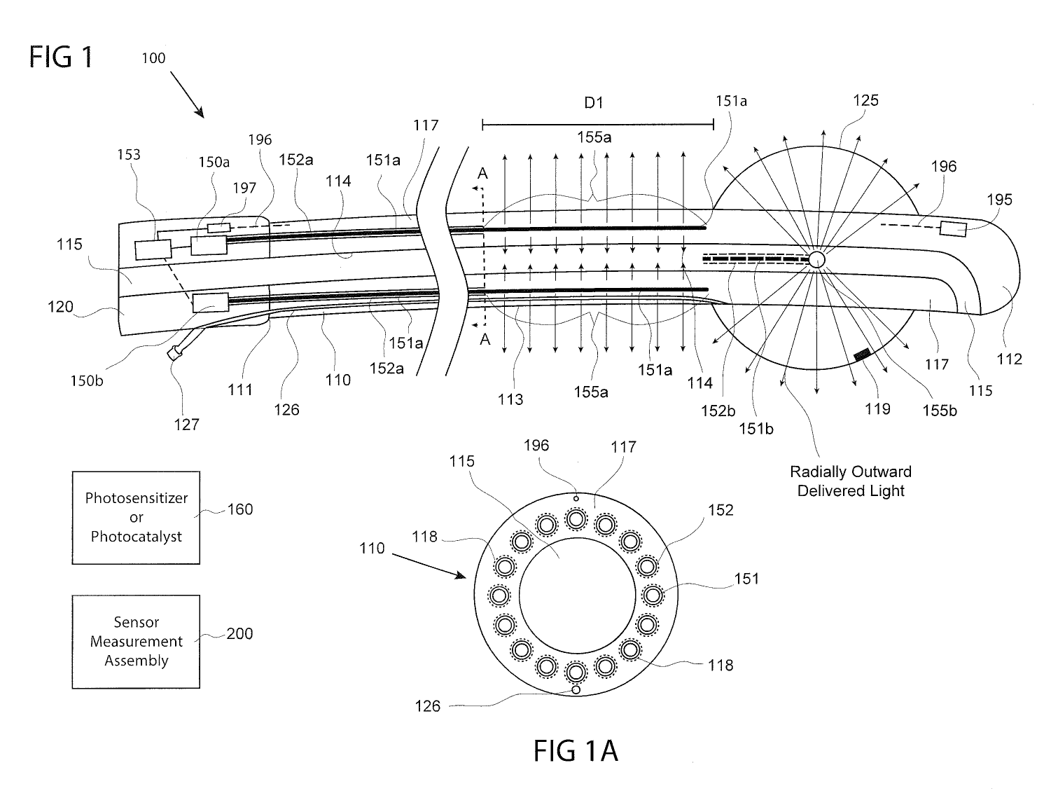

Referring now to FIG. 1, a side sectional view of a device with multiple light delivery elements is illustrated, consistent with the present inventive concepts. Device 100 includes shaft 110 comprising proximal end 111 and distal end 112, as well as outer surface 113. Proximal end 111 is attached to housing 120. Shaft 110 can be flexible or rigid, or it can contain both flexible and rigid portions. Shaft 110 can include one or more lumens, such as central lumen 115. Central lumen 115 is defined by inner surface 114 of shaft 110. A wall 117 of shaft 110 is positioned between inner surface 114 and outer surface 113. In some embodiments, shaft 110 includes multiple lumens, such as when device 100 comprises a multi-lumen catheter for insertion into a blood vessel or other body location. Shaft 110 can include a generally smooth outer surface 113, and can include a relatively uniform outer profile (at least in the distal portion) between 3 mm and 9 mm in diameter.

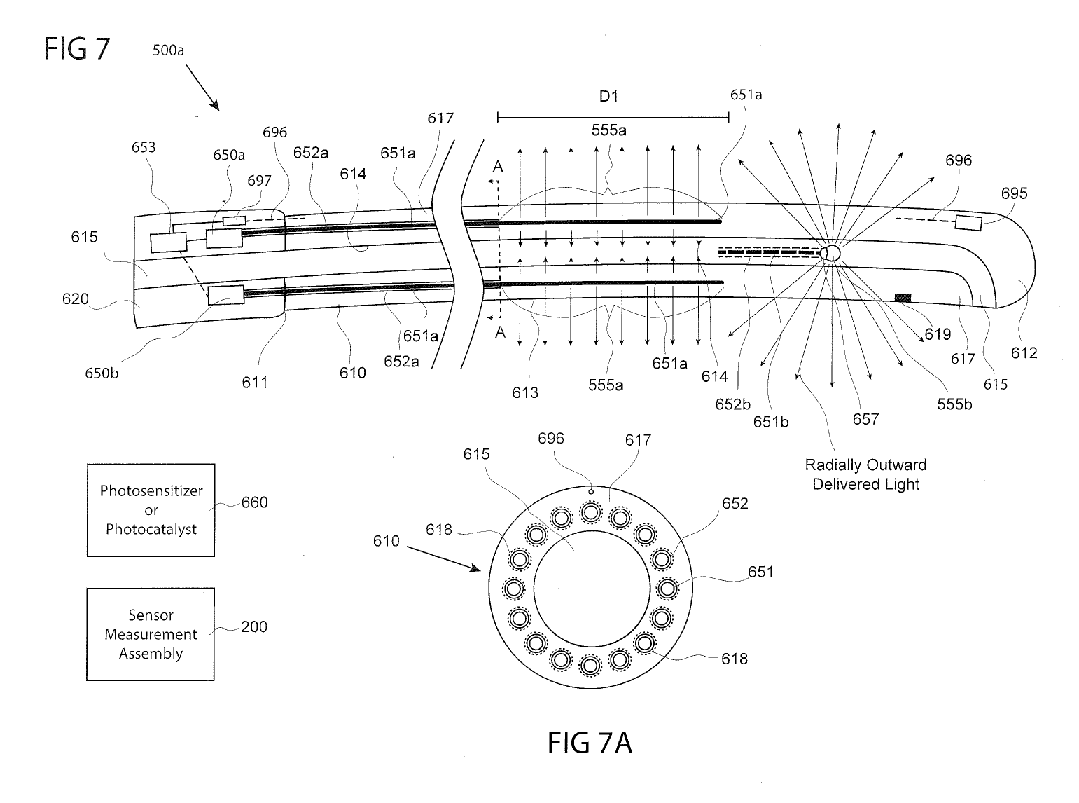

Shaft 110 can include one or more light delivery elements, such as one or more of light delivery elements 155a and 155b as shown, configured to deliver light such as to prevent and/or reduce infection. Alternatively or additionally, device 100 and/or light delivery elements 155a and/or 155b can be constructed and arranged to cause a physiologic effect selected from the group consisting of: blood temperature increase; vasodilation; increase in local nitric oxide; enhance nitric oxide release from the vascular endothelium; prolong local nitric oxide effects; alteration in the function of erythrocytes; modification in oxygen release from hemoglobin; modification in pH of blood; modulation in the immune response of blood leucocytes; modulation of the coagulation and/or thrombocyte function; modification in the function of heme catalyst enzymes in the blood; modification in hormonal action of a peptide and/or non-peptide hormone; modification in the binding capacity of one or more antibodies; decrease in blood glucose level; affect circulating tumor cells by selective heating, photocoagulation and/or photolysis; and combinations of these. In some embodiments, one or more of light delivery elements 155a and 155b are constructed and arranged to deliver phototherapy, such as is described herebelow in reference to FIGS. 7-11.

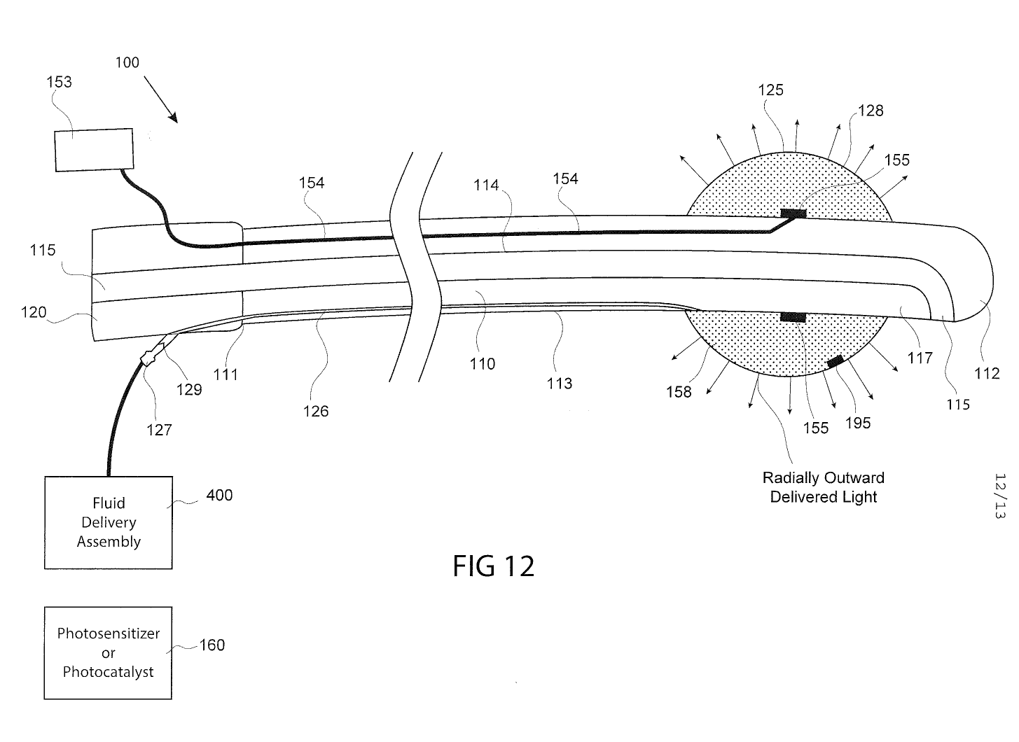

The light delivery elements of the present inventive concepts can comprise an element that both produces and delivers light (e.g. a light emitting diode or other light generating element) and/or these elements can simply deliver the light (e.g. a segment of exposed optical fiber or other optical element attached to a light source as described herein). The light delivery elements of the present inventive concepts can include one or more optical components, such as optical elements 157 described herein. An optical element can be constructed and arranged to couple light into a light delivery element, such as a lens or other optical element positioned to couple light from an optical fiber 151 (e.g. optical fiber 151a and/or 151b shown in FIG. 1, singly or collectively fiber 151) into a light delivery element 155. Alternatively or additionally, an optical element can be configured to distribute light from a light delivery element, such as a lens, prism or other optical element configured to distribute light in one or more desired patterns. Optical elements of the present inventive concepts can comprise an optical element selected from the group consisting of: lens; ball lens; prism; diffractor; filter; mirror; optical fiber; and combinations of these. In some embodiments, one or more light delivery and/or other energy delivery elements are positioned in a distal balloon, such as is described herebelow in reference to FIG. 12.

The cross section of FIG. 1 illustrates multiple light delivery elements 155 positioned within an axial segment of shaft 110, such as to deliver light radially out from and/or radially into shaft 110 (e.g. into a lumen 115 of shaft 110), as shown by the arrows emanating from each light delivery element 155a. Shaft 110 can include an anchoring and/or expanding element, such as balloon 125. An anchor, such as balloon 125 or other expandable element can be used to anchor shaft 110 at an internal body location such as the bladder. One or more light delivery elements 155b (one shown in FIG. 1) can be positioned within balloon 125 such as to transmit light radially out from balloon 125, as shown by the arrows emanating from light delivery element 155b and balloon 125. Light delivery elements 155a and 155b are operably connected to one or more sources of light, such as is described herebelow.

Device 100 can include one or more optical fibers, such as optical fibers 151a positioned within wall 117 of shaft 110. One or more fibers 151a can each be embedded in wall 117 of shaft 110. Alternatively or additionally, one or more fibers 151a can be insertable into (e.g. slidingly received by) a lumen, such as lumens 118 positioned within wall 117 (lumens 118 are omitted from FIG. 1 for illustrative clarity but shown in FIG. 1A and described herebelow). Alternatively or additionally, one or more fibers 151a can be positioned within central lumen 115. In some embodiments, central lumen 115 comprises multiple separate lumens and one or more optical fibers 151a can be positioned within and/or insertable into one or more of the multiple lumens of central lumen 115. Fibers 151a include an axial portion covered by an opaque covering, cladding 152a, such as the covered proximal portions of each fiber 151a shown in FIG. 1. Each light delivery element 155a comprises an axial portion of a fiber 151a that is not surrounded by an opaque material (e.g. the uncovered distal segments of each fiber 151a with length D1 shown in FIG. 1). Light introduced into the proximal end of each fiber 151a is conducted along the cladded optical fiber with minimal losses of light, as is known to those of skill in the art. However when reaching the uncladded portion defined by light delivery element 155a, the conducted light emanates radially out from the associated light delivery element 155a (e.g. a portion of fiber 151 that is not surrounded by cladding and can be modified to enhance the distribution of light), as shown by the arrows emanating from light delivery element 155a shown in FIG. 1. During use, light delivery element 155a can be positioned in an area of the patient to be treated (e.g. infection reduced or prevented), such as within a segment of the urethra.

Each light delivery element 155a can comprise a modified surface of fiber 151, such as a roughened surface and/or a surface otherwise modified to enhance the distribution of light. In some embodiments, one or more light delivery elements 155a can comprise a segment of optical fiber 151 that has received a surface treatment selected from the group consisting of: etching; cutting; covering with roughened material such as silicon; and combinations thereof. In some embodiments, light delivery element 155a comprises a segment of optical fiber 151 including at least a portion that is surrounded by a transparent covering. The transparent covering can include one or more reflective particles constructed and arranged to diverge the radiated light, such as one or more particles selected from the group consisting of: alumina particles; silica particles; titania particles; titanium oxide particles; and combinations of these.

Device 100 can include one or more optical fibers 151b extending from housing 120 to a distal portion of shaft 110, such as to a location within balloon 125. One or more optical fibers 151b can be positioned within lumen 115 and/or within wall 117 of shaft 110. Light delivery element 155b can comprise one or more optical elements (as described hereabove) positioned on the distal end of the one or more optical fibers 151b. In some embodiments, light delivery element 155b comprises a ball lens that is attached to the distal end of a fiber 151b or a ball lens that is formed from the distal end of fiber 151b. A ball lens can be created by melting the end of fiber 151b with applied heat. Alternatively, light delivery element 155b can comprise an uncovered distal portion (e.g. not covered by cladding 152b) as described hereabove in reference to light delivery element 155a.

Optical fibers 151a and/or 151b can comprise one or more materials configured to propagate the light for preventing and/or reducing infection. In some embodiments, fibers 151a and/or 151b comprise a material selected from the group consisting of: glass; plastic; polymethylmethacrylate (PMMA); one or more polymers (e.g. one or more polymers configured as a microstructured polymer optical fiber); photonic crystal; polycarbonate; polystyrene; and combinations of these. Fibers 151a and/or 151b can comprise flexible fibers which allow movement within the patient without breaking or other issues.

The proximal end of each fiber 151a is attached and/or attachable to a light source, such as light source 150a shown. The proximal end of each fiber 151b is attached and/or attachable to a light source, such as light source 150b shown. Device 100 further includes a battery, capacitor or other power source, such as power supply 153 shown positioned in housing 120 and operably connected to light sources 150a and 150b. In some embodiments, light source 150a and 150b (collectively light source 150) comprise a single light source 150 attached to one or more power supplies 153. Power supply 153 can comprise a replaceable or rechargeable power source, such as a rechargeable power source included when an implanted housing 120 surrounds power supply 153. In some embodiments, power supply 153 is positioned outside of housing 120, such as to prevent applying unnecessary forces to shaft 110 that might be uncomfortable to the patient.

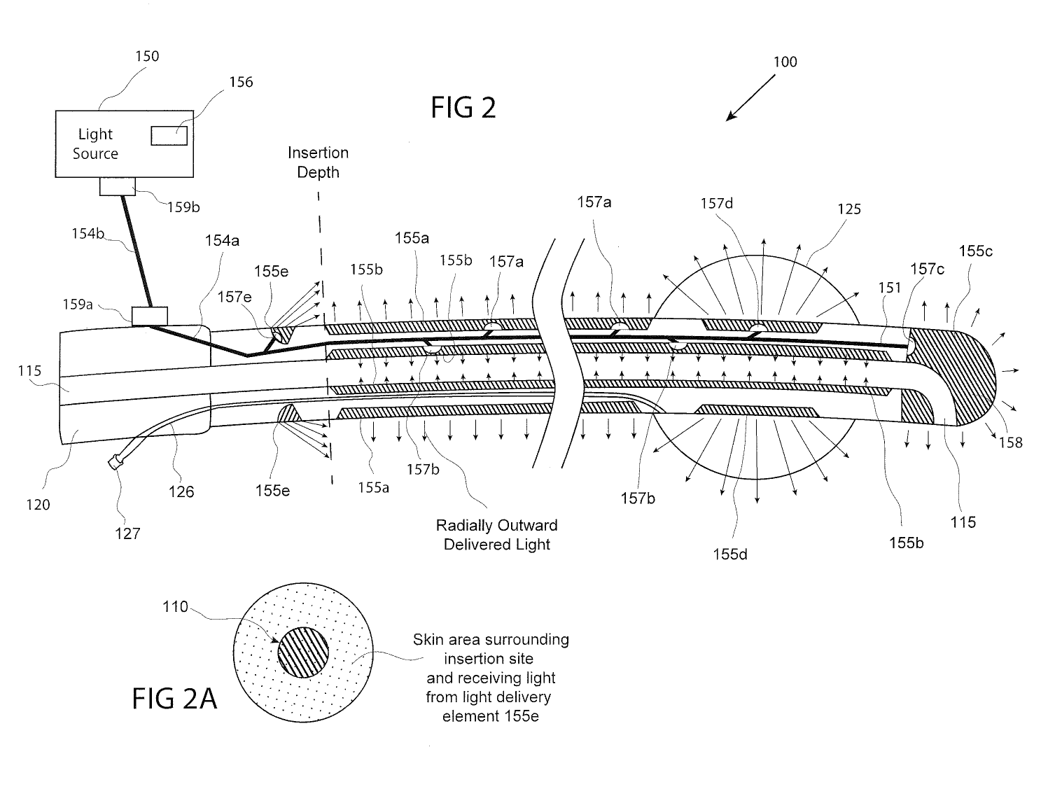

Light source 150 can be positioned within housing 120 (as shown in FIG. 1), on and/or within shaft 110 or balloon 125, or at an external location (as shown in FIG. 2). Light source 150 can be constructed and arranged to provide light at a single wavelength or multiple wavelengths of light simultaneously or pulsed to one or more light delivery elements 155, such as via one or more optical fibers 151 or other light carrying conduit. Light source 150 can comprise an LED, such as an organic LED. In some embodiments, a light delivery element 155 and light source 150 are positioned proximate each other, are attached to each other, and/or comprise the same component. In some embodiments, light delivery element 155b comprises light source 150 which can comprise an LED, lamp, laser, or other light source positioned within balloon 125.

Light source 150 can comprise a laser, such as a diode laser or other laser configured to provide one or more wavelengths of light. Light source 150 can be constructed and arranged to deliver pulsed light, such as light delivered at a duty cycle between 0.1% and 50%. Light source 150 can provide light at a power of less than 100 Watts, or less than 50 Watts. In some embodiments, light source 150 provides light at less than or equal to 100 Watts that is pulse-width modulated to deliver light at less than 50 Watts rms, or less than 20 Watts rms, to one or more light delivery elements 155. In some embodiments, light source 150 provides light at a power of at least 0.1 Watts, such as at a power between 2 Watts and 10 Watts. In some embodiments, light source 150 is configured to deliver light at a power of between 1.0 mW and 100 mW. In some embodiments, the light delivered by one or more light delivery elements 155 is delivered at a power between 1.0 mW and 100 mW.

Light source 150 can be constructed and arranged to provide one or more wavelengths of light between 300 nanometers and 900 nanometers, such as between 400 nanometers and 750 nanometers, or between 400 and 430 nanometers. Alternatively or additionally, light source 150 can be constructed and arranged to provide visible light and/or ultraviolet light. Light source 150 can be constructed and arranged to deliver light at varying wavelengths, such as light provided at continuously varying wavelengths or light alternative between at least a first wavelength and a second wavelength. Light source 150 can be constructed and arranged to provide light at multiple wavelengths simultaneously. In some embodiments, multiple wavelengths are provided by light source 150 to enhance the bactericidal effects of device 100, such as to effect multiple forms of bacteria, such as to reduce or eliminate one or more of: Escherichia coli; Klebsiella; Pseudomonas and other gram negative intestinal bacteria; Staphylococcus aureus; Streptococcus; a skin bacteria; Pneumococcus; Hämophilus; a respiratory tract bacteria; or anaerobic bacteria. In some embodiments, multiple wavelengths are produced or otherwise provided by light source 150 to reduce or prevent an infection resulting from one or more of: a virus; a fungus; or a parasite (e.g. malaria). In some embodiments, one or more wavelengths delivered by light delivery elements 155 are based on the absorption spectra of one or more microorganisms to be treated. For example, a wavelength of approximately 405 nm can be delivered to treat E. Coli; a wavelength of 470 nm can be delivered to treat staphylococcus; and/or a wavelength of 670 nm can be delivered to treat oral candida.

Light source 150 and/or one or more light delivery elements 155 can be constructed and arranged to provide light to an area (e.g. an area of tissue or urine) at a power density less than 500 mW/cm², such as at a power density less than 250 mW/cm², less than 100 mW/cm², or less than 10 mW/cm². In some embodiments, light source 150 and/or one or more light delivery elements 155 are constructed and arranged to deliver light at approximately 100 mW/cm². In some embodiments, device 100 is constructed and arranged to deliver light to tissue at a power density of between 1.0 mW/cm² and 10 mW/cm². In some embodiments, light source 150 and/or one or more light delivery elements 155 are constructed and arranged to deliver light at a level to prevent mucosal dehydration, such as pulse-width modulated light delivery configured to prevent mucosal dehydration.

Heating of the surrounding tissue and/or fluids (e.g. urine) can be monitored by one or more sensors of device 100, such as a temperature sensor 119. Temperature sensor 119 can be positioned in or proximate balloon 125 as shown, on or in shaft 110, or at another device 100 location. Temperature sensor 119 can be operably attached to, or operably attachable to, sensor measurement assembly 200, such as via one or more conduits, not shown but typically including one or more wires or optical fibers. In some embodiments, temperature sensor 119 comprises a thermocouple or thermistor attached to assembly 200 with one or more wires such that sensor measurement assembly 200 can produce a temperature measurement used to regulate the light provided by light source 150. In some embodiments, temperature sensor 119 can comprise one or more optical fibers (e.g. one or more optical fibers 151), which collect infrared light (e.g. from their distal end). In these embodiments, sensor measurement assembly 200 comprises an infrared sensor configured to correlate the received light to a temperature. The temperature determined by assembly 200 (e.g. via thermocouple signal, thermisters signal, infrared signal or otherwise), can be used to regulate the light delivered by light source 150 in a closed loop fashion, such as to prevent or reduce mucosal dehydration and/or prevent other tissue damage. Measurement assembly 200 can include a microprocessor and/or other electronic circuitry to receive one or more signals from temperature sensor 119 to determine a measured temperature. Alternatively or additionally, sensor 119 can comprise a non-temperature measurement device, such as when measurement assembly 200 is configured to record and/or interpret non-temperature measurement signals from sensor 119.

The light delivery elements 155 of the present inventive concepts can be constructed and arranged to deliver infection reducing and/or preventing (e.g. bactericidal) light to tissue and/or body fluids, such as tissue and/or body fluids selected from the group consisting of: blood; bladder wall tissue; urethral wall tissue; urine; esophageal tissue; airway tissue; subcutaneous tissue; vascular wall tissue; cardiac valve tissue; cerebrospinal fluid; meningeal tissue; synovial fluid; and combinations of these. The light delivery elements 155 can direct light toward a skin incision, such as a skin incision through which shaft 110 passes. The light delivery elements 155 can directly light toward a body fluid such as urine. Device 100 can be constructed and arranged such that the light delivered by one or more light delivery elements 155 prevent, eliminate and/or reduces colonization of foreign material by bacteria, such as to prevent, eliminate and/or reduce a biofilm of bacteria. Alternatively or additionally, the light delivered by one or more light delivery elements can be configured to treat at least one of: a virus; a fungus or a parasite.

The devices 100 of the present inventive concepts can be constructed for short-term clinical use with the patient (e.g. use for less than 16 hours, less than 24 hours, less than 3 days or less than 7 days), or for long-term clinical use (e.g. use for at least 1 week, at least 1 month, at least 3 months or at least 6 months). The light delivery elements 155 of the present inventive concepts can be constructed and arranged to deliver light (e.g. in a continuous or pulsed, intermittent manner) to reduce and/or prevent infection for short durations of time (e.g. less than 30 minutes, less than 1 hour, less than 4 hours, less than 16 hours, less than 24 hours, less than 3 days or less than 7 days), or for long-term use (e.g. at least 1 week, at least 1 month or at least 3 months). In some embodiments, one or more light delivery elements 155 are constructed and arranged to deliver light for or at least 6 months, such as when the one or more light delivery elements 155 are implanted in the patient. The light delivery elements 155 of the present inventive concepts can be constructed and arranged to be implanted within the patient, remain outside the patient's skin, or pass through the patient's skin via an incision or natural body orifice.

Light delivery elements 155a can be constructed and arranged to deliver light radially out from all or a portion of outer surface 113 of shaft 110, such as a majority portion and/or distal portion of the outer surface 113. Alternatively or additionally, light delivery elements 155a can be constructed and arranged to deliver light radially in from all or a portion of inner surface 114 of shaft 110, such as a majority portion and/or distal portion of the inner surface 114. In some embodiments, a light delivery element 155 is positioned to direct light toward a skin surface such as a skin surface surrounding a natural opening such as the skin surface surrounding the urethral orifice, or to a skin surface surrounding a skin incision and/or skin penetration site. In some embodiments, a light delivery element 155 comprises one or more optical components (e.g. a lens) configured to distribute light to a skin surface, not shown but such as is described in reference to FIG. 2 herebelow in reference to light delivery element 155d.

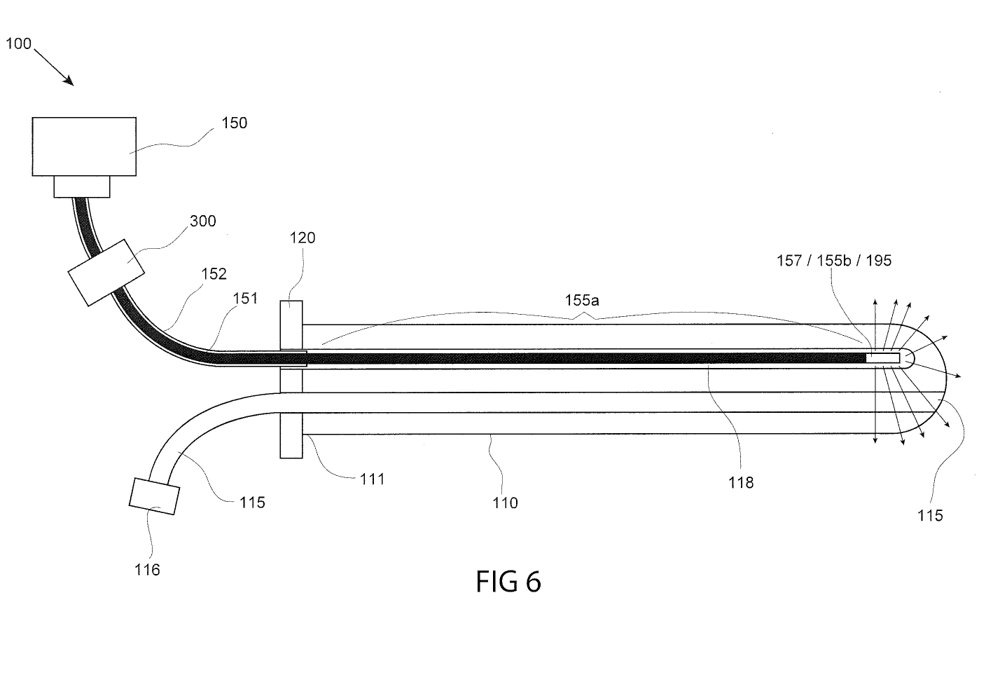

The light delivery elements 155 of the present inventive concepts can comprise one or more optical elements. In some embodiments, a light delivery element 155 can comprise an optical element selected from the group consisting of: optical fiber; lens; ball lens; prism; diffractor; filter; mirror; and combinations of these. An optical element can be positioned at the end of an optical fiber, such as a light delivery element 155b comprising a ball lens positioned at the end of fiber 151b, as described hereabove. In some embodiments, one or more light delivery elements 155 comprise light scattering material, not shown but such as is described in detail in reference to FIG. 2 herebelow. The light scattering material can comprise a material distributed relatively evenly throughout a silicone or other polymer material, such as a light scattering material selected from the group consisting of: alumina particles; silica particles; titania particles; titanium oxide particles; and combinations of these. In some embodiments, device 100 can be constructed and arranged to rotate one or more light delivery elements 155 (or a portion of a light delivery element 155), such as to distribute light over a larger area, rotating assembly not shown but described in detail in reference to FIG. 6 herebelow. One or more light delivery elements 155 can be constructed and arranged to deliver light to a majority of a cavity, such as a lens, prism, diffractor and/or mirror configured to direct light toward the majority of the surface of the bladder and/or the majority of a segment of a blood vessel wall. In some embodiments, one or more fibers 151 terminate within wall 117 of shaft 110, such as when the material of shaft 110 has a similar refraction index as fiber 151. In some embodiments, shaft 110 comprises light scattering material (e.g. titanium dioxide, TiO₂ particles) as described herein. In some embodiments, balloon 125 is filled with light scattering material (e.g. titanium dioxide particles) in water or other fluid, such that light delivered by one or more fibers 151 can be reflected in all directions relatively equally. In some embodiments, one or more portions of device 100, e.g. one or more portions of the outer surface of shaft 110 comprise a light dispersing coating (e.g. a titanium dioxide coating) which may be included with a photocatalyst. Titanium dioxide and/or another light dispersing component can be constructed and arranged to enhance hydrolysis, such as to generate oxygen radicals that enhance the bactericidal effects of the light delivered by device 100. In some embodiments, device 100 includes vanadium pentoxide as a photocatalyst.

The light delivery elements 155, shaft 110 and/or another component of device 100 can comprise a fluorescent material, such as a material constructed and arranged to increase light dispersion. In some embodiments, the fluorescent material can be positioned at least on the outer surface 113 and/or inner surface 114 of shaft 110, such as a fluorescent coating placed upon surfaces 113 and/or 114 of shaft 110. Other coatings can be included on one or more components of device 100, such as a photosensitizer constructed and arranged to be activated by light (e.g. light delivered by a light delivery element 155) to cause a bactericidal reaction. Shaft 110 can include one or more portions that are transparent or at least translucent (hereinafter “translucent”) to one or more wavelengths of light transmitted by a light delivery element 155. Shaft 110 can include a translucent portion surrounding a light delivery element 155a comprising an unclad portion of an optical fiber 151a.

In some embodiments, device 100 can include an anchoring element or an expandable element, such as balloon 125. Balloon 125 can be constructed and arranged to anchor shaft 110 in an internal body location such as the bladder of the patient. Balloon 125 can be fluidly attached to inflation lumen 126 which travels proximally within shaft 110 and through housing 120 to terminate at inflation port 127. Inflation port 127 can comprise a luer or other attachment element configured to fluidly attach to a fluid delivery device such as a syringe or fluid pump, such that balloon 125 can be expanded and/or contracted such as by a user such as a clinician, nurse, patient family member or the patient.

Housing 120 can be configured as a handle for a user, such as a clinician or patient, to hold while using device 100. Housing 120 can be implantable or include an implantable portion. Housing 120 can comprise a first housing and a second housing separated from the first housing. Housing 120 can surround one or more components including but not limited to: power supplies such as batteries; agent reservoirs such as pharmaceutical agent reservoirs; pumping mechanisms; energy delivery circuitry such as cardiac pacing or defibrillating circuitry; electronic processing circuitry; electronic memory circuitry; and combinations of these.

In some embodiments, device 100 comprises a device selected from the group consisting of: urine removal catheter; vascular access device; central venous catheter; peripherally inserted central catheter such as a peripherally inserted venous catheter; cerebrospinal fluid catheter, ventriculoperitoneal shunt, insulin pump; implanted device; implanted drug or other agent delivery pump; pacemaker; drive shaft assembly for a cardiac assist device; inflow and/or outflow cannula for a cardiac assist devices; neurostimulator; artificial heart; drainage catheter; colostomy tube; and combinations of these.

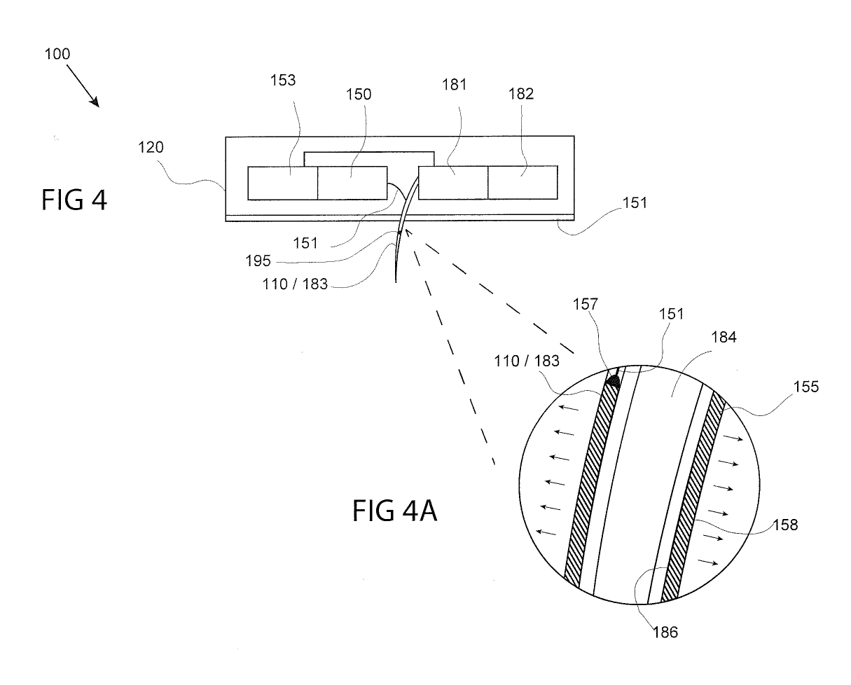

In some embodiments, shaft 110 is constructed and arranged for insertion through an incision of the skin, such as when shaft 110 is further inserted into a blood vessel, organ, and/or a subcutaneous tissue tunnel. In some embodiments, distal end 112 of shaft 110 is inserted into a blood vessel such that blood can be removed via lumen 115 and/or one or more agents can be delivered to an internal body location (e.g. into the cardiovascular system of the patient) via lumen 115. In these embodiments, housing 120 can include a luer or other attachment element configured to add or remove fluids via lumen 115, such as when device 100 comprises a central venous catheter or peripherally inserted central catheter. In some embodiments, shaft 110 is inserted through an incision in the skin and into the bladder, such as when device 100 is configured as a suprapubic bladder catheter. In some embodiments, shaft 110 is inserted through the skin and into an organ such as the kidney or into the ureter, such as when device 100 is configured as a urethral and/or nephrostomy catheter. In some embodiments, device 100 is configured as a colostomy tube inserted through the abdomen, such as to provide access to the intestine or colon. Alternatively, device 100 can comprise an external (i.e. non-implanted) drug delivery pump, such as a skin-attached or other external drug delivery device in which shaft 110 comprises a transcutaneous conduit (e.g. a rigid needle or flexible catheter passing through the skin surface into the subcutaneous tissue) for agent delivery. In these embodiments, housing 120 can include a pumping mechanism, not shown but described in reference to FIGS. 4 and 4A herebelow. In some embodiments, the agent delivered comprises an agent selected from the group consisting of: insulin; a chemotherapeutic agent; a nutritional material; a pain control agent such as morphine; and combinations thereof.

In some embodiments, shaft 110 is constructed and arranged for insertion into a natural body orifice, such as urethra; mouth; anus; vagina; nostril; ear hole; eye socket; and combinations of these. Device 100 can be constructed and arranged to prevent urinary tract infections. Shaft 110 can be constructed and arranged for insertion into the urethra and/or for insertion in the bladder, such as to support evacuation of urine from the patient. In some embodiments, one or more light delivery elements 155 are constructed and arranged to deliver light to a bladder, such as to a majority of the cavity of the bladder, such as to deliver light to the majority of urine in the bladder. Alternatively or additionally, one or more light delivery elements 155 can be constructed and arranged to deliver light to all or a portion of the urethra, such as to deliver light to all or a portion of the urine present in the urethra during light delivery. In some embodiments, light delivery element 155b is constructed and arranged to deliver light to a majority of the cavity of the bladder, and one or more light delivery elements 155a is constructed and arranged to deliver light to at least a distal portion of the urethra. The one or more light delivery elements 155a can be configured to deliver light to a segment of urethra of length D1 as shown. In some embodiments, D1 (the unclad portion of fiber 151a) is approximately 2 cm long, or at least 4 cm long, or at least 6 cm long, such that the distal 2 cm, 4 cm or 6 cm, respectively of the urethra receives light from light delivery elements 155a. In some embodiments, the total light delivered to the bladder is more than the total light delivered to the urethra. In some embodiments, the light delivered per area of the bladder and the urethra is approximately equal, such as at an amount less than or equal to 500 mW/cm², or less than or equal to 250 mW/cm², or less than or equal to 100 mW/cm². In some embodiments, device 100 is constructed and arranged to deliver light to tissue at a power density of between 1.0 mW/cm² and 10 mW/cm². The light provided by light source 150 and delivered by light delivery elements 155 can be constructed and arranged to have a bactericidal effect, such as to reduce or prevent bacterial colonization, such as to reduce and/or prevent infection. Alternatively or additionally, the light provided by light source 150 and delivered by light delivery elements 155 can be constructed and arranged to treat one or more of: a virus; a fungus or a parasite.

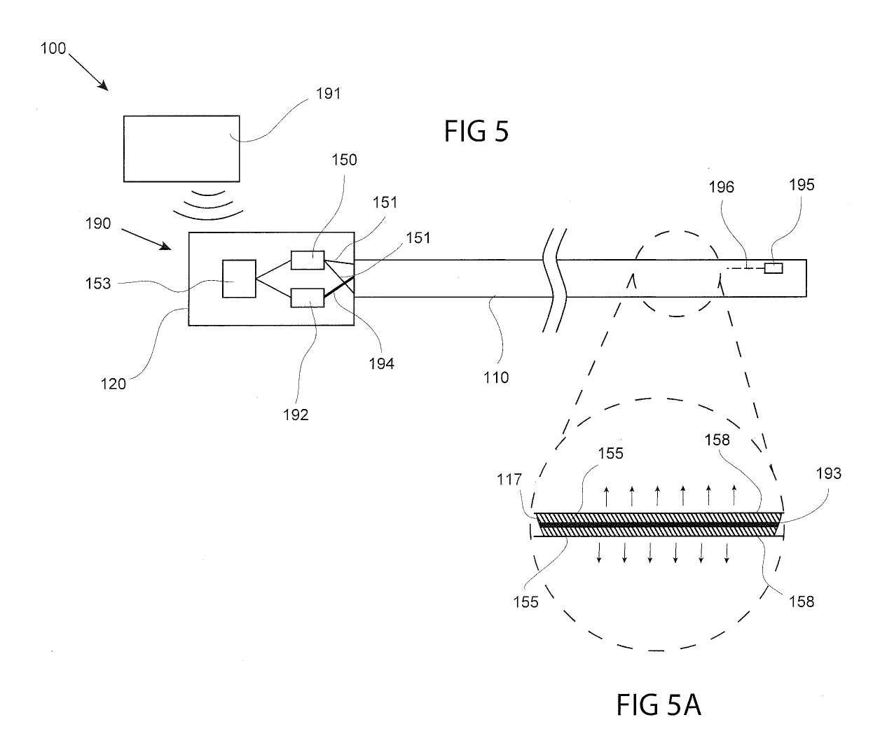

In some embodiments, shaft 110 and housing 120 are constructed and arranged for implantation into the patient, such that light delivery elements 155a and/or 155b can deliver light to an internal location within the patient to prevent and/or reduce infection at one or more locations surrounding shaft 110. Shaft 110 can be constructed and arranged for insertion through a subcutaneous tissue tunnel or other internal body location. Distal end 112 can be constructed and arranged for insertion into a blood vessel, a ventricle of the brain, a portion of the cerebrospinal fluid space, a joint capsule, a chamber of the heart, or other location, such as to provide a fluid conduit and/or to provide an electrical wire or optical fiber for a therapeutic application. Device 100 can comprise an implanted portion configured to deliver energy to one or more internal locations of a patient, such as to deliver energy to an organ such as a heart or brain. In these energy delivery embodiments, shaft 110 can include one or more conductors (e.g. within lumen 115) attached to an energy delivery unit within housing 120, conductors and energy delivery unit not shown but described in reference to FIGS. 5 and 5A herebelow. Alternatively or additionally, device 100 can comprise an implanted portion configured to deliver a pharmaceutical agent or other agent systemically and/or locally to an internal location of the patient, such as to deliver an agent systemically or locally within the patient. The delivered agent can be configured to improve the phototherapeutic effect of light delivered by one or more light delivery elements 155. In these embodiments, an implanted housing 120 can surround a drug reservoir and pumping means which deliver an agent to lumen 115 of shaft 110, drug reservoir and pumping means not shown but described in detail in reference to FIGS. 5 and 5A herebelow.

In some embodiments, device 100 further comprises a light enhancing material 160, such as a photosensitizer and/or a photocatalyst. The light enhancing material can be included in one or more components of device 100, such as a light enhancing coating applied to all or a portion of shaft 110 and/or balloon 125. The coating can be configured to dissolve or otherwise migrate from shaft 110 and/or balloon 125. The light enhancing material can be delivered by one or more components of device 100, such as through balloon 125 when balloon 125 comprise a porous balloon, from one or more openings in shaft 110 (e.g. lumen 115 at distal end 112) or from an outlet in another component of shaft 110. In some embodiments, the light enhancing material comprises a material selected from the group consisting of: toluidine blue O; methylene blue; and combinations thereof. In some embodiments, the light enhancing material is constructed and arranged to be activated by light delivered by device 100, such as light delivered by one or more light delivery elements 155.

One or more components of device 100 can be provided sterile, such as a sterile shaft 110. One or more components of device 100 can be reusable and/or re-sterilizable. In some embodiments, shaft 110 is used in a single use on a single patient, while light source 150 and/or one or more light delivery elements 155 (e.g. fibers 151) are reused in multiple uses with one or more patients (e.g. when light source 150 is operably attachable to fiber 151 and/or when fiber 151 is insertable into shaft 110).

In some embodiments, in addition to one or more light delivery elements 155, device 100 includes one or more additional components constructed and arranged to further prevent and/or further reduce infection, such as functional element 195 shown in FIG. 1. Functional element 195 can be constructed and arranged to improve upon the infection prevention and/or reduction than that which is achieved with the light delivered by the one or more light delivery elements 155 alone. Functional element 195 is connected to conduit 196, which can comprise one or more wires, optical fibers, or other energy carrying conduits. Conduit 196 travels proximally through shaft 110, and connects to a supply of energy, such as energy delivery unit 197. Energy delivery unit 197 can be contained within housing 120 (as shown), or at a location external to housing 120. Energy delivery unit 197 can be attached to power supply 153 or another supply of power via conduit 196. Conduit 196 can comprise at least two wires, such as two wires constructed and arranged to provide power to functional element 195 to create an electromagnetic field and/or an electric current (e.g. an electromagnetic field and/or electrical field provided to tissue and/or body fluid).

In some embodiments, functional element 195 comprises an element configured to deliver an electromagnetic field. Functional element 195 can produce one or more of: a dynamic electromagnetic field; a static electromagnetic field; a dynamic magnetic field; or a static magnetic field; a dynamic electrical field; or a static electrical field. Functional element 195 can produce a magnetic field with a field strength between 1 milliTesla (mT) and 500 mT. Functional element 195 can produce a magnetic field constructed and arranged to prevent adversely effecting one or more muscles and/or nerves. Alternatively or additionally, functional element 195 can comprise an element configured to deliver an electric current and/or electric potential. The delivered electric current and/or electric potential can be configured to cause electrolysis. Alternatively or additionally, functional element 195 can comprise an ultrasound transducer, and the produced ultrasound waves can be configured to further prevent or further reduce an infection. Energy delivery unit 197 provides electrical power, ultrasound signals, or other energy used to energize functional element 195.

Functional element 195 can be positioned on and/or in shaft 110, and/or within a location including balloon 125. In some embodiments, functional element 195 comprises one or more elements positioned on and/or within balloon 125, such that functional element 195 traverses radially from shaft 110 as balloon 125 is expanded, such as when balloon 125 is expanded within a bladder to anchor shaft 110 in the bladder. In some embodiments, functional element 195 is positioned along a majority of the length of shaft 110, or along a majority of the length of a portion of shaft 110 positioned under the patient's skin. In some embodiments, functional element 195 delivers an anti-infection effect (e.g. via delivery of an electric current and/or electric potential, electromagnetic field and/or ultrasound waves) to similar locations of tissue and/or body fluid to that receiving light from the one or more light delivery elements 155. In some embodiments, functional element 195 comprises multiple functional elements, such as multiple functional elements selected from the group consisting of: one or more electromagnetic field generating elements; one or more electric current and/or electric potential delivering elements; one or more ultrasound transducers; and combinations thereof. The multiple functional elements 195 can be, singly or in combination, constructed and arranged to at least one of further prevent or further reduce infection.

One or more functional elements 195 comprise one or more permanent magnets. In some embodiments, functional element 195 comprises multiple permanent magnets, such as multiple permanent magnets positioned on or in shaft 110. The multiple permanent magnets can be dispersed relatively uniformly along one or more portions of the length of shaft 110, such as along a full or partial circumferential portion of shaft 110, along a majority of the length of shaft 110 and/or along a majority of the length of shaft 110 which is inserted into the patient. In some embodiments, functional element 195 comprises multiple permanent magnets that are dispersed non-uniformly, such as when a higher density of magnets are positioned proximate to balloon 125 than along a more proximal portion of shaft 110.

In some embodiments, one or more functional elements 195 of device 100 are constructed and arranged to at least one of prevent or reduce infection, with or without the inclusion of one or more light delivery elements 155. In these embodiments, one or more light delivery elements 155 can be included to at least one of further prevent or further reduce infection.

Referring now to FIG. 1A, a cross sectional view of device 100 of FIG. 1 at line A-A is illustrated, consistent with the present inventive concepts. Shaft 110 includes wall 117 and central lumen 115. Positioned within wall 117 are an array of multiple optical fibers 151 (sixteen fibers 151 shown), such as optical fibers 151a or 151b of FIG. 1. Surrounding optical fibers 151 at section A-A is cladding 152. Cladding 152 is not included in the more distal portion of fibers 151 as described above. Wall 117 can include multiple lumens 118 configured to slidingly receive optical fibers 151. Also positioned within wall 117 is conduit 196 and inflation lumen 126, as described hereabove. In alternative embodiments, inflation lumen 126 comprises a lumen within an inflation tube positioned within lumen 115, such as an inflation tube well known to those of skill in the art.

Device 100 can be constructed and arranged to deliver light to one or more patient locations prior to any significant bacterial presence, such as to prevent initial biofilm formation and/or bacterial adhesion. Device 100 can be constructed and arranged to prevent emergence of drug resistant bacteria by avoiding biofilm creation (a known location for exchange of plasmids for drug resistance).

Referring now to FIG. 2, a side sectional view of a device with light delivery elements comprising light scattering material is illustrated, consistent with the present inventive concepts. Device 100 of FIG. 2 can include numerous components of similar construction, arrangement and function as similar reference numbered components of FIG. 1, such as shaft 110, central lumen 115, housing 120, balloon 125, inflation lumen 126 and inflation port 127. Device 100 includes multiple light delivery elements, 155a-e (collectively light delivery elements 155). Each of light delivering elements 155 can comprise a light scattering material 158 (depicted for light delivery element 155c only, for illustrative clarity but typically included in each of elements 155a-e). Light scattering material 158 can comprise a light scanning material embedded in a translucent material (e.g. a translucent polymer), and configured to relatively evenly distribute light delivered to each light delivery element 155. In some embodiments, the light scattering material 158 comprises a material selected from the group consisting of: alumina particles; silica particles; titania particles; and combinations of these.

Optically connected to each light delivery element 155 are one or more optical fibers 151 which collectively form optical fiber bundle 154a. Device 100 is configured such that light delivered by light source 150 travels into the one or more optical fibers 151 connected at their distal end to each light delivery element 155. Device 100 can include one or more optical fiber bundles and user attachable optical connectors configured to transmit light from light source 150 to optical fibers 151, such as fiber bundles 154a and 154b and connectors 159a and 159b shown in FIG. 2. In some embodiments, one or more of fiber bundles 154a and/or 154b (single or collectively fiber bundle 154 and/or bundle 154) can comprise one or more optical fibers, one or more wires and/or one or more other conduits. Light source 150 can include a user attachable connector 159b which can allow attachment of the proximal end of fiber bundle 154b to light source 150. Alternatively, fiber bundle 154b can be permanently attached to light source 150. Optical fibers 151 can collectively form fiber bundle 154a as shown. The proximal end of fiber bundle 154a can be configured to be operably attached to the distal end of fiber bundle 154b via a user attachable connector 159a, which can be attached to housing 120 (e.g. when housing 120 comprises a handle including connector 159a). Alternatively, fiber bundle 154a can be permanently attached to fiber bundle 154b or fiber bundle 154a and 154b can comprise a single, continuous fiber bundle without need of a connector. Light delivered by light source 150 travels through connector 159b, into fiber bundle 154b, through connector 159a and into fiber bundle 154a, and then into the individual optical fibers 151. In another alternatively embodiment, light source 150 is integral to housing 120 and fiber bundle 154a attaches directly to light source 150 without the need of connectors or additional wire bundles. In the embodiment shown in FIG. 2, light delivered by light source 150 travels through connector 159b, into fiber bundle 154b, through connector 159a and into fiber bundle 154a and then into the individual optical fibers 151 and eventually their attached light delivery elements 155.

Light source 150 can be constructed and arranged similar to light source 150 of FIG. 1, such as a light source configured to provide one or more wavelengths of light at one or more power levels as described hereabove. In some embodiments, light source 150 comprises one or more light emitting diodes, LED 156. In some embodiments, light source 150 comprises a laser.

Device 100 can include light delivery element 155a, which comprises the outer portion of an axial segment of shaft 110, such as a full circumferential outer segment of shaft 110 as shown. Light delivery element 155a is optically connected to one or more optical fibers 151. In some embodiments, optical element 157a (e.g. a lens or other optical component as described hereabove) is included to optically connect a fiber 151 to light delivery element 155a. Light provided by light source 150 is distributed radially out from shaft 110 (as shown by the arrows), at the location of light delivery element 155a, to prevent or reduce infection as described hereabove in reference to FIG. 1.

Device 100 can include light delivery element 155b, which comprises the inner portion of an axial segment of shaft 110, such as a full circumferential inner segment of shaft 110 as shown. Light delivery element 155b is optically connected to one or more optical fibers 151. In some embodiments, optical element 157b (e.g. a lens or other optical component as described hereabove) is included to optically connect a fiber 151 to light delivery element 155b. Light provided by light source 150 is distributed radially in from shaft 110 (e.g. into lumen 115 as shown by the arrows), at the location of light delivery element 155b, to prevent or reduce infection as described hereabove in reference to FIG. 1.

Device 100 can include light delivery element 155c, which comprises the distal tip portion of 110 as shown. Light delivery element 155c is optically connected to one or more optical fibers 151. In some embodiments, optical element 157c (e.g. a lens or other optical component as described hereabove) is included to optically connect a fiber 151 to light delivery element 155c. Light provided by light source 150 is distributed radially out from the distal tip of shaft 110 (as shown by the arrows), at the location of light delivery element 155c, to prevent or reduce infection as described hereabove in reference to FIG. 1.

Device 100 can include light delivery element 155d, which comprises an element positioned within balloon 125 as shown. Light delivery element 155d is optically connected to one or more optical fibers 151. In some embodiments, optical element 157d (e.g. a lens or other optical component as described hereabove) is included to optically connect a fiber 151 to light delivery element 155d. Light provided by light source 150 is distributed radially out from balloon 125 (as shown by the arrows), to prevent or reduce infection as described hereabove in reference to FIG. 1.

Device 100 can include light delivery element 155e, which comprises a light delivery element positioned on a circumferential outer segment of shaft 110 as shown. Light delivery element 155e is positioned at an axial location that is proximal to a typical insertion depth of device 100, such as a depth for insertion into a natural body orifice such as the urethra or a skin incision point. Light delivery element 155e is optically connected to one or more optical fibers 151. In some embodiments, optical element 157e (e.g. a lens or other optical component as described hereabove) is included to optically connect a fiber 151 to light delivery element 155c. Light provided by light source 150 is distributed toward a skin surface proximate the insertion depth location described above (as shown by the arrows of FIG. 2 and further illustrated in FIG. 2A), to prevent or reduce infection that can exist or attempt to form at the skin surface surrounding shaft 110.

In some embodiments, device 100 includes one or more additional components constructed and arranged to further prevent and/or further reduce infection, not shown but such as functional element 195 described in reference to FIG. 1 hereabove.

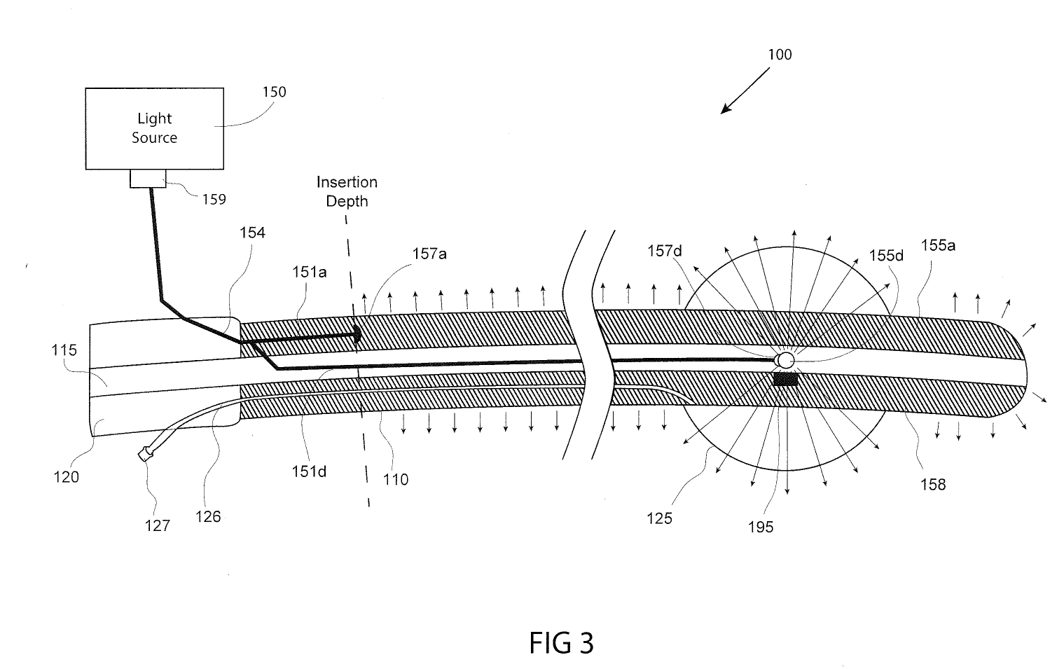

Referring now to FIG. 3, a side sectional view of a device with a light delivery element comprising light scattering material and a light delivery element comprising a lens is illustrated, consistent with the present inventive concepts. Device 100 of FIG. 3 can include numerous components of similar construction, arrangement and function as similar reference numbered components of FIG. 1, such as shaft 110, central lumen 115, housing 120, balloon 125, inflation lumen 126 and inflation port 127. In some embodiments, device 100 does not include balloon 125. In some embodiments, central lumen 115 comprises multiple separate lumens, such as two separate lumens used to deliver two different agents to the patient's cardiovascular system (e.g. a different agent in each lumen). Device 100 can include multiple light delivery elements, such as 155a and 155d shown (singly or collectively light delivery elements 155). Light delivery element 155a comprises a light scattering material 158. Light scattering material 158 can comprise a light scanning material embedded in a translucent material (e.g. a translucent silicone or other polymer), and configured to relatively evenly distribute light delivered to each light delivery element 155. In some embodiments, the light scattering material 158 comprises a material selected from the group consisting of: alumina particles; silica particles; titania particles; and combinations of these.

Optically connected to each light delivery element 155 are one or more optical fibers 151 which collectively form optical fiber bundle 154. Bundle 154 terminates in optical connector 159 which attaches to a separately housed light source 150. Alternatively, light source 150 can be integral to housing 120. Light source 150 can be constructed and arranged similar to light source 150 of FIG. 1, such as a light source configured to provide one or more wavelengths of light at one or more power levels as described hereabove. In some embodiments, light source 150 comprises one or more light emitting diodes. In some embodiments, light source 150 comprises a laser.

Device 100 can include light delivery element 155a, which comprises the outer portion of shaft 110, such as a full circumferential outer segment of all (as shown) or a portion of shaft 110. Light delivery element 155a is optically connected to one or more optical fibers 151a. In some embodiments, optical element 157a (e.g. a lens or other optical component as described hereabove) is included to optically connect a fiber 151a to light delivery element 155a. Light provided by light source 150 is distributed both radially out from shaft 110 (as shown by the arrows) and radially in toward lumen 115 (arrows not included for illustrative clarity), at the location of light delivery element 155a, to prevent or reduce infection as described hereabove in reference to FIG. 1.

Device 100 can include light delivery element 155d, which comprises an element positioned within balloon 125 as shown, such as an element comprising an optical element such as a ball lens or other lens, a prism, a diffractor, a mirror, and the like. Light delivery element 155d is optically connected to one or more optical fibers 151. In some embodiments, optical element 157d (e.g. a lens or other optical component as described hereabove) is included to optically connect a fiber 151 to light delivery element 155d. Light provided by light source 150 is distributed radially out from balloon 125 (as shown by the arrows) to prevent or reduce infection as described hereabove in reference to FIG. 1. In an alternative embodiment, optical element 157d comprises a light source, such as an LED, and optical fiber 151d can be replaced with a wire providing power to the LED.

In some embodiments, device 100 includes one or more additional components constructed and arranged to further prevent and/or further reduce infection, such as functional element 195. Functional element 195 can be constructed and arranged similar to functional element 195 described in reference to FIG. 1 hereabove, and device 100 can include conduit 196 and energy delivery unit 197, not shown in FIG. 3. In some embodiments, light source 150 comprises energy delivery unit 197. Functional element 195 can be positioned on and/or within one or more locations of shaft 110. Functional element 195 can be sized and positioned to deliver an anti-infection effect to similar locations of tissue and/or body fluid to that receiving light from the one or more light delivery elements 155a and/or 155d.

Referring now to FIG. 4, a side sectional view of an infusion pump comprising a transcutaneous cannula is illustrated, consistent with the present inventive concepts. Device 100 comprises housing 120 surrounding an agent delivery reservoir 182 fluidly attached to a pumping assembly 181. Device 100 includes a shaft 110 comprising a transcutaneous cannula 183 fluidly attached to pumping assembly 181 such that one or more agents contained with agent delivery reservoir 182 is propelled through cannula 183 by pumping assembly 181. Pumping assembly 181 can receive power from power supply 153 through the connection shown. Pumping assembly 181 can comprise a syringe pump, a peristaltic pump, a displacement pump, and the like. Cannula 183 can be constructed and arranged to be inserted through the patient's skin such that its distal tip is in an internal body location such as a blood vessel or in subcutaneous tissue. In some embodiments, cannula 183 is part of an attachable infusion set. In some embodiments, cannula 183 is injected into the skin via an injection mechanism internal to housing 120, injection mechanism not shown but such as known to those of skill in the art. Cannula 183 is constructed and arranged to prevent or reduce infection at locations proximate cannula 183.

Device 100 of FIG. 4 further includes a light source 150 and a power supply 153, such as have been described hereabove. Referring additionally to FIG. 4A, a magnified sectional view of cannula 183, cannula 183 comprises lumen 184. One or more optical fibers 151 optically couple light source 150 to a light delivery element 155 positioned on the outer surface of at least a portion of the length of the transcutaneous conduit, cannula 183. Light delivery element 155 can comprise an exposed (e.g. not covered by cladding material) portion of optical fiber 151. Alternatively, light delivery element 155 can be attached to optical fiber 151 as shown in FIG. 4A, such as via optical element 157 as has been described hereabove. Light delivery element 155 can comprise a translucent material, such as a translucent polymer, that is filled with light scattering material 158, also as has been described hereabove. In some embodiments, a reflecting element 186 can be positioned on the inner surface of light delivery element 155 such as to prevent light from being delivered toward lumen 184 of cannula 183. Reflecting element 186 can comprise a flexible reflecting surface or membrane, such as an element comprising a metallic or other reflecting material. Light emanating radially out from cannula 183 can be configured to prevent or reduce infection at locations proximate cannula 183, such as to prevent or reduction infection at a location proximate a skin penetration site through which cannula 183 is inserted.

In some embodiments, device 100 includes one or more additional components constructed and arranged to further prevent and/or further reduce infection, such as functional element 195. Functional element 195 can be constructed and arranged similar to functional element 195 described in reference to FIG. 1 hereabove, and device 100 can include conduit 196 and energy delivery unit 197, not shown in FIG. 4. In some embodiments, light source 150 and/or pumping assembly 181 comprise energy delivery unit 197. Functional element 195 can be positioned on and/or within one or more locations of shaft 110 (i.e. one or more locations along cannula 183). Functional element 195 can be sized and positioned to deliver an anti-infection effect to similar locations of tissue and/or body fluid to that receiving light from the one or more light delivery elements 155a.

Referring now to FIG. 5, an implantable device with a light delivery element is illustrated, consistent with the present inventive concepts. Device 100 includes an implanted portion 190 including housing 120. Housing 120 can surround a power supply 153, such as a rechargeable battery or capacitor, or other power supply element. Power supply 153 is operably attached to delivery unit 192 which in turn is operably attached to conduit 194. Device 100 further includes conduit 194, a flexible or rigid conduit which can include one or more electrical conductors, one or more optical fibers, and/or one or more lumens (e.g. for agent delivery). Conduit 194 is operably attached to shaft 110, such as an attachment to one or more conductors within shaft 110, to one or more optical fibers within shaft 110, and/or to one or more lumens with shaft 110. Delivery unit 192 and conduit 194 are constructed and arranged to deliver one or more agents and/or one or more forms of energy to shaft 110 to subsequently be delivered to an internal location of the patient. In some embodiments, delivery unit 192 and conduit 194 deliver tissue stimulating electrical and/or light energy to the patient. In these embodiments, device 100 can be configured to perform a function selected from the group consisting of: deliver pacing energy to the heart; delivery defibrillating energy to the heart; deliver electrical energy to the spine; deliver electrical energy to the brain such as to treat Parkinson's Disease, epilepsy or depression; deliver electrical energy to bone such as to stimulate bone growth; and combinations of these. Alternatively or additionally, delivery unit 192 and conduit 194 can delivery one or more agents, such as one or more pharmaceutical agents. In these embodiments, device 100 can be configured to perform a function selected from the group consisting of: deliver a pain control agent such as morphine; deliver an agent to the patient's spine such as to treat chronic pain; deliver a chemotherapeutic agent; deliver an agent to the patient's brain such as a neurological agent delivered to a ventricle of the brain; deliver one or more hormones to provide hormonal therapy to the patient; deliver insulin such as insulin delivered to the patient's cardiovascular system and/or subcutaneous tissue; and combinations of these.

Device 100 can include an external controller 191, such as a controller with a user interface used to transmit information to implanted portion 190 and/or receive information from implanted portion 190. External controller 191 can communicate with implanted portion 190 via one or more wireless communication means including radiofrequency telemetry; Bluetooth; infrared transmissions; and combinations of these. In some embodiments, controller 191 can be constructed and arranged to recharge a power supply of implanted portion 190, such as power supply 153, such as via inductive coupling of electromagnetic energy waves.

Referring additionally to FIG. 5A, device 100 further includes a light delivery element 155 along at least a portion of shaft 110, such as along at least a distal portion of shaft 110. In some embodiments, light delivery element 155 comprises a full circumferential segment of wall 117 of shaft 110. Light delivery element 155 can include including light scattering material 158. Alternatively or additionally, light delivery element 155 can include one or more exposed (e.g. not covered by cladding) segments of one or more optical fibers, such as one or more optical fibers 151. Positioned without housing 120 is light source 150, which is optically connected to at least one fiber 151. Fiber 151 is optically connected to light delivery element 155 directly, or through one or more optical elements, not shown but described hereabove in reference to component 157. Light delivered to light delivery element 155 travels through at least one fiber 151 and transmits at least radially out as shown by the arrows of FIG. 5A. In some embodiments, light is also transmitted radially inward (e.g. toward a lumen of shaft 110). Alternatively, light delivery element 155 can include a reflective element on its inner surface to prevent light being transmitted radially in, such as reflecting element 186 described hereabove.

In some embodiments, shaft 110 surrounds conductor 193, such as an electrical conductor or optical conductor constructed and arranged to deliver energy to tissue as described above (e.g. energy provided by delivery unit 192 and delivered via conduit 194). Alternatively or additionally, conductor 193 can include one or more lumens, such as one or more lumens constructed and arranged to deliver one or more agents to tissue, also as described above and delivered by delivery unit 192 via conduit 194.

In some embodiments, device 100 includes one or more additional components constructed and arranged to further prevent and/or further reduce infection, such as functional element 195. Functional element 195 can be constructed and arranged similar to functional element 195 described in reference to FIG. 1 hereabove, and device 100 can include conduit 196 (shown) and energy delivery unit 197 (not shown in FIG. 5). In some embodiments, light source 150 and/or delivery unit 192 comprise energy delivery unit 197. Functional element 195 can be positioned on and/or within one or more locations of shaft 110 (i.e. one or more locations along cannula 183). Functional element 195 can be sized and positioned to deliver an anti-infection effect to similar locations of tissue and/or body fluid to that receiving light from the one or more light delivery elements 155a.

Referring now to FIG. 6, a device with a rotating light delivery element is illustrated, consistent with the present inventive concepts. Device 100 of FIG. 6 can include numerous components of similar construction, arrangement and function as similar reference numbered components of FIG. 1, such as shaft 110, proximal end 111, lumen 115, housing 120, optical fiber 151, cladding 152, and light source 150. In some embodiments, device 100 of FIG. 6 comprises an expandable element or anchor, not shown but similar to balloon 125 of FIG. 1. Lumen 115 is fluidly attached to port 116, such as a luer connector, such that blood withdrawals and/or agent deliveries can be accomplished via lumen 115 when shaft 110 is inserted into a blood vessel or other internal body location as has been described in detail hereabove. In some embodiments, lumen 115 comprises two or more separate lumens, such as two or more lumens used to deliver two or more pharmaceutical or other agents while preventing mixing within device 100.

Shaft 110 includes lumen 118, which terminates within shaft 110 and can be configured to slidingly receive optical fiber 151 (e.g. when optical fiber 151 is inserted into a patient by a clinician, the patient or other user of device 100). The proximal portion of optical fiber 151 is surrounded by cladding 152. Light delivery element 155a comprises the distal, exposed portion of shaft 151, as shown. Light delivery element 155a can comprise a treated portion of optical fiber 151, such as an optical fiber 151 segment that has received a surface treatment as described hereabove. Alternatively or additionally, light delivery element 155b can be included, comprising optical element 157 shown positioned at the distal end of fiber 151. In some embodiments, cladding can cover the full length of fiber 151, such that light is delivered radially out solely by light delivery element 155b (i.e. by optical element 157).

The proximal end of optical fiber 151 is attached and/or attachable to light source 150. In some embodiments, shaft 110 comprises a disposable (i.e. single patient use) component and optical fiber 151, optical component 157 (if included) and light source 150 are reusable, such as to be reused with multiple shafts 110 after each has been inserted into an internal body location of the patient. Alternatively, shaft 110, optical fiber 151 and optical component 157 (if included) can be disposable and light source 150 be reused with multiple optical fibers 151.

In some embodiments, device 100 further comprises rotating assembly 300 which frictionally engages fiber 151 to cause full or partial rotations of fiber 151. Rotating assembly 300 can be configured to continuously rotate optical fiber 151, such as continuous uni-directional 360° rotations, or to rotate optical fiber 151 in discrete or reciprocating (back and forth) motions. Rotation of fiber 151 can be used to rotate optical element 157, such as to circumferentially distribute light delivered by optical element 157. In some embodiments, light delivery element 155a comprises a segment of optical fiber 151 that delivers light in an asymmetric pattern (e.g. due to an asymmetric coating or surface treatment), and rotation of fiber 151 can be used to deliver light in an even circumferential pattern.

In some embodiments, device 100 includes one or more additional components constructed and arranged to further prevent and/or further reduce infection, such as functional element 195. Functional element 195 can be constructed and arranged similar to functional element 195 described in reference to FIG. 1 hereabove, and device 100 can include conduit 196 and energy delivery unit 197 (neither shown in FIG. 6). In some embodiments, light source 150 comprises energy delivery unit 197. Functional element 195 can be positioned on and/or within one or more locations of shaft 110, such as when light delivery element 155b comprises functional element 195 as shown in FIG. 6. Functional element 195 can be sized and positioned to deliver an anti-infection effect to similar locations of tissue and/or body fluid to that receiving light from the one or more light delivery elements 155a.

FIGS. 7 through 11 illustrate devices and methods for delivery of light or other electromagnetic energy to provide a therapeutic benefit, such as to treat: heart failure; kidney failure; liver failure; transplanted organ failure; cancer; autoimmune disease; inflammatory reactions; and/or cerebral disease. Devices 500a, 500b and 500c of FIGS. 7, 10 and 11, respectively, collectively devices 500, each include one or more energy delivery elements 555. Energy delivery elements 555 can be constructed and arranged to deliver one or more forms of energy, such as electromagnetic energy or other energy. In some embodiments, one or more energy delivery elements 555 are configured to deliver energy selected from the group consisting of: light energy; electrostatic field energy; magnetic field energy; varying field energy; microwave energy; ultrasound energy; and combinations of these. In some embodiments, one or more energy delivery elements 555 are constructed and arranged to deliver light energy and a second form of energy, such as electrostatic field energy; magnetic field energy; and/or electromagnetic field energy.

In some embodiments, one or more energy delivery elements 555 are constructed and arranged to deliver at least light energy, such as light energy comprising one or more wavelengths, such as one or more wavelengths between 300 nm and 900 nm, such as between 250 nm and 730 nm (e.g. to affect nitric oxide), between 410 nm and 580 nm, between 410 nm and 420 nm, between 540 nm and 550 nm, or between 570 nm and 580 nm. In some embodiments, energy delivery elements 555 are constructed and arranged to deliver visible light, ultraviolet light and/or infrared light. In some embodiments, energy delivery elements 555 deliver one or more wavelengths of light constructed and arranged to cause immunomodulation. Device 500 and/or energy delivery elements 555 can be constructed and arranged to deliver phototherapy and/or thermal therapy, such as be delivering electromagnetic waves to blood or other tissue. The delivered electromagnetic waves can be configured to alter the function of heme proteins and/or enzymes to effect metabolic, vascular and/or immunomodulative function.

One or more energy delivery elements 555 can comprise an LED constructed of an organic material. Energy delivery elements 555 can each comprise a phosphorescent material, such as one or more polymers such as poly(n-vinylcarbazole) used as a host to which an organometallic complex (e.g. an iridium complex) is added as a dopant (e.g. to increase energy efficiency).

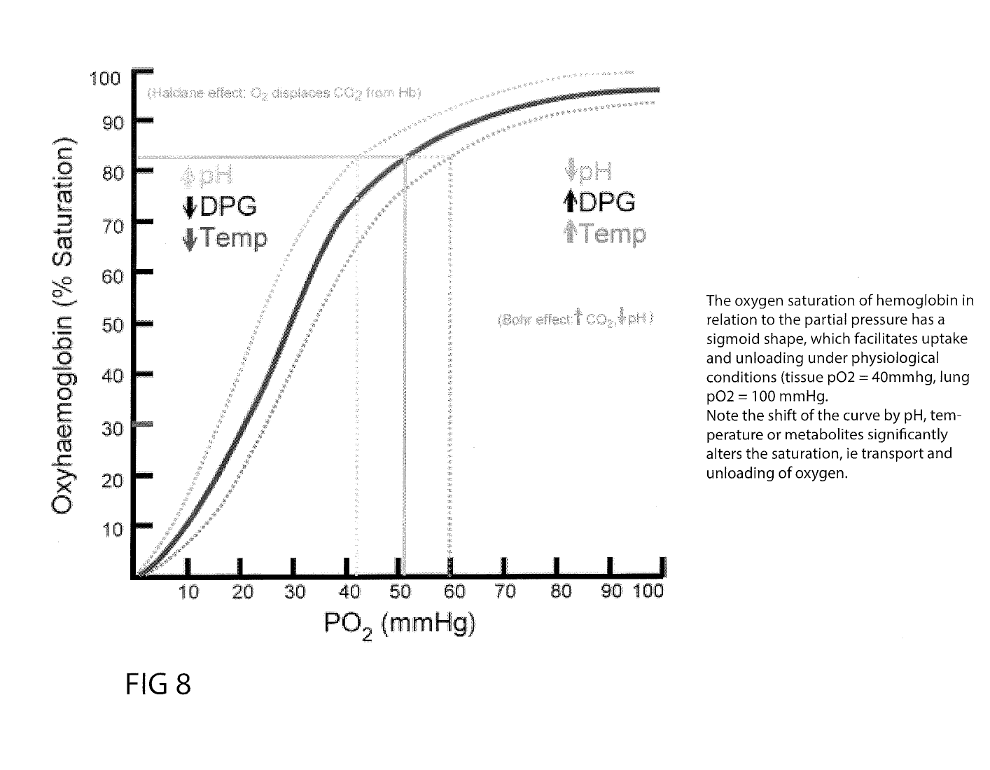

Energy delivery elements 555 can be constructed and arranged to warm the patient's blood, such as via the delivery of microwave or ultrasound energy, such as to modify oxygen unloading as seen in the dissociation curve of FIG. 8. In some embodiments, energy delivery elements 555 are constructed and arranged to both warm blood and deliver light energy to blood.

One or more energy delivery elements 555 can be constructed and arranged to deliver energy in a continuous manner, and/or in a pulsed, intermittent manner. Device 500 and one or more energy delivery elements 555 can be constructed and arranged to deliver energy in a closed-loop fashion, such as in response to a signal provided by a sensor, such as sensor 619, 719 and 819 of FIGS. 7, 10 and 11, respectively, each described herebelow. Energy delivery can comprise light energy or other electromagnetic energy that is adjusted based on a sensor signal. In some embodiments, an electromagnetic field strength is adjusted based on the sensor signal. In some embodiments and pulse-width modulation parameter (e.g. ratio of on time to off time) is adjusted based on the sensor signal. Sensors of the present inventive concepts such as sensor 619, 719 and 819 can comprise a sensor selected from the group consisting of: temperature sensor such as a thermocouple or thermister; oxygen sensor; glucose sensor such as an optical glucose sensor; pH sensor; physiologic sensor; pressure sensor; blood gas sensor; blood conductivity sensor; impedance sensor; motion sensor; accelerometer; and combinations of these. In some embodiments, energy delivery is modified based on the conductivity and/or impedance of blood. In some embodiments, energy delivery is modified based on a measured blood glucose level, such as a blood glucose level measured by an optical glucose sensor.

Devices 500 of the present inventive concepts can be constructed for short-term clinical use with the patient (e.g. use for less than 16 hours, less than 24 hours, less than 3 days or less than 7 days), or for long-term clinical use (e.g. use for at least 1 week, at least 1 month, at least 3 months or at least 6 months). The energy delivery elements 555 of the present inventive concepts can be constructed and arranged to deliver light (e.g. in a continuous or pulsed, intermittent manner) to provide phototherapy and/or thermal therapy for short durations of time (e.g. less than 30 minutes, less than 1 hour, less than 4 hours, less than 16 hours, less than 24 hours, less than 3 days or less than 7 days), or for long-term use (e.g. at least 1 week, at least 1 month or at least 3 months). In some embodiments, one or more energy delivery elements 555 are constructed and arranged to deliver light for or at least 6 months, such as when the one or more energy delivery elements 555 are implanted in the patient. The energy delivery elements 555 of the present inventive concepts can be constructed and arranged to be implanted within the patient, remain outside the patient's skin, or pass through the patient's skin via an incision or natural body orifice.

Devices 500 of FIGS. 7, 10 and 111 can include a photosensitizer or other agent, such as agent 660 of FIG. 7. Agent 660 can be a patient ingested or otherwise delivered to the patient and enhances the effect of the phototherapy, such as to enhance or otherwise modify the absorption of light by blood or other tissue of the patient. Agent 660 can be given to the patient to enhance target enzyme effects, such as when agent 660 comprises a nitric oxide donor medication, tetrahydrofolate, and/or other substrates for heme enzymes.

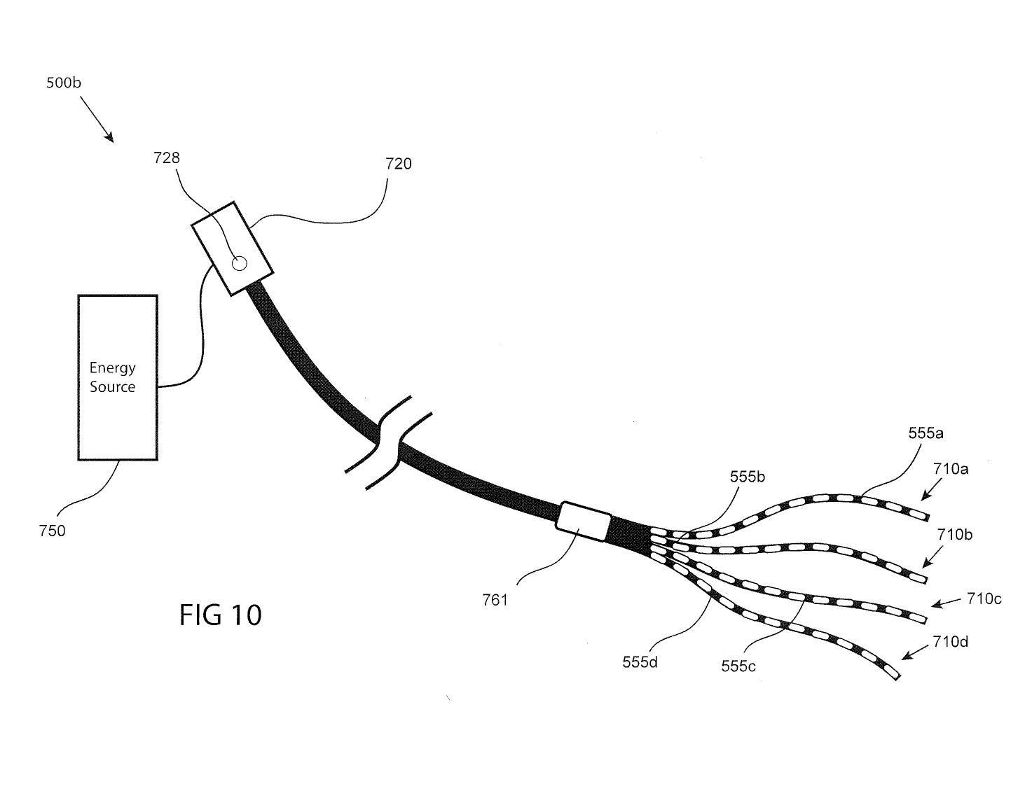

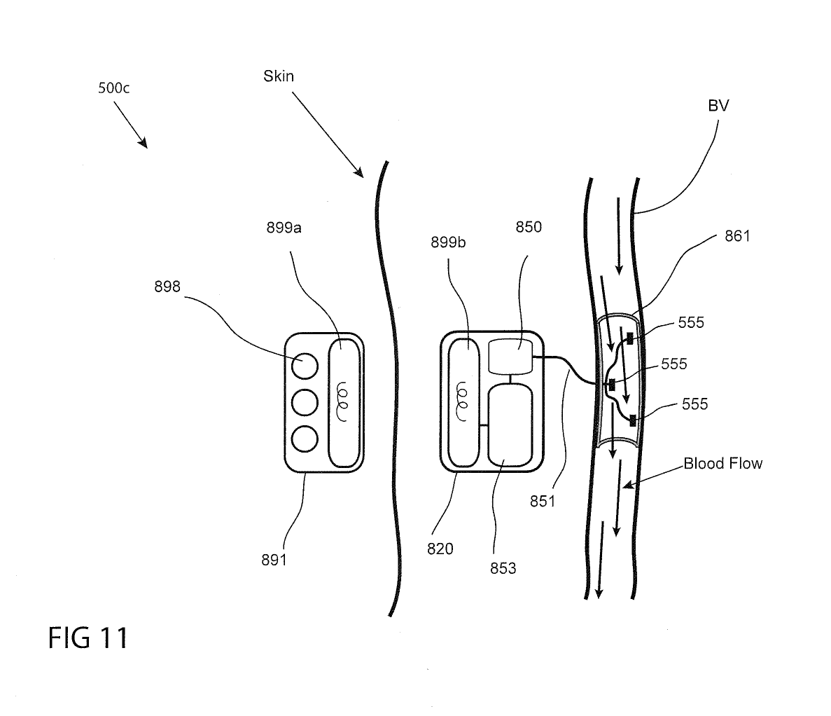

Device 500a of FIG. 7 includes a shaft, shaft 610, configured to be inserted into the patient, such as through a natural orifice or percutaneous incision to access a blood vessel. Device 500b of FIG. 10 includes multiple shafts, shafts 710, that can be positioned at multiple locations, such as in multiple blood vessels, such as in multiple pulmonary arteries. Device 500c of FIG. 11 comprises an implantable device from which one or more shafts 810 are attached and can be placed to one or more internal body locations. One or more energy delivery element 555 can be positioned on, in and/or within one or more shafts such as shafts 610, 710 and 810, such as one or more energy delivery elements 555 positioned on the distal end of the shaft, in the distal portion of the shaft, or alone a majority of the length of the shaft. In some embodiments, one or more energy delivery elements 555 can be advanceable from a shaft, such as an energy delivery element 555 mounted to a control rod, not shown but configured to advance one or more energy delivery elements 555 from the side of a shaft or from the distal end of a shaft. In some embodiments, one or more shafts include a coating, such as an anti-thrombogenic coating such as a surface comprising nanoparticles configured to carry a negative electrical charge.

Heart failure is a condition, where the pumping function of the heart is insufficient to deliver adequate amount of blood for oxygen delivery to vital organs. Oxygen transport is the main function of cardiovascular circulation. At a constant cardiac output, the circulation to target organs and other body tissue locations depends critically on the degree of vascular resistance. In the case of tissue underperfusion (e.g. shock), vascular resistance is increased by vasoconstriction in order to increase blood pressure and thereby perfusion pressure. Further decrease of tissue perfusion by vasoconstriction can lead to a vicious circle aggravating oxygen depth. To improve oxygen delivery to tissue, one or more treatments can be implemented. An increase in cardiac output can be attempted, such as by administering one or more pharmaceutical agents or cardiac pacing. Alternatively or additionally, a therapy to decrease in peripheral vascular resistance can be employed. ACE inhibitors, angiotensin blockers and/or other cardiac medications have improved cardiac output and survival. Similarly nitric oxide has been shown to act as an endogenous vasodilator improving perfusion, especially in the pulmonary circulation. In cases of hypoxemia, local vasoconstriction, which is a physiologic response to decreased perfusion of poorly ventilated regions, increases the afterload for the right ventricle considerably in diseases such as: shock; pulmonary disease; scleroderma; primary pulmonary hypertension; and/or intracardiac shunt. However, due to the global site of action, the success of these non-specific medications is limited by overall blood pressure decline and thereby impairment of perfusion of critical organs (e.g. the kidneys and brain). In addition, specific vasodilator treatment for the pulmonary circulation without systemic effects is lacking, with the exception of inhaled nitric oxide. Although nitric oxide has been proven a potent vasodilator with great therapeutic potential, it is expensive and difficult to deliver. Therefore a method to locally increase availability of nitric oxide provides an attractive alternative.

Another method of increasing oxygen delivery to tissue can include improving oxygen unloading. Animals living at high altitude (e.g. birds and other mammals living in the Himalayan mountains) have mutations in their hemoglobin which has led to facilitated oxygen unloading and thereby improving tissue oxygenation. In humans, several humoral factors associated with metabolic demand, such as increased temperature, decreased pH and increased metabolites such as 2,3DPG facilitate oxygen unloading and thereby local oxygen delivery to tissues, as described herebelow.

Oxygen delivery to the body is determined by the cardiac output and the arterio-venous difference of oxygen content (Ficks law: oxygen consumption=cardiac output x arteriovenous oxygen difference). In disease such as heart failure, oxygen delivery to organs and other tissue is diminished because of reduced cardiac output. However, oxygen delivery can be improved by widening the arterio-venous difference of oxygen content. Better oxygen unloading from hemoglobin improves organ and other tissue oxygen availability and leads to lower venous oxygen content. The characteristics of hemoglobin oxygen unloading are summarized in the hemoglobin dissociation curve shown in FIG. 8.

The devices and methods of the present inventive concepts can be constructed and arranged to shift the hemoglobin dissociation curve to the right, such as to improve oxygen unloading and thereby improve tissue oxygen availability. These improvements can decrease the symptoms and consequences of heart failure such as impaired renal and other organ function. In some embodiments, the devices and methods of the present inventive concepts are constructed and arranged to cause a temporary, reversible, right shift of the hemoglobin dissociation curve in the precapillary lung, such as to treat pulmonary hypertension.

The devices and methods of the present inventive concepts can be constructed and arranged to enhance or otherwise modify one or more functions of blood, such as by delivering light, electrical current and/or other electromagnetic energy, such as to modify one or more of the blood functions described immediately herebelow.

The function of blood is to transport oxygen from the lungs to the tissue, which is accomplished by the red blood cells. Hemoglobin binds to oxygen. If the oxygen binding is decreased by shifting the oxygen dissociation curve to the right, increased amounts of oxygen are released to the tissue. This release occurs in cases of decreasing pH, increasing temperature, and in the presence of metabolites of glycolysis, such as DPG. Any artificial right shift of the oxygen dissociation curve by the alteration of blood pH, blood temperature and/or direct photochemical or electrochemical interaction with hemoglobin might therefore improve oxygen availability in the tissue and thereby alleviate the symptoms and consequences of heart failure.

Another function of the blood is transport of hormones, cytokines, antibodies and other messenger molecules between the organs or the site of production to the site of action. Any artificial modification of the chemical structure, conformation or electrical binding affinity of those messenger molecules can be used for therapeutic action, such as an increase or decrease of hormone effects (e.g. insulin, adrenalin) and/or inactivation or activation of circulating antibodies, cytokines, etc.

Another function of the blood is the immunologic function of the white blood cells, which is important for defense against infectious agents but is also responsible for a variety of unwanted reactions, for instance in autoimmune diseases, inflammatory diseases (e.g. arthritis) and inflammatory processes due to transplantation. Extracorporeal light therapy of blood alters its immune function after reinfusion. Device 500 can cause similar therapeutic effects to extracorporeal light therapy. For instance, device 500 can be placed in an artery supplying a transplanted organ (e.g. kidney, liver, heart, lung, intestine, pancreas, etc). and activated if rejection by the hose is anticipated. Device 500 can be placed in a central vessel such as the right atrium in order to modulate the circulating white blood cells and immune system, such as to treat graft versus host disease, autoimmune disease, chronic inflammatory diseases, allergic reactions and/or leukemia.

Another function of the blood is coagulation, which prevents bleeding. A cascade of clotting factors circulates in the blood and is activated readily in case of trauma and/or contact with an artificial surface or chemical agents. Unfortunately, clot formation can lead to embolus or undesired vascular occlusion. Clots can be dissolved by fibrinolysis, but the balance of the coagulation and fibrinolysis is very delicate. A therapeutic intervention with light therapy via device 500 can offer unique opportunities to treat disseminated intravascular coagulation disorders (DIC), as they occur in the setting of acute disease (bacteriemia, cancer) and coagulation associated with the implantation of foreign bodies such as stents. Thrombosis is a major complication of stent implantation.

Electromagnetic waves as light are absorbed by solid particles of the blood. However, if the wavelength of the electromagnetic wave delivered by device 500 is similar to the diameter of the erythrocytes (7-10 micrometer), then the penetration of the wave is sufficient for reaching the entire volume inside the blood vessel. The wavelength delivered by device 500 can range from visible light up to microwaves. The delivered wavelength can range up to 50 micrometers and longer. The wavelengths delivered by device 500 can be used in the circulatory system to view a vessel wall and/or treat the vessel wall by photodynamic therapy. The energy of the light can be deposited at the vessel wall and not within the blood.

As described herein, device 500 can be constructed and/or arranged to deliver light and electric current, such as an electric current constructed and arranged to cause electrolysis. In addition to light delivery, the electromagnetic energy emission and the application of current has a profound impact on pH of the blood and the electrochemical equilibrium. Electrolysis of water results in H₂ and O₂, however electrolysis in the presence of physiologic NaCl, as in blood, results in chloride, hydrogen and caustic soda lye (2NaCl+2H₂O->CL₂+H₂+2 NaOH). To prevent toxic effects, device 500 can include an ion exchange membrane around a location generating one or more toxic substances, membrane not shown but configured to prevent any toxic materials from entering the blood stream. Device 500 can be constructed and arranged to alter blood pH, oxygen availability and other electrochemical reactions, depending on the location of the two or more electrodes providing electrolysis (e.g. an anode and a cathode). Device 500 can be configured such that reactions take place at an electrode configured as an anode (e.g. electrons absorbed to generate H+ ions) and an electrode configured as a cathode (electrons emitted for example to generate H2). Alternatively or additionally, electrolysis can be achieved by photoactivation by device 500 of a catalyst such as titanium oxide.

Device 500 can be constructed and arranged to alter electromagnetic fields within the blood to change the conformation of proteins, such as coagulation factors, hormones and other messenger proteins and the entire spectrum of actions described hereabove.

One or more portions of device 500 (e.g. portions positioned within the body) may be treated with a biocompatible coating such as parylene.

Hematogenetic spread of cancer involves transport of cancer cells from the primary site to other organs by blood transport. The insertion of device 500 within one or multiple veins draining the area of a primary tumor might prevent the formation of hematogenous metastasis by electrochemically and/or photodynamically inactivating circulating cancer cells. For instance, microwaves with a wavelength which is larger than the diameter of an erythrocyte and leucocyte, but smaller than the cancer cell, will be absorbed by the cancer cell while it goes through the blood cells. Thereby a selective heating is possible which can be used to selectively inactivate cancer cells while preserving the blood cells.

In sensitizing of certain target cells (erythrocytes, leucocytes, cancer cells, etc.), the use of a photosensitizer and/or ferromagnetic particles, alone or in combination with specific antibodies, can enhance the therapeutic effect of the energy delivered by device 500. In particular, the use of optomagnetic particles containing Fe, Co, Ni or other magnetizable elements can enhance the therapeutic effect of phototherapy delivered by device 500. For example, the magnetization and/or demagnetization of hemoglobin ferrous ions can change oxygen affinity and can be used to improve tissue oxygenation. Hemoglobin occurs in three types, oxyhemoglobin (not magnetic), deoxyhemoglobin (magnetic) and methemoglobin (not magnetic). By device 500 applying a magnetic field, the deoxyhemoglobin can be stabilized and thereby the oxygen unloading of hemoglobin facilitated.

At least a portion of device 500 can be placed temporarily around and/or in a blood vessel such as a vein or an artery. Alternatively, at least a portion of device 500 can be implanted within the patient, such as an implanted portion comprising a power supply such as a battery or capacitor and recharging circuitry such as an inductive coupling recharging circuit.

Device 500 can be constructed and arranged to deliver light, electromagnetic waves (e.g. microwaves), electrical currents (e.g. electrical currents for electrolysis), and/or electromagnetic fields, each of which, singly and/or in combination, can be delivered continuously or in a pulsed manner. Light emitted by device 500 can comprise one or more wavelengths of light delivered simultaneously and/or sequentially, such as multiple wavelengths delivered to provide different therapeutic effects to the patient.

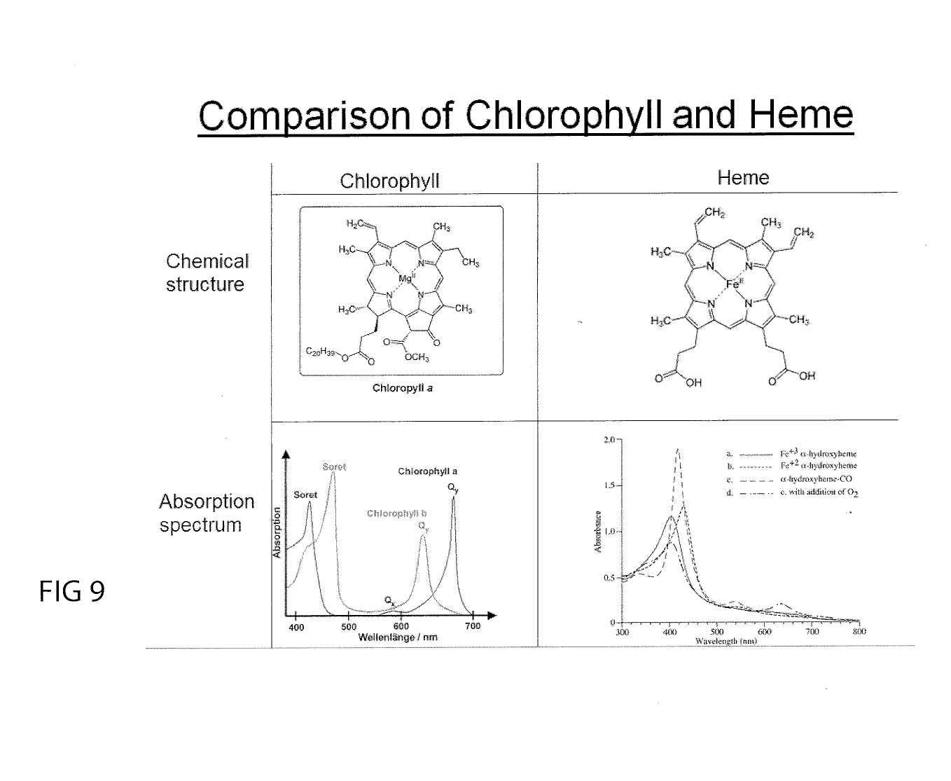

The molecule for oxygen transport is heme, a protoporphyrin ring molecule coupled to four different protein chains, which alter the chemical affinity of the heme. The heme molecule, which is one of the most constant chemical structures across different animal species, has also a high similarity with chlorophyll, the molecule responsible for photosynthesis in plants (see FIG. 9). Chlorophyll is activated by sunlight and acts as a chemical catalyst for photosynthesis. Apart from the central ion, Molybdan for chlorophyll and iron for heme, the two molecules have a very similar structure. As a consequence, heme can also absorb visible light (FIG. 9). The devices and methods of the present inventive concepts can be constructed and arranged to take advantage of the phototherapy of heme molecules. Heme is a molecule not only prevalent in hemoglobin, but also in various enzymes such as the nitric oxygen synthase (e.g. eNOS) as well at least 30 other heme enzymes (e.g. Cytochrom C, prostacyclin synthase, Methyltetrahydro protoberberin 14-monoxygenase, Leukotriene-monoxygenase and Hydroxylsamine oxidase). The devices and methods of the present inventive concepts can be constructed and arranged to deliver light to induce the catalytic function of one or more of these heme enzymes.

The effect of visible light on vascular function has been previously described, mainly by R. F. Furchgott, who was awarded Noble Prize Winner for his discovery of endothelium dependent vasodilation by nitric oxide produced by irradiation with visible light. Huang et al. (2012) described intravenous laser therapy being used to generate nitric oxide in mammals. Liu et al. (2008) described the effect of extracorporeal illumination generating increased levels of nitric oxides during phototherapy with newborns. Borisenko et al. (1997) described mechanisms including induction of nitric oxide synthase, a heme enzyme, or a dissociation of nitric oxide from NO-Hemoglobin. Kobayashi et al. (2000) observed an increase in peripheral blood flow due to extraocular direct irradiation of visible light in rats, which can have been effected by nitric oxide dependent mechanisms (e.g. and not by temperature). Peng et al. (2011) showed beneficial effects of phototherapy on atherosclerotic plaques in rabbits, which can have been effected by elevated levels of nitric oxide. Ortu et al. (1992) used intra-arterial phototherapy to prevent restenosis after balloon dilatation and stent implantation, which can have been a result of an antiproliferative effect preventing intima hyperplasia. Gurney et al. (1989) described an effect of light on cardiac calcium current, where intracellular calcium was increased by phototherapy, which could be used to increase cardiac contractility. Lawitschka et al. (2012) and other publications have reported a favorable effect of extracorporeal phototherapy in combination with photosensitizing agents for immunosuppressive therapy after organ transplantation, mostly after bone marrow transplantation with graft versus host disease.

Several cardiovascular diseases still lack an effective treatment with substantial improvement of prognosis. One of the most prominent is primary pulmonary hypertension, a disease in which elevated vascular resistance in the lungs often leads to right heart failure and premature death. Therapy using endothelin receptor antagonist has raised hope for clinicians and patients. A major pathophysiologic mechanism for pulmonary hypertension is the lack of nitric oxide in the pulmonary vascular bed, which as of yet has not been substantially improved with pharmacologic therapy. Evans et al. (2011) described pulmonary vasoconstriction being triggered by heme-oxygenase 2, which acts as a physiologic O₂ sensor leading to profound vasoconstriction at the pulmonary vascular bed during hypoxia.

The light or other energy delivered by energy delivery elements 555 of the present inventive concepts can be constructed and arranged to provide phototherapy and/or thermal therapy to treat one or more patient diseases or disorders. The light or other energy delivered by energy delivery elements 555 can be constructed and arranged to cause one or more physiologic effects.

In some embodiments, one or more energy delivery elements 555 are constructed and arranged to heat blood.

In some embodiments, one or more energy delivery elements 555 are constructed and arranged to alter the oxygen dissociation curve.

In some embodiments, one or more energy delivery elements 555 are constructed and arranged to cause vasodilation.

In some embodiments, one or more energy delivery elements 555 are constructed and arranged to increase nitric oxide, such as a local increase in nitric oxide.

In some embodiments, one or more energy delivery elements 555 are constructed and arranged to enhance nitric oxide release from the vascular endothelium.

In some embodiments, one or more energy delivery elements 555 are constructed and arranged to prolong local nitric oxide effects.

In some embodiments, one or more energy delivery elements 555 are constructed and arranged to cause an alteration in the function of erythrocytes.

In some embodiments, one or more energy delivery elements 555 are constructed and arranged to cause a modification in oxygen release from hemoglobin. The modification can be due to an interference with the heme-heme interaction. The modification can be caused by an electrostatic stabilization of deoxyhemoglobin the T-form. The modification can be caused by an increase in blood temperature due to heating of the blood by the energy delivery elements 555.

In some embodiments, one or more energy delivery elements 555 are constructed and arranged to cause a modification in the pH of blood, such as an increase and/or a decrease in blood pH.

In some embodiments, one or more energy delivery elements 555 are constructed and arranged to cause a modulation in the immune response of blood leucocytes. The modulation can be caused to prevent: organ rejection; graft versus host disease; and/or an autoimmune disease.

In some embodiments, one or more energy delivery elements 555 are constructed and arranged to cause a modulation of the coagulation and/or thrombocyte function. The modulation can be caused in order to increase coagulability and/or to inhibit blood coagulation.

In some embodiments, one or more energy delivery elements 555 are constructed and arranged to cause a modification in the function of heme catalyst enzymes in the blood. For example, the enzymes can include but are not limited to: catalase; endothelial nitric oxide synthase (ENOS); cytochrome; and/or myoglobin.

In some embodiments, one or more energy delivery elements 555 are constructed and arranged to cause a modification of hormonal action of one or more peptide and/or non-peptide hormone.

In some embodiments, one or more energy delivery elements 555 are constructed and arranged to cause a modification in the binding capacity of one or more antibodies.

In some embodiments, one or more energy delivery elements 555 are constructed and arranged to cause a decrease in blood glucose level. The glucose decrease can be caused by an electrochemical action that results due to the energy delivered by one or more energy delivery elements 555. Device 500 can include the delivery of one or more reagents configured to cause the glucose reduction. In some embodiments, no reagents are delivered by device 500.

In some embodiments, one or more energy delivery elements 555 are constructed and arranged to affect circulating tumor cells by selective heating, photocoagulation and/or photolysis.

In some embodiments, the device is constructed and arranged to causes electrolysis of blood, such as to prevent blood clotting proximate the device.

In some embodiments, device 500 is constructed and arranged to heat blood to a temperature of approximately 40° C. such as to facilitate hemoglobin oxygen unloading. Device 500 can deliver phototherapeutic light as well as energy configured to heat blood, such as phototherapeutic light plus infrared light in the absorption spectrum of oxy-Hb and/or radiofrequency energy configured to warm blood. The radiofrequency energy can comprise energy at a frequency of 200 kHz to 2000 Mhz, such as energy at a frequency between 500 MHz and 2000 MHz, or between 200 kHz and 1000 kHz, to facilitate the oxygen unloading by heating the blood.

Referring specifically to FIG. 7, a side sectional view of a device for delivering phototherapy is illustrated, consistent with the present inventive concepts. Device 500a can be constructed and arranged to deliver phototherapy and/or thermal therapy as has been described hereabove. Device 500a includes energy delivery elements 555a and 555b, collectively energy delivery elements 555. Energy delivery elements 555 can be constructed and arranged to deliver one or more forms of electromagnetic energy such as light energy selected from the group consisting of: visible light energy; infrared light energy; and combinations thereof. Device 500a and energy delivery elements 555 can be constructed and arranged to deliver electromagnetic energy into blood proximate a target organ such as an organ selected from the group consisting of: lung; heart; brain; kidney; liver; a transplanted organ; and combinations of these. The energy delivery elements 555 can be constructed and arranged to cause an effect selected from the group consisting of: improved perfusion; improved oxygen delivery by better oxygen unloading; improved metabolic function of heme-enzymes; improved immunosupression by irradiation of white blood cells; and combinations of these.

The treatment delivered by device 500a can be performed alone or in conjunction with one or more agents 660, such as an agent selected from the group consisting of: a nitric oxide donor; a photosensitizer; a photocatalyst; an enzyme substrate; and combinations of these. Agent 660 can comprise a photosensitizer and/or a photocatalyst. Agent 660 can comprise an agent configured to enhance the photodynamic effects of the phototherapy provided by one or more energy delivery elements 555. Agent 660 can comprise an agent selected from the group consisting of: metal; protein; carbohydrate; fatty acid; nucleic acid; synthetic medication; antibody; ionic solution; heme group; flavin group; aminolaevulin; phenol; polycarbon; fluorescent agent; plant derived agent such as chlorophyll; and combinations of these. Agent 660 can comprise a reagent, such as a reagent delivered by device 500a, such as to cause a reduction in blood glucose level. Agent 660 can comprise a photosensitizer or other agent that can be delivered from shaft 610 of device 500, such as via a pump element, not shown. Agent 660 can be stored in a reservoir within housing 620 or shaft 610. Alternatively or additionally, agent 660 can be injected intravenously, inhaled and/or administered as an oral pill.

Device 500a can be constructed and arranged for use in intensive care units, heart catheterization laboratories and/or other clinical care locations. The intravascular phototherapy for heme compounds delivered by device 500a can increase precapillary levels of nitric oxide and can provide vasodilation thereby, such as to improve perfusion in a target organ. Device 500a can be constructed and arranged to treat pulmonary hypertension. Device 500a can be constructed and arranged to deliver intravascular phototherapy to modify oxygen sensors and/or to cause a right shift of the hemoglobin dissociation curve of FIG. 8 as described hereabove. The shift can be accomplished by a photodynamic effect and/or by a thermal effect caused by the energy delivered by energy delivery elements 555 (e.g. infrared waves, microwaves, etc.) that results in increased blood temperature. Increasing the temperature by 1° C. can improve oxygen unloading by approximately 6% or more, analogous to the body's fever response. Similarly, an increase of 3° C. can result in an improvement in oxygen delivery of approximately 18% or more. By increasing precapillary O₂ saturation and nitric oxide level, pulmonary hypertension can be lowered and heart failure can be reduced.

In some embodiments, device 500a and energy delivery elements 555 are constructed and arranged to deliver energy to treat non-cardiac organs, such as the kidneys in renal failure; the liver in acute hepatic failure; the brain during and/or after a stroke; and/or a transplanted organ in acute rejection.

In some embodiments, one or more energy delivery elements 555 comprise a light emitting diode. In order to irradiate a target blood volume (e.g. a maximum blood volume), multiple light emitting diodes can be mounted along one or more shafts 610 and positioned in two or three dimensional orientations. The one or more shafts 610 can be constructed and arranged to be placed in an artery, vein, or other blood conducting conduit or chamber, including but not limited to: a pulmonary artery; a chamber of the heart; and/or the hepatic artery. In some embodiments, the light or other energy delivered by device 500a can be titrated to a clinical response. In some embodiments, the light or other energy delivered by device 500a can be pulsed (e.g. turned on and off), or simply turned off after a sufficient clinical improvement is achieved and/or pre-determined dose is delivered. In some embodiments, energy delivery elements 555 are positioned to deliver energy to a site specific (i.e. non-systemic) location, such as a volume of blood proximate a target organ (e.g. blood within an artery proximal to the target organ).

As described above, device 500a is configured for insertion into a natural body orifice or a blood vessel (e.g. via percutaneous access of the blood vessel). Device 500a includes shaft 610 comprising proximal end 611 and distal end 612, as well as outer surface 613. Proximal end 611 is attached to housing 620. Shaft 610 can be flexible or rigid, or it can contain both flexible and rigid portions. Shaft 610 can include one or more lumens, such as central lumen 615. Central lumen 615 is defined by inner surface 614 of shaft 610. In some embodiments, device 500a is configured for insertion over a guidewire, such as a guidewire inserted through central lumen 615, or a guidewire inserted in a distal sidecar, not shown but known to those of skill in the art to permit rapid exchange insertion of device 500a over a guidewire. A wall 617 of shaft 610 is positioned between inner surface 614 and outer surface 613. In some embodiments, shaft 610 includes multiple lumens, such as when device 500a comprises a multi-lumen catheter for insertion into a blood vessel or other body location. Shaft 610 can include a generally smooth outer surface 613, and can include a relatively uniform outer profile (at least in the distal portion), such as an outer profile between 2 mm and 9 mm in diameter.

Shaft 610 can include one or more energy delivery elements, such as one or more of energy delivery elements 555a and 555b as shown, configured to deliver light or other energy such as to provide phototherapy and/or thermal therapy as described hereabove. The energy delivery elements of the present inventive concepts can comprise an element that both produces and delivers light (e.g. a light emitting diode or other light generating element) and/or these elements can simply deliver light (e.g. a segment of exposed optical fiber or other optical element attached to a light source as described herein). The energy delivery elements of the present inventive concepts can include one or more optical components, such as one or more of the optical elements described herein. An optical element can be constructed and arranged to couple light into an energy delivery element 555, such as optical element 657 described herebelow. Alternatively or additionally, an optical element can be configured to distribute light from an energy delivery element, such as a lens, prism or other optical element configured to distribute light in one or more desired patterns. Optical elements of the present inventive concepts can comprise an optical element selected from the group consisting of: lens; ball lens; prism; diffractor; filter; mirror; optical fiber; and combinations thereof.

The cross section of FIG. 7 illustrates multiple energy delivery elements 555 positioned within an axial segment of shaft 610, such as to deliver light or other energy radially out from and/or radially into shaft 610 (e.g. into a lumen 615 of shaft 610), as shown by the arrows emanating from each energy delivery element 555a. One or more energy delivery elements 555b (one shown in FIG. 1) can be positioned within a distal portion of shaft 610, such as to transmit light radially out from shaft 610, as shown by the arrows emanating from energy delivery element 555b. In some embodiments, energy delivery element 555b is constructed and arranged to be positioned within a chamber of the heart, or other site specific location. Energy delivery elements 555a and 555b are operably connected to one or more sources of light, such as is described herebelow. In some embodiments, device 500a includes one or more energy delivery elements 555a (e.g. positioned along the majority of the length of shaft 610) and no energy delivery element 555b. In other embodiments, device 500a includes energy delivery element 555b (e.g. positioned in a distal portion of shaft 610) and no energy delivery elements 555a.

Device 500a can include one or more optical fibers, such as optical fibers 651a positioned within wall 617 of shaft 610. One or more fibers 651a can each be embedded in wall 617 of shaft 610. Alternatively or additionally, one or more fibers 651a can be insertable into (e.g. slidingly received by) a lumen, such as lumens 618 positioned within wall 617 (lumens 618 are omitted from FIG. 7 for illustrative clarity but shown in FIG. 7A and described herebelow). Alternatively or additionally, one or more fibers 651a can be positioned within central lumen 615. In some embodiments, central lumen 615 comprises multiple separate lumens and one or more optical fibers 651a can be positioned within and/or insertable into one or more of the multiple lumens of central lumen 615. Fibers 651a include an axial portion covered by an opaque covering, cladding 652a, such as the covered proximal portions of each fiber 651a shown in FIG. 7. Each energy delivery element 555a comprises an axial portion of a fiber 651a that is not surrounded by an opaque material (e.g. the uncovered distal segments of each fiber 651a with length D1 shown in FIG. 7). Light introduced into the proximal end of each fiber 651a is conducted along the cladded optical fiber with minimal losses of light, as is known to those of skill in the art. However when reaching the uncladded portion defined by energy delivery element 555a, the conducted light or other energy emanates radially out from the associated energy delivery element 555a (e.g. a portion of fiber 651 that is not surrounded by cladding and can be modified to enhance the distribution of light or other energy), as shown by the arrows emanating from energy delivery element 555a shown in FIG. 7. During use, energy delivery element 555a can be positioned in an area of the patient to be treated (e.g. phototherapy or thermal therapy delivered), such as within a segment of a blood vessel.

Each energy delivery element 555a can comprise a modified surface of fiber 651, such as a roughened surface and/or a surface otherwise modified to enhance the distribution of light or other energy. In some embodiments, one or more energy delivery elements 555a can comprise a segment of optical fiber 651 that has received a surface treatment selected from the group consisting of: etching; cutting; covering with roughened material such as silicon; and combinations thereof. In some embodiments, energy delivery element 555a comprises a segment of optical fiber 651 including at least a portion that is surrounded by a transparent covering. The transparent covering can include one or more reflective particles constructed and arranged to diverge radiated light, such as one or more particles selected from the group consisting of: alumina particles; silica particles; titania particles; titanium oxide particles; and combinations of these.

Device 500a can include one or more optical fibers 651b extending from housing 620 to a distal portion of shaft 610. One or more optical fibers 651b can be positioned within lumen 615 and/or within wall 617 of shaft 610. Energy delivery element 555b can comprise one or more optical elements (as described hereabove) positioned on the distal end of the one or more optical fibers 651b, such as optical element 657 shown, which couples light from fiber 651b into energy delivery element 555b. In some embodiments, energy delivery element 555b comprises a ball lens that is attached to the distal end of a fiber 651b or a ball lens that is formed from the distal end of fiber 651b. A ball lens can be created by melting the end of fiber 651b with applied heat. Alternatively, energy delivery element 555b can comprise an uncovered distal portion (e.g. not covered by cladding 652b) as described hereabove in reference to energy delivery element 555a. In some embodiments, optical fiber 651b and energy delivery element 555 are configured to translate through lumen 615, such as to exit the distal end 612 of shaft 610. Alternatively, energy delivery element 555 can exit a side hole in an exterior portion of

Optical fibers 651a and/or 651b can comprise one or more materials configured to propagate the light for delivering phototherapy and/or thermal therapy. Alternatively, optical fibers 651a and/or 651b can comprise one or more wires or other conduits configured to conduct non-light energy. In some embodiments, fibers 651a and/or 651b comprise a material selected from the group consisting of: glass; plastic; polymethylmethacrylate (PMMA); one or more polymers (e.g. one or more polymers configured as a microstructured polymer optical fiber); photonic crystal; polycarbonate; polystyrene; and combinations of these. Fibers 651a and/or 651b can comprise flexible fibers which allow movement within the patient without breaking or other issues.

The proximal end of each fiber 651a is attached and/or attachable to a light and/or other energy source, such as energy source 650a shown. The proximal end of each fiber 651b is attached and/or attachable to a light and/or other energy source, such as energy source 650b shown. Device 500a further includes a battery, capacitor or other power source, such as power supply 653 shown positioned in housing 620 and operably connected to energy sources 650a and 650b. In some embodiments, energy sources 650a and 650b (collectively energy source 650) comprise a single energy source 650 attached to one or more power supplies 653. Power supply 653 can comprise a replaceable or rechargeable power source, such as a rechargeable power source included when an implanted housing 620 surrounds power supply 653, such as implantable housing 820 and power supply 853 of FIG. 11 described herebelow. In some embodiments, one or more energy sources 650 comprises a non-light energy source, such as when optical fibers 651a and/or 651b comprise a wire or other conduit configured to transmit non-light energy to one or more energy delivery elements 555, such as when one or more energy delivery elements 555 comprise an LED or other electrically powered light source, or when one or more energy delivery elements 555 comprise a non-light energy delivery element.

Energy source 650 can be positioned within housing 620 (as shown in FIG. 7), on and/or within shaft 610, or at an external location (as shown in FIG. 2). Energy source 650 can be constructed and arranged to provide light at a single wavelength. Alternatively or additionally, energy source 650 can be constructed and arranged to provide light at multiple wavelengths, simultaneously or pulsed. The light can be provided to one or more energy delivery elements 555, such as via one or more optical fibers 651 or other light or other energy carrying conduit. Energy source 650 can comprise an LED, such as an organic LED, described hereabove. In some embodiments, an energy delivery element 555 and energy source 650 are positioned proximate each other, are attached to each other, and/or comprise the same component. In some embodiments, energy delivery element 555b comprises energy source 650 which can comprise an LED, lamp, laser, or other light source positioned within a distal portion of shaft 610.

Energy source 650 can comprise a laser, such as a diode laser or other laser configured to provide one or more wavelengths of light. Energy source 650 can be constructed and arranged to deliver pulsed light, such as light delivered at a duty cycle between 0.1% and 50%. Energy source 650 can provide light at a power of less than 100 Watts, or less than 50 Watts. In some embodiments, energy source 650 provides light at less than or equal to 100 Watts that is pulse-width modulated to deliver light at less than 50 Watts rms, or less than 20 Watts rms, to one or more energy delivery elements 555. In some embodiments, energy source 650 provides light at a power of at least 0.1 Watts, such as at a power between 2 Watts and 10 Watts, or such as between 0.3 Watts and 0.6 Watts. Energy source 650 can provide one or more energy wavelengths. In some embodiments, light source 150 is configured to deliver light at a power of between 1.0 mW and 100 mW. In some embodiments, the light delivered by one or more light delivery elements 155 is delivered at a power between 1.0 mW and 100 mW. In some embodiments, light delivery element 155 delivers light to tissue at a power density between 1.0 mW/cm² and 10.0 mW/cm².

Energy source 650 can be constructed and arranged to provide such as one or more wavelengths between 410 nm and 580 nm, such as between 410 nm and 420 nm, such as between 540 nm and 550 nm, such as between 570 nm and 580 nm. Energy source 650 can be constructed and arranged to provide visible light and/or ultraviolet light. Energy source 650 can be constructed and arranged to deliver light at varying wavelengths, such as light provided at continuously varying wavelengths or light alternative between at least a first wavelength and a second wavelength. Energy source 650 can be constructed and arranged to provide light at multiple wavelengths simultaneously. In some embodiments, multiple wavelengths are provided by energy source 650 to enhance the phototherapeutic effects of device 500a. As described above in reference to FIG. 1, one or more wavelengths delivered by energy source 650 are based on the absorption spectra of one or more microorganisms to be treated. For example, a wavelength of approximately 405 nm can be delivered to treat E. Coli; a wavelength of 470 nm can be delivered to treat staphylococcus; and/or a wavelength of 670 nm can be delivered to treat oral candida. Alternatively or additionally, one or more wavelength may be delivered to cause a vascular effect (e.g. to mediate nitric oxide), such as one or more wavelengths approximating 350 nm and/or 430 nm.

Energy source 650 and/or one or more energy delivery elements 555 can be constructed and arranged to provide light to an area (e.g. an area or volume of blood or an area of tissue) at a power density less than 50 mW/cm², such as at a power density less than 25 mW/cm², or at a power density approximately equal to or less than 10 mW/cm². In some embodiments, energy source 650 and/or one or more energy delivery elements 555 are constructed and arranged to deliver light or other energy at approximately 1.0 mW/cm² to 10 mW/cm². In some embodiments, energy source 650 and/or one or more energy delivery elements 555 are constructed and arranged to deliver between OA mW/cm² and 100 mW/cm², between 2 mW/cm² and 10 mW/cm², between 2 mW/cm² and 8 mW/cm², or between 50 mW/cm² and 300 mW/cm². In some embodiments, energy source 650 and/or one or more energy delivery elements 555 are constructed and arranged to deliver light or other energy at a level to prevent tissue dehydration or other adverse tissue effect, such as pulse-width modulated light delivery configured to prevent tissue dehydration or cell damage.

Heating of the surrounding blood or other tissue can be monitored by one or more sensors of device 500a, such as a sensor 619 when sensor 619 comprises a thermocouple, thermister or other temperature sensor. Sensor 619 can be positioned on shaft 610, such as in a distal portion of shaft 610 as shown, or at another device 500a location. Sensor 619 can be operably attached to, or operably attachable to, sensor measurement assembly 200, such as via one or more conduits, not shown but typically including one or more wires or optical fibers. In some embodiments, sensor 619 comprises a thermocouple or thermistor attached to assembly 200 with one or more wires such that sensor measurement assembly 200 can produce a temperature measurement used to regulate the light and/or other energy provided by energy source 650. In some embodiments, sensor 619 can comprise one or more optical fibers (e.g. one or more optical fibers 651), which collect infrared light (e.g. from their distal end). In these embodiments, sensor measurement assembly 200 comprises an infrared sensor configured to correlate the received light to a temperature. The temperature determined by assembly 200 (e.g. via thermocouple signal, thermisters signal, infrared signal or otherwise), can be used to regulate the light delivered by energy source 650 in a closed loop fashion, such as to prevent damage to tissue and/or to optimize the phototherapy and/or thermal therapy being delivered by device 500a. Measurement assembly 200 can include a microprocessor and/or other electronic circuitry to receive one or more signals from sensor 619 to determine a measured temperature. In some embodiments, sensor 619 comprises an oxygen sensor, such as an oxygen saturation sensor used to titrate the effect of oxygen unloading. In these embodiments, sensor 619 can comprise a sensor constructed and arranged to determine the difference in absorption spectrum red (650 nm to 750 nm) for dexoy-Hb and infrared 860-1000 nm for oxy-Hb.

Energy delivery elements 555a can be constructed and arranged to deliver light radially out from all or a portion of outer surface 613 of shaft 610, such as a majority portion and/or distal portion of the outer surface 613. Alternatively or additionally, energy delivery elements 555a can be constructed and arranged to deliver light radially in from all or a portion of inner surface 614 of shaft 610, such as a majority portion and/or distal portion of the inner surface 614. In some embodiments, an energy delivery element 555 is positioned to direct light toward a skin surface such as a skin surface surrounding a natural opening such as the skin surface surrounding the urethral orifice, or to a skin surface surrounding a skin incision and/or skin penetration site. In some embodiments, an energy delivery element 555 comprises one or more optical components (e.g. a lens) configured to distribute light to a skin surface, not shown but such as is described in reference to FIG. 2 hereabove.

The energy delivery elements 555 of the present inventive concepts can comprise one or more optical elements. In some embodiments, an energy delivery element 555 can comprise an optical element selected from the group consisting of: optical fiber; lens; ball lens; prism; diffractor; filter; mirror; and combinations of these. An optical element can be positioned at the end of an optical fiber, such as an energy delivery element 555b comprising a ball lens positioned at the end of fiber 651b, as described hereabove. In some embodiments, one or more energy delivery elements 555 comprise light scattering material, not shown but such as is described in detail in reference to FIG. 2 hereabove. The light scattering material can comprise a material distributed relatively evenly throughout a silicone or other polymer material, such as a light scattering material selected from the group consisting of: alumina particles; silica particles; titania particles; titanium oxide particles; and combinations of these. In some embodiments, device 500a can be constructed and arranged to rotate one or more energy delivery elements 555 (or a portion of an energy delivery element 555), such as to distribute light and/or other energy over a larger area, rotating assembly not shown but described in detail in reference to FIG. 6 hereabove. One or more energy delivery elements 555 can be constructed and arranged to deliver light to a majority of a cavity, such as a lens, prism, diffractor and/or mirror configured to direct light toward the majority of the surface of a heart chamber or other organ surface and/or the majority of a segment of a blood vessel wall.

The energy delivery elements 555, shaft 610 and/or another component of device 500a can comprise a fluorescent material, such as a material constructed and arranged to increase light dispersion. In some embodiments, the fluorescent material can be positioned at least on the outer surface 613 and/or inner surface 614 of shaft 610, such as a fluorescent coating placed upon surfaces 613 and/or 614 of shaft 610. Other coatings can be included on one or more components of device 500a, such as a photosensitizer constructed and arranged to be activated by light (e.g. light delivered by an energy delivery element 555) to cause a phototherapeutic and/or thermal therapeutic effect. Shaft 610 can include one or more portions that are transparent or at least translucent (hereinafter “translucent”) to one or more wavelengths of light transmitted by an energy delivery element 555. Shaft 610 can include a translucent portion surrounding an energy delivery element 555a comprising an unclad portion of an optical fiber 651a.

In some embodiments, device 500a can include an anchoring element or an expandable element, such as balloon, such as balloon 125 of FIG. 1 described hereabove.

Housing 620 can be configured as a handle for a user, such as a clinician or patient, to hold while inserting, rotating and/or otherwise using device 500a. Housing 620 can be implantable or include an implantable portion, such as is described in reference to FIG. 11 herebelow. Housing 620 can comprise a first housing and a second housing separated from the first housing. Housing 620 can surround one or more components including but not limited to: power supplies such as batteries; agent reservoirs such as pharmaceutical agent reservoirs; pumping mechanisms; energy delivery circuitry such as cardiac pacing or defibrillating circuitry; electronic processing circuitry; electronic memory circuitry; and combinations of these.

Device 500a can be constructed and arranged to perform numerous functions in addition to the delivery of phototherapy and/or thermal therapy. In some embodiments, device 500a comprises a device selected from the group consisting of: vascular access device; central venous catheter; peripherally inserted central catheter such as a peripherally inserted venous catheter; drug or other agent delivery pump; implanted device; implanted drug or other agent delivery pump; pacemaker; drive shaft assembly for a cardiac assist device; inflow and/or outflow cannula for a cardiac assist device; artificial heart; and combinations of these.

In some embodiments, shaft 610 is constructed and arranged for insertion through an incision of the skin, such as when shaft 610 is further inserted into a blood vessel, organ, and/or a subcutaneous tissue tunnel. In some embodiments, distal end 612 of shaft 610 is inserted into a blood vessel such that blood can be removed via lumen 615 and/or one or more agents can be delivered to an internal body location (e.g. into the cardiovascular system of the patient) via lumen 615. In these embodiments, housing 620 can include a luer or other attachment element configured to add or remove fluids via lumen 615, such as when device 500a comprises a central venous catheter or peripherally inserted central catheter. In some embodiments, shaft 610 is inserted through the skin and into a blood vessel supplying blood to an organ such as the kidney, liver or heart. Alternatively, device 500a can comprise an external (i.e. non-implanted) drug delivery pump, such as a skin-attached or other external drug delivery device in which shaft 610 comprises a transcutaneous conduit (e.g. a rigid needle or flexible catheter passing through the skin surface into a blood vessel) for agent delivery. In these embodiments, housing 620 can include a pumping mechanism, not shown but described in reference to FIGS. 4 and 4A herebelow. In some embodiments, the agent delivered comprises an agent selected from the group consisting of: insulin; a chemotherapeutic agent; a nutritional material; a pain control agent such as morphine; and combinations thereof.

The energy delivery elements 555 of the present inventive concepts can be constructed and arranged to deliver a minimum amount of light and/or other energy to blood and/or other tissue. Alternatively or additionally, the energy delivery elements 555 of the present inventive concepts can be constructed and arranged to deliver a maximum of light and/or other energy to blood and/or other tissue.

In some embodiments, shaft 610 and housing 620 are constructed and arranged for implantation into the patient, such that energy delivery elements 555a and/or 555b can deliver light or other energy to blood surrounding at least a portion of shaft 610. Shaft 610 can be constructed and arranged for insertion through a subcutaneous tissue tunnel or through another internal body location. Distal end 612 can be constructed and arranged for insertion into a blood vessel, heart chamber, or other internal body location. Device 500a can comprise an implanted portion configured to deliver a pharmaceutical agent or other agent systemically and/or locally to an internal location of the patient, such as to deliver an agent systemically or locally within the patient. The delivered agent can be configured to improve the phototherapeutic effect of light delivered by one or more energy delivery elements 555. In these embodiments, an implanted housing 620 can surround a drug reservoir and pumping means which deliver an agent to lumen 615 of shaft 610, drug reservoir and pumping means not shown but described in detail in reference to FIGS. 5 and 5A hereabove.

In some embodiments, device 500a further comprises a light enhancing material, agent 660, such as a photosensitizer and/or a photocatalyst. Agent 660 can be included in one or more components of device 500a, such as a light enhancing coating applied to all or a portion of shaft 610. The coating can be configured to dissolve or otherwise migrate from shaft 610. Agent 660 can be delivered by one or more components of device 500a, such as from one or more openings in shaft 610 (e.g. lumen 615 at distal end 612) or from an outlet in another component of shaft 610. In some embodiments, agent 660 is constructed and arranged to enhance the photodynamic effect of device 500a, such as when agent 660 is a material selected from the group consisting of: metal; protein; carbohydrate; fatty acid; a nucleic acid; a synthetic medication; antibody; ionic solution; heme group; flavin group; aminolaevulin; phenol; a polycarbon; and combinations of these. In some embodiments, agent 660 comprises a material selected from the group consisting of: toluidine blue O; methylene blue; and combinations thereof. In some embodiments, the light enhancing material is constructed and arranged to be activated by light delivered by device 500a, such as light delivered by one or more energy delivery elements 555.

One or more components of device 500a can be provided sterile, such as a sterile shaft 610. One or more components of device 500a can be reusable and/or re-sterilizable. In some embodiments, shaft 610 is used in a single use on a single patient, while energy source 650 and/or one or more energy delivery elements 555 (e.g. fibers 651) are reused in multiple uses with one or more patients (e.g. when energy source 650 is operably attachable to fiber 651 and/or when fiber 651 is insertable into shaft 610).

In some embodiments, in addition to one or more energy delivery elements 555, device 500a includes one or more additional components constructed and arranged to further provide phototherapy and/or thermal therapy, such as functional element 695 shown in FIG. 7. Functional element 695 can be constructed and arranged to improve upon the phototherapy and/or thermal therapy than that which is achieved with the energy delivered by the one or more energy delivery elements 555 alone. Functional element 695 is connected to conduit 696, which can comprise one or more wires, optical fibers, or other energy carrying conduits. Conduit 696 travels proximally through shaft 610, and connects to a supply of energy, such as energy delivery unit 697. Energy delivery unit 697 can be contained within housing 620 (as shown), or at a location external to housing 620. Energy delivery unit 697 can be attached to power supply 653 or another supply of power via conduit 696. Conduit 696 can comprise at least two wires, such as two wires constructed and arranged to provide power to functional element 695 to create an electromagnetic field and/or an electric current (e.g. an electromagnetic field and/or electrical field provided to tissue and/or body fluid).

In some embodiments, functional element 695 comprises an element configured to deliver an electromagnetic field. Functional element 695 can produce one or more of: a dynamic electromagnetic field; a static electromagnetic field; a dynamic magnetic field; or a static magnetic field; a dynamic electrical field; or a static electrical field. Functional element 695 can produce a magnetic field with a field strength between 1 milliTesla (mT) and 500 mT. Functional element 695 can produce a magnetic field constructed and arranged to prevent adversely effecting one or more muscles and/or nerves. Alternatively or additionally, functional element 695 can comprise an element configured to deliver an electric current and/or electric potential. The delivered electric current and/or electric potential can be configured to cause electrolysis. Alternatively or additionally, functional element 695 can comprise an ultrasound transducer, and the produced ultrasound waves can be configured to enhance the phototherapy and/or thermal therapy provide by device 500a. Energy delivery unit 697 can provide electrical power, ultrasound signals, or other energy used to energize functional element 695.

Functional element 695 can be positioned on and/or in shaft 610, such as on and/or in a distal portion of shaft 610 or at distal end 612 of shaft 610. In some embodiments, functional element 695 comprises one or more elements positioned on and/or within a balloon (not shown but such as balloon 125 of FIG. 1), such that functional element 695 traverses radially from shaft 610 as the balloon is expanded. In some embodiments, functional element 695 is positioned along a majority of the length of shaft 610, or along a majority of the length of a portion of shaft 610 positioned in the patient's blood. In some embodiments, functional element 695 delivers a phototherapeutic or thermal therapeutic effect (e.g. via delivery of an electric current and/or electric potential, electromagnetic field and/or ultrasound waves) to similar locations of tissue and/or body fluid to that receiving energy from the one or more energy delivery elements 555. In some embodiments, functional element 695 comprises multiple functional elements, such as multiple functional elements selected from the group consisting of: one or more electromagnetic field generating elements; one or more electric current and/or electric potential delivering elements; one or more ultrasound transducers; and combinations thereof. The multiple functional elements 695 can be, singly or in combination, constructed and arranged to enhance the phototherapeutic effect and/or thermal therapeutic effect of device 500a.

One or more functional elements 695 comprise one or more permanent magnets. In some embodiments, functional element 695 comprises multiple permanent magnets, such as multiple permanent magnets positioned on or in shaft 610. The multiple permanent magnets can be dispersed relatively uniformly along one or more portions of the length of shaft 610, such as along a full or partial circumferential portion of shaft 610, along a majority of the length of shaft 610 and/or along a majority of the length of shaft 610 which is positioned in the blood of the patient. In some embodiments, functional element 695 comprises multiple permanent magnets that are dispersed non-uniformly, such as along a distal, mid or proximal portion of shaft 610.

Referring now to FIG. 7A, a cross sectional view of device 500a of FIG. 7 at line A-A is illustrated, consistent with the present inventive concepts. Shaft 610 includes wall 617 and central lumen 615. Positioned within wall 617 are an array of multiple optical fibers 651 (sixteen fibers 651 shown), such as optical fibers 651a or 651b of FIG. 1. Surrounding optical fibers 651 at section A-A is cladding 652. Cladding 652 is not included in the more distal portion of fibers 651 as described above. Wall 617 can include multiple lumens 618 configured to slidingly receive optical fibers 651. Also positioned within wall 617 is conduit 696 described hereabove.

Referring specifically to FIG. 10, a schematic view of an energy delivery device comprising multiple shafts and multiple energy delivery elements is illustrated, consistent with the present inventive concepts. Device 500b can be constructed and arranged to deliver phototherapy and/or thermal therapy as has been described hereabove. Device 500b comprises shafts 710a-d (collectively shafts 710) with energy delivery elements 555a-d, respectively. Each energy delivery element 555a-d can comprise one or more energy delivery elements, as shown, such as multiple energy delivery elements positioned along the majority of the length of each shaft 710a-d, respectively. Energy delivery elements 555a-d (collectively energy delivery elements 555) can be constructed and arranged to deliver one or more various forms of energy, such as has been described hereabove. Energy delivery elements 555 are operably attached to one or more energy carrying conduits, not shown but such as one or more wires or optical fibers that deliver energy to energy delivery elements 555 from energy source 750. In some embodiments, energy is delivered by energy source 750 to distribution element 761, from which energy is individually distributed to the energy delivery elements 555.

In some embodiments, energy delivery elements 555 comprise light energy delivery elements. In these embodiments, energy delivery elements 555 can comprise a light source such as an LED that is attached to one or more wires that travel proximally connecting to distribution element 761 and/or energy source 750. Alternatively, energy delivery elements 555 can comprise an optical fiber, lens or other optical component as has been described hereabove to deliver light energy. The optical component can be attached to one or more optical fibers which optically attach to distribution element 761 and/or energy source 750 (e.g. a laser or other light energy source). In some embodiments, shafts 710 comprise an optical fiber that comprises one or more energy delivery elements 555 (e.g. one or more unclad portions of an optical fiber as described hereabove).

On the proximal end of device 500b is a handle, housing 720, which includes control 728. Control 728 comprises a user interface component that can be configured to initiate energy delivery by the multiple energy delivery elements 555 and/or modify the energy delivery provided.

Shafts 710a-d can be configured to be inserted individually or in multiple into one or more blood vessels of the patient, such as one or more pulmonary arteries of the patient. Shafts 710a-d can be configured to deliver phototherapy and/or thermal therapy to the blood passing through the one or more pulmonary arteries as has been described hereabove.

Referring specifically to FIG. 11, a schematic view of an implantable energy delivery device comprising at least one energy delivery element is illustrated, consistent with the present inventive concepts. Device 500c can be constructed and arranged to deliver phototherapy and/or thermal therapy as has been described hereabove. Device 500c includes an implantable housing 820 surrounding a power supply 853. In some embodiments, power supply 853 is a rechargeable battery, such as when device 500c is configured to allow recharging of power supply 853 via a transcutaneous transfer of energy selected from the group consisting of: inductive coupling of electromagnetic waves; transmission of microwaves; transmission of ultrasound waves; and combinations of these. In some embodiments, device 500c comprises an expandable scaffold 861 configured in a stent-like configuration and inserted into blood vessel BV as shown. Expandable scaffold 861 includes multiple energy delivery elements 555 configured to deliver light or other electromagnetic energy to blood passing within expandable scaffold 861. Alternatively, expandable scaffold 861 can be positioned on the outside of blood vessel BV, and energy delivery elements 555 can be configured to deliver light or other energy through the walls of blood vessel BV to be received by the blood passing therein. Energy delivery elements 555 are connected to energy source 850 via one or more cables 851, such as one or more fiber optic cables. In some embodiments, energy source 850 transmits light energy, ultrasound energy and/or mechanical energy over cable 851 to energy delivery elements 555. In some embodiments, energy source 850 comprises a light energy source, such as a light energy source delivering light of one or more wavelengths, such as one or more wavelengths between 250 nm and 730 nm, as has been described hereabove. For heating purpose, one or more wavelengths in the infrared and/or microwave spectrum can be applied (e.g. one or more wavelengths between 730 nm and 10 cm). Device 100 can be constructed and arranged to deliver one or more wavelengths of electromagnetic energy as well as modify the blood's pH (e.g. via electrolysis), such as to improve or at least alter the absorption of hemoglobin by altering the pH of hemoglobin.

Device 500c includes external controller 891 which includes user interface 898 and electronics module 899a. Electronics module 899a comprises a wireless transmitter and one or more other electronics components for providing a user interface and controlling one or more implanted components of device 500c. Implanted housing 820 surrounds electronics module 899b which comprises a wireless receiver which receives communications from external controller 891. In some embodiments, electronics module 899a and electronics module 899b are each configured as transceivers to allow two way communication between the implanted portion of device 500c and external controller 891. Controller 891 can be configured to allow adjustment of energy delivered by energy source 850 to one or more energy delivery elements 555 as has been described hereabove.

In some embodiments, electronics module 899a and electronics module 899b are constructed and arranged for wireless transfer of power from controller 891 to power supply 853, such as power transferred through inductive coupling and/or transcutaneous delivery of light.

Referring now to FIG. 12, a side sectional view of a device with a light delivery element positioned within a balloon is illustrated, consistent with the present inventive concepts. Device 100 includes shaft 110 comprising proximal end 111 and distal end 112, as well as outer surface 113. Proximal end 111 is attached to housing 120. Shaft 110 can be flexible or rigid, or it can contain both flexible and rigid portions. Shaft 110 can include one or more lumens, such as central lumen 115. Central lumen 115 is defined by inner surface 114 of shaft 110 and is operably attachable at its proximal end to an evacuation device such as a urine bag or other fluid evacuation assembly. A wall 117 of shaft 110 is positioned between inner surface 114 and outer surface 113. In some embodiments, shaft 110 includes multiple lumens, such as when device 100 comprises a multi-lumen catheter for insertion into a blood vessel or other body location. Shaft 110 can include a generally smooth outer surface 113, and can include a relatively uniform outer profile (at least in the distal portion) between 3 mm and 9 mm in diameter.

Shaft 110 can include an anchoring and/or expanding element, such as balloon 125 positioned on a distal portion of shaft 110. An anchor, such as balloon 125 or other expandable element can be used to anchor shaft 110 at an internal body location such as the bladder. In addition to anchoring shaft 110, inflation of balloon 125 can be configured to provide a function selected from the group consisting of: cooling one or more light delivery elements 155 as described herebelow; position one or more light delivery elements 155 at a minimum or target distance from tissue receiving light from the one or more light delivery elements 155. Balloon 125 is typically in a radially compacted (e.g. deflated) state during insertion shaft 110 into a body lumen such as the urethra. In its deflated state, balloon 125 provides a relatively smooth outer surface preventing any trauma to the patient from light delivery element 155 and/or bundle 154.

Light delivery elements 155 can comprise one or more light delivery elements that are positioned within balloon 125 such as to transmit light radially out from balloon 125, as shown by the arrows emanating from light balloon 125. Alternatively or additionally, light delivery elements 155 can comprise one or more light delivery elements positioned on, in or within shaft 110. In some embodiments, light delivery element 155 comprises a circumferential array of LEDs or other light delivery elements. In some embodiments, light delivery element 155 comprises an LED and a lens configured to near spherically distribute light (e.g. through the majority of the surface of balloon 125). During use, light delivery element 155 can be positioned in an area of the patient to be treated (e.g. infection reduced or prevented and/or therapeutic light delivered) such as within a segment of the urethra. Light delivery elements 155 are operably connected to the distal end of one or more energy carrying conduits, bundle 154, such as a flexible bundle of one or more optical fibers and/or electrical wires. Bundle 154 can comprise one or more optical fibers including a surrounding cladding layer (as described hereabove) and/or one or more electrically conductive wires surrounded by an insulating layer.

Bundle 154 can comprise one or more energy carrying conduits positioned within a lumen of shaft 110, within the wall of shaft 110, and/or on the outer surface of shaft 110. Bundle 154 can be operably connected on its proximal end to a source of power, power supply 153 such as a battery and/or capacitor electrically connected to light delivery element 155 via bundle 154. Power supply 153 can be positioned outside of shaft 110 and/or housing 120 to avoid applying a load to shaft 110, such as to prevent undesired forces on shaft 110 that may lead to patient discomfort. In these embodiments, power supply 153 may be attachable to a piece of patient's clothing (e.g. a belt) and/or furniture of the patient (e.g. a bed or a chair). In some embodiments, power supply 153 is positioned within housing 120. Power supply 153 can comprise a replaceable or rechargeable power supply, such as a replaceable battery or a rechargeable energy source, respectively. Power supply 153 can be attached to one or more switches or other controls, not shown but configured to allow an operator to selectively apply energy to light delivery element 155.

Light delivery element 155 can be configured to deliver light such as to prevent and/or reduce infection. Alternatively or additionally, device 100 and/or light delivery element 155 can be constructed and arranged to cause a physiologic effect selected from the group consisting of: blood temperature increase; vasodilation; increase in local nitric oxide; enhance nitric oxide release from the vascular endothelium; prolong local nitric oxide effects; alteration in the function of erythrocytes; modification in oxygen release from hemoglobin; modification in pH of blood; modulation in the immune response of blood leucocytes; modulation of the coagulation and/or thrombocyte function; modification in the function of heme catalyst enzymes in the blood; modification in hormonal action of a peptide and/or non-peptide hormone; modification in the binding capacity of one or more antibodies; decrease in blood glucose level; affect circulating tumor cells by selective heating, photocoagulation and/or photolysis; and combinations of these. In some embodiments, one or more light delivery elements 155 are constructed and arranged to deliver phototherapy, such as is described hereabove in reference to FIGS. 7-11.

The light delivery elements of the present inventive concepts can comprise an element that both produces and delivers light (e.g. a light emitting diode or other light generating element) and/or these elements can simply deliver the light (e.g. a segment of exposed optical fiber or other optical element attached to a light source as described herein). The light delivery elements of the present inventive concepts can include one or more optical components, such as the optical elements 157 described hereabove in reference to FIG. 1. An optical element can be constructed and arranged to couple light into a light delivery element, such as a lens or other optical element positioned to couple light from bundle 154 into a light delivery element 155. Alternatively or additionally, an optical element can be configured to distribute light from a light delivery element, such as a lens, prism or other optical element configured to distribute light in one or more desired patterns. Optical elements of the present inventive concepts can comprise an optical element selected from the group consisting of: lens; ball lens; prism; diffractor; filter; mirror; optical fiber; and combinations of these.

In some embodiments, light delivery element 155 comprises a light source constructed and arranged to deliver light through the walls of balloon 125 via power delivered by one or more electrical wires of bundle 154. Light delivery element 155 can comprise a light source selected from the group consisting of: LED; lamp; laser; and combinations thereof. Light delivery element 155 can comprise one or more organic LEDs. Light delivery element 155 can comprise a ball lens or other lens configured to deliver light in any or all directions from balloon 125.

Light delivery element 155 can comprise one or more LEDs with a dimension less than or equal to 1 mm. One or more light delivery elements 155 can be mounted to balloon 125 and/or shaft 110. One or more light delivery elements 155 can be constructed and arranged to deliver blue light. One or more light delivery elements 155 can be constructed and arranged to deliver multiple wavelength of light.

Light delivery element 155 can comprise a diode or other light source configured to provide one or more wavelengths of light. Light delivery element 155 can be constructed and arranged to deliver pulsed light, such as light delivered at a duty cycle between 0.1% and 50%. Light delivery element 155 can provide light at a power of less than 100 Watts, or less than 50 watts. In some embodiments, light delivery element 155 provides light at less than or equal to 100 Watts that is pulse-width modulated to deliver light at less than 50 Watts rms, or less than 20 Watts rms. In some embodiments, light delivery element 155 provides light at a power of at least 0.1 watts, such as at a power between 2 watts and 10 Watts. In some embodiments, light source 150 is configured to deliver light at a power of between 1.0 mW and 100 mW. In some embodiments, the light delivered by one or more light delivery elements 155 is delivered at a power between 1.0 mW and 100 mW. In some embodiments, light delivery element 155 delivers light to tissue at a power density between 1.0 mW/cm² and 10.0 mW/cm².

Light delivery element 155 can be constructed and arranged to provide one or more wavelengths of light between 300 nanometers and 900 nanometers, such as between 400 nanometers and 750 nanometers, or between 400 and 430 nanometers. Alternatively or additionally, light delivery element 155 can be constructed and arranged to provide visible light and/or ultraviolet light. Light delivery element 155 can be constructed and arranged to deliver light at varying wavelengths, such as light provided at continuously varying wavelengths or light alternative between at least a first wavelength and a second wavelength. Light delivery element 155 can be constructed and arranged to provide light at multiple wavelengths simultaneously. In some embodiments, multiple wavelengths are provided by light delivery element 155 to enhance the bactericidal effects of device 100, such as to effect multiple forms of bacteria, such as to reduce or eliminate one or more of: Escherichia coli; Klebsiella; Pseudomonas and other gram negative intestinal bacteria; Staphylococcus aureus; Streptococcus; skin bacteria; Pneumococcus; Hämophilus; respiratory tract bacteria; or anaerobic bacteria. In some embodiments, multiple wavelengths are produced or otherwise provided by light delivery element 155 to reduce or prevent an infection resulting from one or more of: a virus; a fungus; or a parasite (e.g. malaria). In some embodiments, one or more wavelengths delivered by light delivery element 155 are based on the absorption spectra of one or more microorganisms to be treated. For example, a wavelength of approximately 405 nm can be delivered to treat E. Coli; a wavelength of 470 nm can be delivered to treat staphylococcus; and/or a wavelength of 670 nm can be delivered to treat oral candida.

Light delivery element 155 can be constructed and arranged to provide light to an area (e.g. an area of tissue or urine) at a power density less than 500 mW/cm2, such as at a power density less than 250 mW/cm2, less than 100 mW/cm2, or less than 10 mW/cm2. In some embodiments, light delivery element 155 is constructed and arranged to deliver light at approximately 100 mW/cm2. In some embodiments, device 100 is constructed and arranged to deliver light to tissue at a power density of between 1.0 mW/cm² and 10 mW/cm². In some embodiments, light delivery element 155 is constructed and arranged to deliver light at a level to prevent mucosal dehydration, such as pulse-width modulated light delivery configured to prevent mucosal dehydration.

Heating of the surrounding tissue and/or fluids (e.g. urine) can be monitored by one or more sensors of device 100, such as functional element 195 configured as a temperature sensor. Functional element 195 can be positioned in or proximate balloon 125 as shown, on or in shaft 110, or at another device 100 location. Functional element 195 can be operably attached to, or operably attachable to, a sensor measurement assembly (e.g. sensor measurement assembly 200 of FIG. 1), such as via one or more conduits, not shown but typically including one or more wires or optical fibers. In some embodiments, functional element 195 comprises a thermocouple or thermistor attached to a sensor measurement assembly with one or more wires such that the sensor measurement assembly can produce a temperature measurement used to regulate the light provided by light delivery element 155. In some embodiments, functional element 195 comprises one or more optical fibers which collect infrared light (e.g. from their distal end). In these embodiments, a sensor measurement assembly can comprise an infrared sensor configured to correlate the received light to a temperature. The temperature determined by the sensor measurement assembly (e.g. via thermocouple signal, thermisters signal, infrared signal or otherwise), can be used to regulate the light delivered by light delivery element 155 in a closed loop fashion, such as to prevent or reduce mucosal dehydration and/or prevent other tissue damage as described hereabove in reference to FIG. 1.

The light delivery elements 155 of the present inventive concepts can be constructed and arranged to deliver infection reducing and/or preventing (e.g. bactericidal) light to tissue and/or body fluids, such as tissue and/or body fluids selected from the group consisting of: blood; bladder wall tissue; urethral wall tissue; urine; esophageal tissue; airway tissue; subcutaneous tissue; vascular wall tissue; cardiac valve tissue; cerebrospinal fluid; meningeal tissue; synovial fluid; and combinations of these. The light delivery elements 155 can direct light toward a skin incision, such as a skin incision through which shaft 110 passes. The light delivery elements 155 can directly light toward a body fluid such as urine. Device 100 can be constructed and arranged such that the light delivered by one or more light delivery elements 155 prevent, eliminate and/or reduces colonization of foreign material by bacteria, such as to prevent, eliminate and/or reduce a biofilm of bacteria. Alternatively or additionally, the light delivered by one or more light delivery elements can be configured to treat at least one of: a virus; a fungus or a parasite.

Light delivery element 155 can be constructed and arranged to deliver light radially out from all or a portion of balloon 125 and/or outer surface 113 of shaft 110, such as a majority portion and/or distal portion of the outer surface 113 and/or a majority portion of balloon 125. Alternatively or additionally, light delivery element 155 can be constructed and arranged to deliver light radially in from all or a portion of inner surface 114 of shaft 110, such as a majority portion and/or distal portion of the inner surface 114. In some embodiments, a light delivery element 155 is positioned to direct light toward a skin surface, such as is described hereabove in reference to light delivery element 155d of FIG. 2.

In some embodiments, bundle 154 comprises one or more optical fibers that terminate within the wall of shaft 110, such as when the material of shaft 110 has a similar refraction index as an optical fiber of bundle 154. In some embodiments, shaft 110 comprises light scattering material (e.g. titanium dioxide particles) as described herein. In some embodiments, balloon 125 is filled with fluid comprising light scattering material 158 (e.g. titanium dioxide particles) in water or other fluid, such that light delivered by bundle 154 and/or light delivery element 155 can be reflected in all directions relatively equally. In some embodiments, one or more portions of device 100, (e.g. one or more portions of the outer surface of shaft 110) comprise a light dispersing coating (e.g. a titanium dioxide coating) which may be included with a photocatalyst. For example, balloon 125 can comprise coating 128, such as a light dispersing (e.g. light scattering) coating such as titanium dioxide. Coating 128 can be positioned on the outer and/or inner surfaces of balloon 125. Titanium dioxide and/or another light dispersing component can be constructed and arranged to enhance hydrolysis, such as to generate oxygen radicals that enhance the bactericidal effects of the light delivered by device 100. In some embodiments, device 100 includes vanadium pentoxide as a photocatalyst.

Light delivery element 155, shaft 110 and/or another component of device 100 can comprise a fluorescent material, such as a material constructed and arranged to increase light dispersion. In some embodiments, the fluorescent material can be positioned at least on the outer surface 113 and/or inner surface 114 of shaft 110, such as a fluorescent coating placed upon surfaces 113 and/or 114 of shaft 110. Other coatings can be included on one or more components of device 100, such as a photosensitizer constructed and arranged to be activated by light (e.g. light delivered by a light delivery element 155) to cause a bactericidal reaction. Shaft 110 and/or balloon 125 can include one or more portions that are transparent or at least translucent (hereinafter “translucent”) to one or more wavelengths of light transmitted by a light delivery element 155. Shaft 110 can include a translucent portion surrounding a light delivery element 155 comprising an unclad portion of an optical fiber of bundle 154.

Balloon 125 can be constructed and arranged to be radially expanded through filling with fluid, e.g. fluid including light scattering material 158. Balloon 125 can be fluidly attached to inflation lumen 126 which travels proximally within shaft 110 and through housing 120 to terminate at inflation port 127. Inflation port 127 can comprise a luer or other attachment element configured to fluidly attach to a fluid delivery device such as a syringe or fluid pump, such that balloon 125 can be expanded and/or contracted such as by a user such as a clinician, nurse, patient family member or the patient. In some embodiments, inflation port 127 is attached and/or attachable to fluid delivery assembly FDA 400 as shown. Inflation port 127 can comprise one or more valves 129, such as a valve used to maintain one or more fluids in balloon 125 when FDA 400 is detached from port 127. Alternatively or additionally, one or more valves 129 can be included within one or more inflation lumens 126 and/or within balloon 125, such as to maintain fluid within balloon 125 and/or controllably release fluid from balloon 125. In some embodiments, fluid delivered into balloon 125 (e.g. via FDA 400) is used to cool one or more light delivery elements 155 positioned on or within balloon 125 or shaft 110, such as to prevent damage to tissue due to overheating. The cooling fluid can be a recirculating fluid, such as a fluid recirculated into balloon 125 in a constant fashion via two lumens (e.g. a first lumen configured as a fluid delivery lumen and a second lumen configured as a fluid extraction lumen), or via a single lumen where a reciprocating delivery and extraction of fluid is performed.

Housing 120 can be configured as a handle for a user, such as a clinician or patient, to hold while using device 100. Housing 120 can be implantable or include an implantable portion. Housing 120 can comprise a first housing and a second housing separated from the first housing. Housing 120 can surround one or more components including but not limited to: power supplies such as batteries; agent reservoirs such as pharmaceutical agent reservoirs; pumping mechanisms; energy delivery circuitry such as cardiac pacing or defibrillating circuitry; electronic processing circuitry; electronic memory circuitry; and combinations of these.

In some embodiments, device 100 comprises a device selected from the group consisting of: urine removal catheter; vascular access device; central venous catheter; peripherally inserted central catheter such as a peripherally inserted venous catheter; cerebrospinal fluid catheter; ventriculoperitoneal shunt; insulin pump; implanted device; implanted drug or other agent delivery pump; pacemaker; drive shaft assembly for a cardiac assist device; inflow and/or outflow cannula for a cardiac assist devices; neurostimulator; artificial heart; drainage catheter; colostomy tube; and combinations of these.

In some embodiments, shaft 110 is constructed and arranged for insertion through an incision of the skin, such as when shaft 110 is further inserted into a blood vessel, organ, and/or a subcutaneous tissue tunnel. In some embodiments, distal end 112 of shaft 110 is inserted into a blood vessel such that blood can be removed via lumen 115 and/or one or more agents can be delivered to an internal body location (e.g. into the cardiovascular system of the patient) via lumen 115. In these embodiments, housing 120 can include a luer or other attachment element configured to add or remove fluids via lumen 115, such as when device 100 comprises a central venous catheter or peripherally inserted central catheter. In some embodiments, shaft 110 is inserted through an incision in the skin and into the bladder, such as when device 100 is configured as a suprapubic bladder catheter. In some embodiments, shaft 110 is inserted through the skin and into an organ such as the kidney or into the ureter, such as when device 100 is configured as a urethral and/or nephrostomy catheter. In some embodiments, device 100 is configured as a colostomy tube inserted through the abdomen, such as to provide access to the intestine or colon. Alternatively, device 100 can comprise an external (i.e. non-implanted) drug delivery pump, such as a skin-attached or other external drug delivery device in which shaft 110 comprises a transcutaneous conduit (e.g. a rigid needle or flexible catheter passing through the skin surface into the subcutaneous tissue) for agent delivery. In these embodiments, housing 120 can include a pumping mechanism, not shown but described hereabove in reference to FIGS. 4 and 4A. In some embodiments, the agent delivered comprises an agent selected from the group consisting of: insulin; a chemotherapeutic agent; a nutritional material; a pain control agent such as morphine; and combinations thereof.

In some embodiments, shaft 110 is constructed and arranged for insertion into a natural body orifice, such as urethra; mouth; anus; vagina; nostril; ear hole; eye socket; and combinations of these. Device 100 can be constructed and arranged to prevent urinary tract infections. Shaft 110 can be constructed and arranged for insertion into the urethra and/or for insertion in the bladder, such as to support evacuation of urine from the patient. In some embodiments, one or more light delivery elements 155 are constructed and arranged to deliver light to a bladder, such as to a majority of the cavity of the bladder, such as to deliver light to the majority of urine in the bladder. Alternatively or additionally, one or more light delivery elements 155 can be constructed and arranged to deliver light to all or a portion of the urethra, such as to deliver light to all or a portion of the urine present in the urethra during light delivery. In some embodiments, light delivery element 155 is constructed and arranged to deliver light to a majority of the cavity of the bladder, and one or more light delivery elements 155 is constructed and arranged to deliver light to at least a distal portion of the urethra. In some embodiments, the total light delivered to the bladder is more than the total light delivered to the urethra. In some embodiments, the light delivered per area of the bladder and the urethra is approximately equal, such as at an amount less than or equal to 500 mW/cm², or less than or equal to 250 mW/cm², or less than or equal to 100 mW/cm². In some embodiments, device 100 is constructed and arranged to deliver light to tissue (e.g. the bladder and/or urethra) at a power density of between 1.0 mW/cm² and 10 mW/cm². The light provided by light delivery element 155 can be constructed and arranged to have a bactericidal effect, such as to reduce or prevent bacterial colonization, such as to reduce and/or prevent infection. Alternatively or additionally, the light provided light delivery element 155 can be constructed and arranged to treat one or more of: a virus; a fungus or a parasite.

In some embodiments, shaft 110 and housing 120 are constructed and arranged for implantation into the patient, such that light delivery element 155 can deliver light to an internal location within the patient to prevent and/or reduce infection at one or more locations surrounding shaft 110. Shaft 110 can be constructed and arranged for insertion through a subcutaneous tissue tunnel or other internal body location. Distal end 112 can be constructed and arranged for insertion into a blood vessel, a ventricle of the brain, a portion of the cerebrospinal fluid space, a joint capsule, a chamber of the heart, or other location, such as to provide a fluid conduit and/or to provide an electrical wire or optical fiber for a therapeutic application. Device 100 can comprise an implanted portion configured to deliver energy to one or more internal locations of a patient, such as to deliver energy to an organ such as a heart or brain. In these energy delivery embodiments, shaft 110 can include one or more conductors (e.g. within lumen 115) attached to an energy delivery unit within housing 120, such as the conductors and energy delivery unit described hereabove in reference to FIGS. 5 and 5A. Alternatively or additionally, device 100 can comprise an implanted portion configured to deliver a pharmaceutical agent or other agent systemically and/or locally to an internal location of the patient, such as to deliver an agent systemically or locally within the patient. The delivered agent can be configured to improve the phototherapeutic effect of light delivered by one or more light delivery elements 155. In these embodiments, an implanted housing 120 can surround a drug reservoir and pumping means which deliver an agent to lumen 115 of shaft 110, drug reservoir and pumping means not shown but described in detail hereabove in reference to FIGS. 5 and 5A.

In some embodiments, device 100 further comprises a light enhancing material 160, such as a photosensitizer and/or a photocatalyst. The light enhancing material can be included in one or more components of device 100, such as a light enhancing coating applied to all or a portion of shaft 110 and/or balloon 125. The coating can be configured to dissolve or otherwise migrate from shaft 110 and/or balloon 125. The light enhancing material can be delivered by one or more components of device 100, such as through balloon 125 when balloon 125 comprise a porous balloon, from one or more openings in shaft 110 (e.g. lumen 115 at distal end 112) or from an outlet in another component of shaft 110. In some embodiments, the light enhancing material comprises a material selected from the group consisting of: toluidine blue O; methylene blue; and combinations thereof. In some embodiments, the light enhancing material is constructed and arranged to be activated by light delivered by device 100, such as light delivered by one or more light delivery elements 155.

One or more components of device 100 can provided sterile, such as a sterile shaft 110. One or more components of device 100 can be reusable and/or re-sterilizable. In some embodiments, shaft 110 is used in a single use on a single patient, while light delivery element 155 and/or bundle 154 are reused in multiple uses with one or more patients (e.g. when bundle 154 and/or light delivery element 155 is insertable into shaft 110).

In some embodiments, in addition to one or more light delivery elements 155, device 100 includes one or more additional components constructed and arranged to further prevent and/or further reduce infection, such as functional element 195 shown in FIG. 12. Functional element 195 can be constructed and arranged to improve upon the infection prevention and/or reduction than that which is achieved with the light delivered by the one or more light delivery elements 155 alone. Functional element 195 is connected to a conduit, not shown but configured similar to conduit 196 described hereabove in reference to FIG. 1.

In some embodiments, functional element 195 comprises an element configured to deliver an electromagnetic field. Functional element 195 can produce one or more of: a dynamic electromagnetic field; a static electromagnetic field; a dynamic magnetic field; or a static magnetic field; a dynamic electrical field; or a static electrical field. Functional element 195 can produce a magnetic field with a field strength between 1 milliTesla (mT) and 500 mT. Functional element 195 can produce a magnetic field constructed and arranged to prevent adversely effecting one or more muscles and/or nerves. Alternatively or additionally, functional element 195 can comprise an element configured to deliver an electric current and/or electric potential. The delivered electric current and/or electric potential can be configured to cause electrolysis and/or otherwise modify the pH of blood or other body fluid. Alternatively or additionally, functional element 195 can comprise an ultrasound transducer, and the produced ultrasound waves can be configured to prevent (e.g. further prevent) or reduce (e.g. further reduce) an infection. Functional element 195 can be attached to an energy delivery unit, such as energy delivery unit 197 described hereabove in reference to FIG. 1. Delivery of ultrasound by functional element 195 can be configured to dissolve a biofilm, such as with a mechanical disruption action. Transmission of ultrasound waves across balloon 125 (and liquid including light scattering material 158) can improve dispersion of the ultrasound waves, similar to ultrasonic waves used to clean jewelry.

Functional element 195 can be positioned on and/or in shaft 110 and/or on or within balloon 125 as shown in FIG. 12. In some embodiments, functional element 195 comprises one or more elements positioned on and/or within balloon 125, such that functional element 195 traverses radially from shaft 110 as balloon 125 is expanded, such as when balloon 125 is expanded within a bladder to anchor shaft 110 in the bladder. In some embodiments, functional element 195 is positioned along a majority of the length of shaft 110, or along a majority of the length of a portion of shaft 110 positioned under the patient's skin. In some embodiments, functional element 195 delivers an anti-infection effect (e.g. via delivery of an electric current and/or electric potential, electromagnetic field and/or ultrasound waves) to similar locations of tissue and/or body fluid to that receiving light from the one or more light delivery elements 155. In some embodiments, functional element 195 comprises multiple functional elements, such as multiple functional elements selected from the group consisting of: one or more electromagnetic field generating elements; one or more electric current and/or electric potential delivering elements; one or more ultrasound transducers; one or more electrodes; one or more heating elements such as one or more heating elements configured to raise the temperature of blood; one or more cooling elements such as one or more cooling elements configured to lower the temperature of blood; and combinations thereof. The multiple functional elements 195 can be, singly or in combination, constructed and arranged to at least one of further prevent or further reduce infection.

One or more functional elements 195 can comprise one or more permanent magnets. In some embodiments, functional element 195 comprises multiple permanent magnets, such as multiple permanent magnets positioned on or in shaft 110 and/or on or in balloon 125. The multiple permanent magnets can be dispersed relatively uniformly along one or more portions of the length of shaft 110, such as along a full or partial circumferential portion of shaft 110, along a majority of the length of shaft 110 and/or along a majority of the length of shaft 110 which is inserted into the patient. In some embodiments, functional element 195 comprises multiple permanent magnets that are dispersed non-uniformly, such as when a higher density of magnets are positioned within and/or proximate to balloon 125 than along a more proximal portion of shaft 110.

In some embodiments, one or more functional elements 195 of device 100 are constructed and arranged to at least one of prevent or reduce infection, with or without the inclusion of one or more light delivery elements 155. In these embodiments, one or more light delivery elements 155 can be included to at least one of further prevent or further reduce infection.

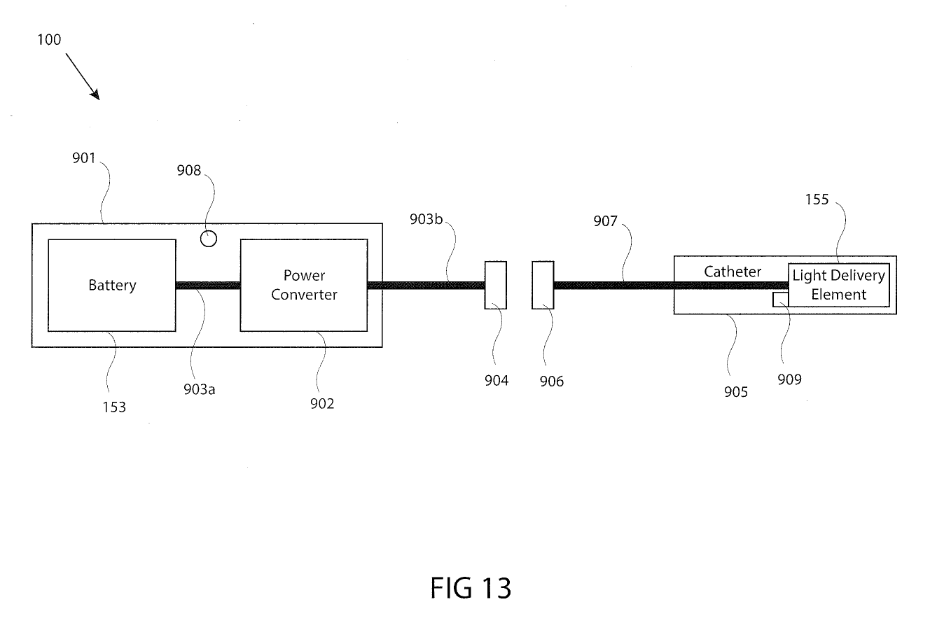

Referring now to FIG. 13, a schematic view of a device comprising an external portion including a power converter and a first connector and a catheter portion including a light delivery element and a mating, second connector is illustrated, consistent with the present inventive concepts. Device 100 comprises external portion 901 and catheter 905. External portion 901 can comprise a housing surrounding battery 153 which is electrically connected to power converter 902 via wire 903a. Battery 153 can be of similar construction and arrangement to power supply 153 described hereabove in reference to FIG. 1. Power converter 902 is electrically connected to connector 904 via wire 903b. Power converter 902 can be configured to convert an input voltage to a different output voltage, to limit current and/or to provide another power delivery function. In some embodiments, power converter 902 is configured to provide a constant current and/or a constant voltage to light delivery element 155 and/or another component of catheter 905 and/or device 100. In some embodiments, power converter 902 is configured to limit the voltage supplied to light delivery element 155 and/or to limit the current supplied to light delivery element 155. In some embodiments, power converter 902 is configured to provide constant voltage and/or current to light delivery element 155 while the voltage and/or current provided to power converter 902 by battery 153 varies, such that the energy level of light provided by light delivery element 155 is relatively constant over time. Power converter 902 can be configured to produce an output voltage between 1.5V and 20V, such as an output voltage of approximately 3.3V. Light energy delivered by light delivery element 155 can represent a portion of the power supplied by power converter 902, such as when the light energy delivered equates to between 1% and 5% of the energy supplied by power converter 902, such as when the remainder of the energy is converted to heat. In these embodiments, light delivery element 155 can be cooled by a cooling element 909. Cooling element 909 can comprise fluid surrounding light delivery element 155 (e.g. fluid contained within a balloon such as has been described herein), a silicone member or other flexible material surround light delivery element 155 and/or a metal or other heat sink (e.g. a wire) from which heat can be dissipated away from light delivery element 155. In some embodiments, light delivery element 155 delivers between 1 mW and 200 mW of light energy. Battery 153 can comprise a voltage between 1.5V and 30V, and can include multiple batteries connected in series and/or in parallel (e.g. four approximately 1.2V batteries connected in series). In some embodiments, battery 153 comprises one or more 9V batteries. Battery 153 can comprise one or more batteries, such as one or more batteries configured to provide approximately between 0.5 Ah and 20 Ah of energy. Battery 153 can comprise one or more rechargeable batteries. Power converter 902 can be configured to provide a maximum amount of current, such as a maximum current of 1000 mA.

Catheter 905 comprise a light delivery element 155, such as a light delivery element 155 positioned within an expandable balloon and configured to prevent and/or reduce infection, such as is described herein. Light delivery element 155 can be of similar construction and arrangement to light delivery element 155 of FIGS. 1, 2, 3, 4, 5A. 6 and/or 12 and/or light delivery element 555 of FIGS. 7, 10 and/or 11. Light delivery element 155 electrically attaches to connector 905 via wire 907.

Connector 904 can comprise a male and/or female connector (e.g. including one or more male or female connecting portions) configured to electrically attach to a mating connector 906 of catheter 905, such that energy provided by battery 153 and converted by power converter 902 can be delivered to light delivery element 155, through connections made between connector 904 and connector 906.

In some embodiments, device 100 includes an element configured to alert an operator (e.g. a clinician and/or the patient) that light delivery element 155 is delivering light, such as indicator 908 shown. Indicator 908 can comprise a visible light LED or other visible indicator that changes status (e.g. turns on) when light delivery element 155 begins delivering light.

The devices of the present inventive concepts include one or more light or other energy delivery elements constructed and arranged to deliver light or other energy. Energy delivery can be configured to provide phototherapy and/or thermal therapy. Energy delivery can be configured to reduce or prevent infection, such as by delivering light with a bactericidal effect, or by delivering light to treat one or more of: a virus; a fungus; or a parasite (e.g. malaria). The energy delivery elements can include a source of light (e.g. an LED) and/or they can be attached to a source of light (e.g. attached to a laser, LED or other light source via an optical fiber). Light can be delivered from the light source in a continuous or pulsed (e.g. pulse-width modulated) manner. The light source can provide light configured to have bactericidal effects on one or more forms of bacteria, such as bacteria selected from the group consisting of: Escherichia coli; Klebsiella; Pseudomonas and other gram negative intestinal bacteria; Staphylococcus aureus; Streptococcus; a skin bacteria; Pneumococcus; Hämophilus; a respiratory tract bacteria; anaerobic bacteria; and combinations of these. The light source can produce or otherwise provide light configured to reduce or prevent an infection resulting from one or more of: a virus; a fungus; or a parasite (e.g. malaria). Delivery of light from the light source can be performed in a closed-loop fashion, such as when a device of the present inventive concepts includes at least one sensor such as a temperature sensor, and light is delivered based on one or more measurements from one or more sensors.

Light can be delivered from a light delivery element in a symmetric or asymmetric pattern. The light can be delivered to tissue and/or body fluids, such as tissue and/or body fluids selected from the group consisting of: blood; bladder wall tissue; urethral wall tissue; urine; esophageal tissue; airway tissue; subcutaneous tissue; vascular wall tissue; cardiac valve tissue; cerebrospinal fluid; meningeal tissue; synovial fluid; and combinations of these. Light can be delivered to one or more light delivery elements via an optical fiber, such as via multiple optical fibers that each travel distally from the proximal end of the device to a different light delivery element, or a single optical fiber that travels distally and proximally (i.e. back and forth) to deliver light to two or more light delivery elements. The light delivery elements can comprise one or more optical components, such as an optical component selected from the group consisting of: optical fiber; lens; ball lens; prism; diffractor; filter; mirror; and combinations of these. In some embodiments, the light delivery element comprises a ball lens positioned at the end of an optical fiber. In some embodiments, one or more light delivery elements include light scattering material configured to scatter light produced by or delivered to the light delivery element. In some embodiments, one or more light delivery elements are rotated, such as by rotating an optical fiber. The devices of the present inventive concepts can include one or more shafts configured for insertion into the body of a patient, such as via being implanted within the patient, being inserted through an incision of the patient or being inserted into a natural orifice such as the urethra. The shafts can be flexible, rigid, or include both flexible and rigid portions. The shafts can include one or more lumens, such as one or more lumens comprising an opening constructed and arranged to withdraw blood or other body fluids and/or deliver one or more agents to an internal body location. Alternatively, the lumens can be filled with material, such as one or more conduits comprising one or more wires, optical fibers and/or other conduits. The devices of the present inventive concepts can include one or more additional components constructed and arranged to further prevent and/or further reduce infection, such as a functional element configured to deliver an electric current and/or electric potential, an electromagnetic field and/or ultrasound waves. The devices of the present inventive concepts can include one or more single use components (e.g. a single use shaft) and/or one or more reusable components (e.g. a single use light source).

The light and/or energy delivery devices of the present inventive concepts can be constructed for short-term clinical use with the patient (e.g. use for less than 16 hours, less than 24 hours, less than 3 days or less than 7 days), or for long-term clinical use (e.g. use for at least 1 week, at least 1 month, at least 3 months or at least 6 months). The light and/or other energy delivery elements of the present inventive concepts can be constructed and arranged to deliver light (e.g. in a continuous or pulsed, intermittent manner) to reduce and/or prevent infection for short durations of time (e.g. less than 30 minutes, less than 1 hour, less than 4 hours, less than 16 hours, less than 24 hours, less than 3 days or less than 7 days), or for long-term use (e.g. at least 1 week, at least 1 month or at least 3 months). In some embodiments, one or more energy delivery elements are constructed and arranged to deliver light for or at least 6 months, such as when the one or more energy delivery elements are implanted in the patient. The energy delivery elements of the present inventive concepts can be constructed and arranged to be implanted within the patient, remain outside the patient's skin, or pass through the patient's skin via an incision or natural body orifice.

While the preferred embodiments of the devices and methods have been described in reference to the environment in which they were developed, they are merely illustrative of the principles of the inventions. Modification or combinations of the above-described assemblies, other embodiments, configurations, and methods for carrying out the invention, and variations of aspects of the invention that are obvious to those of skill in the art are intended to be within the scope of the claims. In addition, where this application has listed the steps of a method or procedure in a specific order, it may be possible, or even expedient in certain circumstances, to change the order in which some steps are performed, and it is intended that the particular steps of the method or procedure claim set forth herebelow not be construed as being order-specific unless such order specificity is expressly stated in the claim.

1. A device for insertion into a mammalian patient comprising:
a shaft comprising a proximal end, a distal end and a lumen therebetween, wherein the shaft further comprises an outer surface and an inner surface; and
a light delivery element constructed and arranged to deliver light to perform a function selected from the group consisting of:
prevent infection;
reduce infection;
cause a physiologic effect selected from the group consisting of:
blood temperature increase; vasodilation;
an increase in local nitric oxide;
enhance nitric oxide release from the vascular endothelium;
prolong local nitric oxide effects; an alteration in the function of erythrocytes;
a modification in oxygen release from hemoglobin;
a modification in pH of blood;
a modulation in the immune response of blood leucocytes;
a modulation of the coagulation and/or thrombocyte function;
a modification in the function of heme catalyst enzymes in the blood;
a modification in hormonal action of a peptide and/or non-peptide hormone;
a modification in the binding capacity of one or more antibodies;
a decrease in blood glucose level;
affect circulating tumor cells by selective heating, photocoagulation and/or photolysis; and combinations thereof; and
combinations thereof.

2-348. (canceled)
